SEQUENCE LISTING (19 sequences)

SEQ ID NO: 1 - DNA, 837 bp, Zea mays
  misc_feature (0)...(0): LEC1
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtgatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 2 - protein, 278 aa, Zea mays
MDSSSFLPAAGAENGSAAGGANNGGAAQQHAAPAIREQDRLMPIANVIRIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 3 - DNA, 1927 bp, Zea mays
  misc_feature (0)...(0): AGP2
aagcaaaacgccaaacaggctcctctgaaactcccagagccggtagaggcacgccactccgcagtgtcagtcactgcccccccatcacgccgcccgccctttctcggccgttgccgcgagctcgcttcgacccaccgccgatggcgacggcgatgggcgcgatggccgccacctgcatggcctcgatcccggccgccgctacgttccccggtgatctcggcgtcggacgacgccgagcggttgtaccagggtggcgcgccggtgggagacggctgcgtgcgacgccgcctgcccggaggccgttcctgttctcgccgaagggcgtttcggactcgcggagctcgcaaacgtgccttgatccggacgccagcacgagtgttcttgggatcatccttggagggggtgctgggacaaggctgtatccactgacgaagaagagggcgaaaccagcagtgccgttgggcgccaactacaggctcatagatatccctgtcagcaactgtctcaacagtaacgtctccaagatatatgtgctaacacagttcaactctgcttcgctcaaccgccacctctcgagggcctatggaaacaacattgccgggtacaagaatgagggattcgttgaggtccttgcagcacaacagagtccagagaatcccaactggtttcagggtactgcagatgccgtgcgtcaatatatgtggctatttgaggagcataatatcatggagttccttattctggctggagatcatctgtaccgtatggattaccaaaagttcattcaagctcatagagaaacagatgctgatataacagttgcagccctgccaatggatgaacaacgtgcaactgcatttggtcttatgaaaattgatgatgaagggaggatagttgagtttgcagaaaaaccaaaaggagagaagctgagatcaatgatggtagacaccactatattgggccttgatcctgaaagggccaaagaactgccttatattgccagtatgggaatctatgtttttagcaaagatgtgatgcttcgtcttctcagagaaaattttcctgcagcaaatgactttggaagtgaggttattcctggcgcaacagaaattggattaagggtgcaagcttacttatatgatggttactgggaagatattggtactattgaagcattttataatgcaaacttgggaataaccaagaagcctgtaccggattttagcttctatgatcgttctgctccaatttatacgcaacctagatatttgcctccttcgaaggttcttgatgctgatgtgacagacagtgttattggagaagggtgtgttattaaacattgcacaatcaatcattccgtagttggactccgttcctgcatttctgaaggtgcagttatagaggactctttgctaatgggtgcagactattacgagactgagaatgataagaatgtcctttctgagactggtggcattcccattggtattgggaaaaattcacatatcagaaaagcaataattgacaaaaatgctcgcattggagaaaatgtgaagataatcaattttgataatgtccaagaagcagtaagggagacagaaggatattttatcaaaagtggaattgtcacggtgattaaagatgctttaattcctagtggaacaatcatataaagtcagccaacaccatgtatataaggttgggttgcatcatatggtgatcgccaatgcgcatcaagttctctcatcccttgcgagaccatgcttatttccattgcaaaatcatcaagctctgcatcaacttgttgtggataataatgaaataataaatggtgccattccatggcaacatttttctgtcactagtatgaggcactgttctaaatgtactgttgagtggaatttgaatatgtatgcttttgcaatcg

SEQ ID NO: 4 - protein, 510 aa, Zea mays
MATAMGAMAATCMASIPAAATFPGDLGVGRRRAVVPGWRAGGRRLRATPPARRPFLFSPKGVSDSRSSQTCLDPDASTSVLGIILGGGAGTRLYPLTKKRAKPAVPLGANYRLIDIPVSNCLNSNVSKIYVLTQFNSASLNRHLSRAYGNNIAGYKNEGFVEVLAAQQSPENPNWFQGTADAVRQYMWLFEEHNIMEFLILAGDHLYRMDYQKFIQAHRETDADITVAALPMDEQRATAFGLMKIDDEGRIVEFAEKPKGEKLRSMMVDTTILGLDPERAKELPYIASMGIYVFSKDVMLRLLRENFPAANDFGSEVIPGATEIGLRVQAYLYDGYWEDIGTIEAFYNANLGITKKPVPDFSFYDRSAPIYTQPRYLPPSKVLDADVTDSVIGEGCVIKHCTINHSVVGLRSCISEGAVIEDSLLMGADYYETENDKNVLSETGGIPIGIGKNSHIRKAIIDKNARIGENVKIINFDNVQEAVRETEGYFIKSGIVTVIKDALIPSGTII

SEQ ID NO: 5 - DNA, 1412 bp, Artificial Sequence
  Truncated Zea mays AGP2
aagcaaaacgccaaacaggctcctctgaaactcccagagccggtagaggcacgccactccgcagtgtcagtcactgcccccccatcacgccgcccgccctttctcggccgttgccgcgagctcgcttcgacccaccgccgatggcgacggcgatgggcgcgatggccgccacctgcatggcctcgatcccggccgccgctacgttccccggtgatctcggcgtcggacgacgccgagcggttgtaccagggtggcgcgccggtgggagacggctgcgtgcgacgccgcctgcccggaggccgttcctgttctcgccgaagggcgtttcggactcgcggagctcgcaaacgtgccttgatccggacgccagcacgagtgttcttgggatcatccttggagggggtgctgggacaaggctgtatccactgacgaagaagagggcgaaaccagcagtgccgttgggcgccaactacaggctcatagatatccctgtcagcaactgtctcaacagtaacgtctccaagatatatgtgctaacacagttcaactctgcttcgctcaaccgccacctctcgagggcctatggaaacaacattgccgggtacaagaatgagggattcgttgaggtccttgcagcacaacagagtccagagaatcccaactggtttcagggtactgcagatgccgtgcgtcaatatatgtggctatttgaggagcataatatcatggagttccttattctggctggagatcatctgtaccgtatggattaccaaaagttcattcaagctcatagagaaacagatgctgatataacagttgcagccctgccaatggatgaacaacgtgcaactgcatttggtcttatgaaaattgatgatgaagggaggatagttgagtttgcagaaaaaccaaaaggagagaagctgagatcaatgatggtagacaccactatattgggccttgatcctgaaagggccaaagaactgccttatattgccagtatgggaatctatgtttttagcaaagatgtgatgcttcgtcttctcagagaaaattttcctgcagcaaatgactttggaagtgaggttattcctggcgcaacagaaattggattaagggtgcaagcttacttatatgatggttactgggaagatattggtactattgaagcattttataatgcaaacttgggaataaccaagaagcctgtaccggattttagcttctatgatcgttctgctccaatttatacgcaacctagatatttgcctccttcgaaggttcttgatgctgatgtgacagacagtgttattggagaagggtgtgttattaaacattgcacaatcaatcattccgtagttggactccgttcctgcatttctgaaggtgcagttatagaggactcttaa

SEQ ID NO: 6 - protein, 423 aa, Zea mays
MATAMGAMAATCMASIPAAATFPGDLGVGRRRAVVPGWRAGGRRLRATPPARRPFLFSPKGVSDSRSSQTCLDPDASTSVLGIILGGGAGTRLYPLTKKRAKPAVPLGANYRLIDIPVSNCLNSNVSKIYVLTQFNSASLNRHLSRAYGNNIAGYKNEGFVEVLAAQQSPENPNWFQGTADAVRQYMWLFEEHNIMEFLILAGDHLYRMDYQKFIQAHRETDADITVAALPMDEQRATAFGLMKIDDEGRIVEFAEKPKGEKLRSMMVDTTILGLDPERAKELPYIASMGIYVFSKDVMLRLLRENFPAANDFGSEVIPGATEIGLRVQAYLYDGYWEDIGTIEAFYNANLGITKKPVPDFSFYDRSAPIYTQPRYLPPSKVLDADVTDSVIGEGCVIKHCTINHSVVGLRSCISEGAVIEDS

SEQ ID NO: 7 - DNA, 1557 bp, Zea mays
  misc_feature (0)...(0): AGP1
atgcagttcagcagcgtgcttcccctagagggaaaagcgtgtatgagcccagtgaggagaggcagtggaggttatgggagtgagaggatgaggatcaactgctgcagcatcaggcgcaacaaggcactgaggaggatgtgtttcagtgcaaggggtgctgtgagcagcacgcagtgtgtgctcacatcagatgctggcccagacactcttgtcgtccgaacatcctttcggaggaattatgctgatcctaatgaagtcgctgccgtcattttgggtggtggtaccgggactcagcttttccctctcacaagcacaagggccacccctgctgttcctattggaggatgttacaggcttattgatatccccatgagcaactgtttcaacagtggcataaacaagatatttgttatgactcagttcaactcagcttctcttaaccgtcacattcatcgtacctatcttggtggggggatcaacttcactgatggatctgttgaggtgctggctgcaacacaaatgcctggggaggctgctggttggttccagggcacagcagacgccgttagaaaatttatctgggtacttgaggattattacaagcataaagctatagaacacattttgattttgtcaggagatcaactctatcgtatggattacatggagcttgtgcagaaacatgtcgatgacaatgcagacataactttatcatgcgctcctgttggagagagtcgagcatctgactatggattagttaagttcgatagttcaggccgtgtaattcagttctctgagaaaccaaagggtgctgccttggaagaaatgaaagtggataccagcttcctcaatttcgccattgattccccagctgaatatccctatatcgcttcaatgggagtttacgtttttaagagagatgttttgttagaccttctaaagtcacggtatgctgaactgcatgactttggttctgaaattctgcccaaggctttgcatgagcacaatgtacaggcatatgttttcactgactactgggaggacattggaacaatcagatctttctttgatgcaaacatggccctctgcgagcagcctccaaagttcgagttttacgatccgaaaacaccattcttcacttcccctcggtacttgccaccaacgaagtcggataagtgcaggattaaagacgcgatcatttcacacggctgcttcttgcgtgagtgtgccatcgagcactctattgttggtgttcgctcacgcctaaactctggatgcgagctcaagaataccatgatgatgggtgcggatttgtatgagaccgaagacgagatctcaaggctactggcagagggcaaggtgcccattggcgtaggggagaacacgaagataagcaactgcatcatcgacatgaatgccagggttggaaggaacgtctccataacaaacaaagagggcgtccaagaagccgaccggccggacgaaggatactacatccggtctgggatcgtggtagtcctgaagaacgcaaccatcaaggacggcaccgtcatatag

SEQ ID NO: 8 - protein, 518 aa, Zea mays
MQFSSVLPLEGKACMSPVRRGSGGYGSERMRINCCSIRRNKALRRMCFSARGAVSSTQCVLTSDAGPDTLVVRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFQGTADAVRKFIWVLEDYYKHKAIEHILILSGDQLYRMDYMELVQKHVDDNADITLSCAPVGESRASDYGLVKFDSSGRVIQFSEKPKGAALEEMKVDTSFLNFAIDSPAEYPYIASMGVYVFKRDVLLDLLKSRYAELHDFGSEILPKALHEHNVQAYVFTDYWEDIGTIRSFFDANMALCEQPPKFEFYDPKTPFFTSPRYLPPTKSDKCRIKDAIISHGCFLRECAIEHSIVGVRSRLNSGCELKNTMMMGADLYETEDEISRLLAEGKVPIGVGENTKISNCIIDMNARVGRNVSITNKEGVQEADRPDEGYYIRSGIVVVLKNATIKDGTVI

SEQ ID NO: 9 - DNA, 1500 bp, Artificial Sequence
  Truncated Zea mays AGP1
atgcagttcagcagcgtgcttcccctagagggaaaagcgtgtatgagcccagtgaggagaggcagtggaggttatgggagtgagaggatgaggatcaactgctgcagcatcaggcgcaacaaggcactgaggaggatgtgtttcagtgcaaggggtgctgtgagcagcacgcagtgtgtgctcacatcagatgctggcccagacactcttgtcgtccgaacatcctttcggaggaattatgctgatcctaatgaagtcgctgccgtcattttgggtggtggtaccgggactcagcttttccctctcacaagcacaagggccacccctgctgttcctattggaggatgttacaggcttattgatatccccatgagcaactgtttcaacagtggcataaacaagatatttgttatgactcagttcaactcagcttctcttaaccgtcacattcatcgtacctatcttggtggggggatcaacttcactgatggatctgttgaggtgctggctgcaacacaaatgcctggggaggctgctggttggttccagggcacagcagacgccgttagaaaatttatctgggtacttgaggattattacaagcataaagctatagaacacattttgattttgtcaggagatcaactctatcgtatggattacatggagcttgtgcagaaacatgtcgatgacaatgcagacataactttatcatgcgctcctgttggagagagtcgagcatctgactatggattagttaagttcgatagttcaggccgtgtaattcagttctctgagaaaccaaagggtgctgccttggaagaaatgaaagtggataccagcttcctcaatttcgccattgattccccagctgaatatccctatatcgcttcaatgggagtttacgtttttaagagagatgttttgttagaccttctaaagtcacggtatgctgaactgcatgactttggttctgaaattctgcccaaggctttgcatgagcacaatgtacaggcatatgttttcactgactactgggaggacattggaacaatcagatctttctttgatgcaaacatggccctctgcgagcagcctccaaagttcgagttttacgatccgaaaacaccattcttcacttcccctcggtacttgccaccaacgaagtcggataagtgcaggattaaagacgcgatcatttcacacggctgcttcttgcgtgagtgtgccatcgagcactctattgttggtgttcgctcacgcctaaactctggatgcgagctcaagaataccatgatgatgggtgcggatttgtatgagaccgaagacgagatctcaaggctactggcagagggcaaggtgcccattggcgtaggggagaacacgaagataagcaactgcatcatcgacatgaatgccagggttggaaggaacgtctccataacaaacaaagagggcgtccaagaagccgaccggccggacgaaggatactacatccgg

SEQ ID NO: 10 - protein, 500 aa, Artificial Sequence
  Truncated Zea mays AGP1
MQFSSVLPLEGKACMSPVRRGSGGYGSERMRINCCSIRRNKALRRMCFSARGAVSSTQCVLTSDAGPDTLVVRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFQGTADAVRKFIWVLEDYYKHKAIEHILILSGDQLYRMDYMELVQKHVDDNADITLSCAPVGESRASDYGLVKFDSSGRVIQFSEKPKGAALEEMKVDTSFLNFAIDSPAEYPYIASMGVYVFKRDVLLDLLKSRYAELHDFGSEILPKALHEHNVQAYVFTDYWEDIGTIRSFFDANMALCEQPPKFEFYDPKTPFFTSPRYLPPTKSDKCRIKDAIISHGCFLRECAIEHSIVGVRSRLNSGCELKNTMMMGADLYETEDEISRLLAEGKVPIGVGENTKISNCIIDMNARVGRNVSITNKEGVQEADRPDEGYYIR

SEQ ID NO: 11 - DNA, 1615 bp, Arabidopsis thaliana
  misc_feature (0)...(0): FAD2
gaccaccagaagaagagccacacactcacaaattaaaaagagagagagagagagagagacagagagagagagagattctgcggaggagcttcttcttcgtagggtgttcatcgttattaacgttatcgcccctacgtcagctccatctccagaaacatgggtgcaggtggaagaatgccggttcctacttcttccaagaaatcggaaaccgacaccacaaagcgtgtgccgtgcgagaaaccgcctttctcggtgggagatctgaagaaagcaatcccgccgcattgtttcaaacgctcaatccctcgctctttctcctaccttatcagtgacatcattatagcctcatgcttctactacgtcgccaccaattacttctctctcctccctcagcctctctcttacttggcttggccactctattgggcctgtcaaggctgtgtcctaactggtatctgggtcatagcccacgaatgcggtcaccacgcattcagcgactaccaatggctggatgacacagttggtcttatcttccattccttcctcctcgtcccttacttctcctggaagtatagtcatcgccgtcaccattccaacactggatccctcgaaagagatgaagtatttgtcccaaagcagaaatcagcaatcaagtggtacgggaaatacctcaacaaccctcttggacgcatcatgatgttaaccgtccagtttgtcctcgggtggcccttgtacttagcctttaacgtctctggcagaccgtatgacgggttcgcttgccatttcttccccaacgctcccatctacaatgaccgagaacgcctccagatatacctctctgatgcgggtattctagccgtctgttttggtctttaccgttacgctgctgcacaagggatggcctcgatgatctgcctctacggagtaccgcttctgatagtgaatgcgttcctcgtcttgatcacttacttgcagcacactcatccctcgttgcctcactacgattcatcagagtgggactggctcaggggagctttggctaccgtagacagagactacggaatcttgaacaaggtgttccacaacattacagacacacacgtggctcatcacctgttctcgacaatgccgcattataacgcaatggaagctacaaaggcgataaagccaattctgggagactattaccagttcgatggaacaccgtggtatgtagcgatgtatagggaggcaaaggagtgtatctatgtagaaccggacagggaaggtgacaagaaaggtgtgtactggtacaacaataagttatgaggatgatggtgaagaaattgtcgacctttctcttgtctgtttgtcttttgttaaagaagctatgcttcgttttaataatcttattgtccattttgttgtgttatgacattttggctgctcattatgttatgtgggaagttagtgttcaaatgttttgtgtcggtattgttcttctcatcgctgttttgttgggatcgtagaaatgtgaccttcggacagtaaaactcttgtactaaaactatctccctattggcatttcttaaacttttaatagttacgtgctcgtagtgaatcttgacttgagtca

SEQ ID NO: 12 - DNA, 1768 bp, Zea mays
  CDS (164)...(1328)
cggcctctcccctccctcctccctgcaaatcctgcagacaccaccgctcgtttttctctccgggacaggagaaaaggggagagagaggtgaggcgcggtgtccgcccgatctgctctgcccgacgcagctgttacgacctcctcagtctcagtcaggagcaagatgggtgccggcggcaggatgaccgagaaggagcgggagaagcaggagcagctcgcccgagctaccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactactcgctcctggacgacgtggtcggcctggtgctgcactcgtcgctcatggtgccctacttctcgtggaagtacagccaccggcgccaccactccaacacggggtccctggagcgcgacgaggtgttcgtgcccaagaagaaggaggcgctgccgtggtacaccccgtacgtgtacaacaacccggtcggccgggtggtgcacatcgtggtgcagctcaccctcgggtggccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctagcgctcgcagagctgaggacgctaccgtaggaatgggagcagaaaccaggaggaggagagtactcgccccaaagtctccgtcaacctatctaatcgttagtcgtcagtcttttagacgaagagagatcatttgggcacagagacgaaggcttactgcagtgccatcgctagagctcatcaagtacaagtaggcaaattcgtcaacttagtgtgtcccatgttgtttttcttaggtccgctgctgtaggctttccggcggcggtcgtttgtgtggttggcatccgtggccatctgtgcgtgcgtggccgcgcttgtcgtgtgcgtctgtcgtcgcgttggcgtcgtctctgtgctccccgtgtgttgttgtaaaacaagaagatgttttctggtgtctttggcggaatcagatcgtccgaacgaaaaaaaaaaaaaaaaaaaa

SEQ ID NO: 13 - protein, 387 aa, Zea mays
MGAGGRMTEKEREKQEQLARATGGAAMQRSPVEKPPFTLGQIKKAIPPHCFERSVLKSFSYVVHDLVIAAALLYFALAIIPALPSPLRYAAWPLYWIAQGCVCTGVWVIAHECGHHAFSDYSLLDDVVGLVLHSSLMVPYFSWKYSHRRHHSNTGSLERDEVFVPKKKEALPWYTPYVYNNPVGRVVHIVVQLTLGWPLYLATNASGRPYPRFACHFDPYGPIYNDRERAQIFVSDAGVVAVAFGLYKLAAAFGVWWVVRVYAVPLLIVNAWLVLITYLQHTHPSLPHYDSSEWDWLRGALATMDRDYGILNRVFHNITDTHVAHHLFSTMPHYHAMEATKAIRPILGDYYHFDPTPVAKATWREAGECIYVEPEDRKGVFWYNKKF

SEQ ID NO: 14 - protein, 65 aa, Artificial Sequence
  LEC1 consensus protein sequence
REQDXXMPIANVIRIMRXXLPXHAKISDDAKEXIQECVSEYISFXTXEANXRCXXXXRKTXXXEX

SEQ ID NO: 15 - DNA, 1430 bp, Artificial Sequence
  misc_feature (1)...(686): Truncated AGP2 nucleotide sequence
aattcgcccttggcaccagtgacgcgaaagcaaaacgccaaacaggctcctctgaaactcccagagccggtagaggcacgccactccgcagtgtcagtcactgcccccccatcacgccgcccgccctttctcggccgttgccgcgagctcgctccgacccaccgccgatggcgacggcgatgggcgcgatggccgccacctgcatggcctcgatcccggccgccgctacgttccccggtgatctcggcgtcggacgacgccgagcggttgtaccagggtggcgcgccggtgggagacggctgcgtgcgacgccgcctgcccggaggccgttcctgttctcgccgaagggcgtttcggactcgcggagctcgcaaacgtgccttgatccggacgccagcacgagtgttcttgggatcatccttggagggggtgctgggacaaggctgtatccactgacgaagaagagggcgaaaccagcagtgccgttgggcgccaactacaggctcatagatatccctgtcagcaactgtctcaacagtaacgtctccaagatatatgtgctaacacagttcaactctgcttcgctcaaccgccacctctcgagggcctatggaaacaacattgccgggtacaagaatgagggattcgttgaggtccttgcagcacaacagagtccagagaatcccaattgggaagaaatgaaagtggataccagcttcctcaatttcgccattgattccccagctgaatatccctatatcgcttcaatgggagtttacgtttttaagagagatgttttgttagaccttctaaagtcacggtatgctgaactgcatgactttggttctgaaattctgcccaaggctttgcatgagcacaatgtacaggcatatgttttcactgactactgggaggacattggaacaatcagatctttctttgatgcaaacatggccctctgcgagcagcctccaaagttcgagttttacgatccgaaaacaccattcttcacttcccctcggtacttgccaccaacgaagtcggataagtgcaggattaaagacgcgatcatttcacacggctgcttcttgcgtgagtgtgccatcgagcactctattgttggtgttcgctcacgcctaaactctggatgcgagctcaagaataccatgatgatgggtgcggatttgtatgagaccgaagacgagatctcaaggctactggcagagggcaaggtgcccattggcgtaggggagaacacgaagataagcaactgcatcatcgacatgaatgccagggttggaaggaacgtctccataacaaacaaagagggcgtccaagaagccgaccggccggacgaaggatactacatccggtctgggatcgtggtagtcctgaagaacgcaaccatcaaggacggcaccgtcatatag

SEQ ID NO: 16 - DNA, 1669 bp, Artificial Sequence
  misc_feature (1)...(167): AGP2 5′ untranslated region
aattcgcccttggcaccagtgacgcgaaagcaaaacgccaaacaggctcctctgaaactcccagagccggtagaggcacgccactccgcagtgtcagtcactgcccccccatcacgccgcccgccctttctcggccgttgccgcgagctcgctccgacccaccgccgatggcgacggcgatgggcgcgatggccgccacctgcatggcctcgatcccggccgccgctacgttccccggtgatctcggcgtcggacgacgccgagcggttgtaccagggtggcgcgccggtgggagacggctgcgtgcgacgccgcctgcccggaggccgttcctgttctcgccgaagggcgtttcggactcgcggagctcgcaaacgtgccttgatccggacgccagcacgagtgttcttgggatcatccttggagggggtgctgggacaaggctgtatccactgacgaagaagagggcgaaaccagcagtgccgttgggcgccaactacaggctcatagatatccctgtcagcaactgtctcaacagtaacgtctccaagatatatgtgctaacacagttcaactctgcttcgctcaaccgccacctctcgagggcctatggaaacaacattgccgggtacaagaatgagggattcgttgaggtccttgcagcacaacagagtccagagaatcccaactggtttcagggtactgcagatgccgtgcgtcaatatatgtggctatttgaggagcataatatcatggagttccttattctggctggagatcatctgtaccgtatggattaccaaaagttcattcaagctcatagagaaacagatgctgatataacagttgcagccctgccaatggatgaacaacgtgcaactgcatttggtcttatgaaaattgatgatgaagggaggatagttgagttcgcagaaaaaccaaaaggagagaagctgagatcaatgatggtagacaccactatattgggccttgatcctgaaagggccaaagaactgccttatattgctagtatgggaatctatgtttttagcaaagatgtgatgcttcggcttctcagagaaaattttcctgcagcaaatgactttggaagtgaggttattcctggcgcaacagaaattggattaagttaacaagctatctatgagcctgtagttggcgcccaacggcactgctggtttcgccctcttcttcgtcagtggatacagccttgtcccagcaccccctccaaggatgatcccaagaacactcgtgctggcgtccggatcaaggcacgtttgcgagctccgcgagtccgaaacgcccttcggcgagaacaggaacggcctccgggcaggcggcgtcgcacgcagccgtctcccaccggcgcgccaccctggtacaaccgctcggcgtcgtccgacgccgagatcaccggggaacgtagcggcggccgggatcgaggccatgcaggtggcggccatcgcgcccatcgccgtcgccatcggcggtgggtcggagcgagctcgcggcaacggccgagaaagggcgggcggcgtgatgggggggcagtgactgacactgcggagtggcgtgcctctaccggctctgggagtttcagaggagcctgtttggcgttttgctttcgcgtcactggtgccaagggcgaatt

SEQ ID NO: 17 - DNA, 1170 bp, Artificial Sequence
  misc_feature (1)...(298): Zea mays FAD2 trucated nucleotide sequence region 2
ccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactagccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctagtagtccgagaaggcgtggtggccgcactcgtgcgcgatgacccacacgccggtgcacacgcacccctgcgcgatccagtacagcggccaggcggcgtagcggagcgggcttgggagcgccggtatgatggccagcgcgaagtagaggagcgccgcggcgatcaccaggtcgtggaccacgtacgagaaggacttgagcaccgagcgctcgaagcagtgtggcgggatggccttcttgatctgacccagagtgaacggaggcttctccaccggcgaccgctgcatcgcggcgccaccgg

SEQ ID NO: 18 - DNA, 2855 bp, Artificial Sequence
  misc_feature (1)...(167): AGP2 5′ untranslated region
aattcgcccttggcaccagtgacgcgaaagcaaaacgccaaacaggctcctctgaaactcccagagccggtagaggcacgccactccgcagtgtcagtcactgcccccccatcacgccgcccgccctttctcggccgttgccgcgagctcgctccgacccaccgccgatggcgacggcgatgggcgcgatggccgccacctgcatggcctcgatcccggccgccgctacgttccccggtgatctcggcgtcggacgacgccgagcggttgtaccagggtggcgcgcgcgccggtgggagacggctgcgtgcgacgccgcctgcccggaggccgttcctgttctcgccgaagggcgtttcggactcgcggagctcgcaaacgtgccttgatccggacgccagcacgagtgttcttgggatcatccttggagggggtgctgggacaaggctgtatccactgacgaagaagagggcgaaaccagcagtgccgttgggcgccaactacaggctcatagatctagacccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactagccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccgggcagccatggtcaatcactagtgaattcgcggccgcctgcaggtcgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctagtagtccgagaaggcgtggtggccgcactcgtgcgcgatgacccacacgccggtgcacacgcacccctgcgcgatccagtacagcggccaggcggcgtagcggagcgggcttgggagcgccggtatgatggccagcgcgaagtagaggagcgccgcggcgatcaccaggtcgtggaccacgtacgagaaggacttgagcaccgagcgctcgaagcagtgtggcgggatggccttcttgatctgacccagagtgaacggaggcttctccaccggcgaccgctgcatcgcggcgccaccgggtaataaaagggtagtgtacgcctaccgcgtacaacaagctatctatgagcctgtagttggcgcccaacggcactgctggtttcgccctcttcttcgtcagtggatacagccttgtcccagcaccccctccaaggatgatcccaagaacactcgtgctggcgtccggatcaaggcacgtttgcgagctccgcgagtccgaaacgcccttcggcgagaacaggaacggcctccgggcaggcggcgtcgcacgcagccgtctcccaccggcgcgcgcgccaccctggtacaaccgctcggcgtcgtccgacgccgagatcaccggggaacgtagcggcggccgggatcgaggccatgcaggtggcggccatcgcgcccatcgccgtcgccatcggcggtgggtcggagcgagctcgcggcaacggccgagaaagggcgggcggcgtgatgggggggcagtgactgacactgcggagtggcgtgcctctaccggctctgggagtttcagaggagcctgtttggcgttttgctttcgcgtcactggtgccaagggcgaatt

SEQ ID NO: 19 - DNA, 1262 bp, Artificial Sequence
  Plasmid F  FAD2 inverted repeat (IR)
atgaccgagaaggagcgggagaagcaggagcagctcgcccgagctaccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactagccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctagtagtccgagaaggcgtggtggccgcactcgtgcgcgatgacccacacgccggtgcacacgcacccctgcgcgatccagtacagcggccaggcggcgtagcggagcgggcttgggagcgccggtatgatggccagcgcgaagtagaggagcgccgcggcgatcaccaggtcgtggaccacgtacgagaaggacttgagcaccgagcgctcgaagcagtgtggcgggatggccttcttgatctgacccagagtgaacggaggcttctccaccggcgaccgctgcatcgcggcgccaccgggcggtccgattaaactttaattcggaccgaagcttgcatgcctgca

(19) United States
(12) Patent Application Publication
(10) Pub. No.: US 2005/0160494 A1
(43) Pub. Date: Jul. 21, 2005

(54) Alteration of oil traits in plants

(75) Inventors: George W. Singletary, Ankeny, IA (US); Peter D. Coaldrake, Johnston, IA (US); Paulette M. Krumpelman, Urbandale, IA (US); Douglas S. Nubel, Clive, IA (US); Court Saunders, Clive, IA (US); Mitchell C. Tarczynski, West Des Moines, IA (US); Lan Zhou, Urbandale, IA (US)

Correspondence Address: ALSTON & BIRD LLP;PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignees: Pioneer Hi-Bred International, Inc., Johnston, IA (US); E.I. duPont de Nemours and Company, Wilmington, DE (US)

(21) Appl. No.: 11/021,666

(22) Filed: Dec. 23, 2004

Related U.S. Application Data
(60) Provisional application No. 60/532,196, filed on Dec. 23, 2003.

(51) Int. Cl. A01H 1/00; C12N 15/82

(52) U.S. Cl. 800/281; 435/468

(57) ABSTRACT

Compositions and methods for modulating the production and characteristics of oil in a plant or plant part thereof are provided. Compositions of the invention are nucleotide constructs that provide for expression of transcriptional activators that increase lipid biosynthesis in a plant in combination with the inhibition or disruption of starch biosynthesis and/or starch storage, and optionally in combination with expression of another polynucleotide of interest. Compositions also include transformed plants, plant cells, plant tissues, and plant seeds comprising these nucleotide constructs. The methods of the invention comprise introducing a combination of polynucleotides into a plant, wherein the polynucleotides provide for increased lipid biosynthesis and disruption of starch biosynthesis and/or starch storage, and optionally alteration of fatty acid metabolism. The methods and constructs find use in alteration of oil phenotype in a plant or plant part thereof.

### CROSS-REFERENCE TO RELATED APPLICATIONS

This application claims the benefit of U.S. Provisional Application Ser. No. 60/532,196, filed Dec. 23, 2003, the contents of which are herein incorporated by reference in their entirety.

### FIELD OF THE INVENTION

The present invention is drawn to plant genetics and molecular biology. More particularly, the methods involve altering oil phenotype in plants by modulating the expression of nucleic acids in plants.

### BACKGROUND OF THE INVENTION

A major focus of crop plant breeders over the last fifty years has been the modification of traits that affect seed composition. Of particular interest to plant breeders have been traits that affect oil content of the seed. The efforts of plant breeders have led to the development and introduction of crop plants with modified levels of oil. Additionally, plant breeders have developed cultivars with modified oil. For example, plant breeders have developed low-erucic-acid varieties of rapeseed (Brassica sp.). One of the most important components of seeds is oil because this component is used for human and livestock consumption; therefore, crop improvement efforts will continue to focus on these components.

While mankind has derived many benefits from the past efforts of plant breeders, the combination of the rapidly increasing human population and the decline in land available for agriculture places a tremendous burden on agriculturists to increase both agricultural output and productivity. New and improved crop plant varieties are desired by agriculturists to help meet the challenge of feeding the world's human population. Although traditional plant breeding approaches for crop plant improvement have been successful, the traditional approaches are slow and limited to naturally occurring genetic variation or artificially induced mutations. To keep pace with the escalating demands that the increase in the world's population places on agriculture, more rapid approaches for developing crop plants are necessary. In particular, methods are needed to modify the oil characteristics of plants.

### BRIEF SUMMARY OF THE INVENTION

Compositions and methods are provided for modulating the production and characteristics of oil in a plant or plant tissue. Compositions of the invention include nucleotide constructs that provide for the expression of transcriptional activators capable of increasing lipid biosynthesis in combination with expression of inhibitory sequences that disrupt starch biosynthesis and/or storage, and optionally in combination with expression of an additional polynucleotide of interest. In some embodiments, the additional polynucleotide of interest comprises a sequence that is capable of modifying saturation of lipids within the cell. In one combination, the nucleotide constructs of the invention comprise a polynucleotide encoding the transcriptional activator LEC1, a polynucleotide comprising an inhibitory sequence for AGP, and, optionally, a polynucleotide comprising an inhibitory sequence for FAD2. Compositions of the invention also include vectors, transformed plants, plant cells, plant tissues, and plant seeds comprising these nucleotide constructs.

Methods of the invention provide for the expression of transcriptional activators that affect lipid biosynthesis in a plant in combination with the inhibition or disruption of starch biosynthesis and/or starch storage. Optionally, additional sequences may be expressed that affect oil production or characteristics, such as the oleic acid content, the linoleic acid content, and the like. The methods result in a dramatic increase in oil production. The sequences of interest may be introduced independently into plant lines and crossed to obtain a plant having the combination of increased production of transcriptional activators and decreased starch biosynthesis. Alternatively, constructs may be stacked on the same plasmid for transformation or by repeated transformation of a previously transformed line. Also included are methods of transformation and regeneration of plants comprising the novel nucleotide constructs.

These compositions and methods are useful for improving oil production and characteristics in commercially important plants and plant parts. For instance total oil content can be increased in seed and seed embryos. Further, the level of desirable oils can be increased while the level of undesirable oils can be decreased. Thus, the compositions and methods can be used to improve the yield efficiency of desirable plant products.

### BRIEF DESCRIPTION OF THE DRAWINGS

FIG. 1 depicts the average percentage of change in oil concentration of transformed embryos compared to wild-type embryos.

FIG. 2 depicts the average percentage of change in oil concentration of transformed embryos compared to wild-type embryos.

FIG. 3 depicts the average oil concentration (mg/embryo) in wild-type and transformed embryos.

FIG. 4 depicts the average oil concentration (grams oil/100 grams embryo dry weight) of wild-type and transformed embryos.

FIG. 5 depicts the average percentage of change in embryo dry weight of transformed embryos compared to wild-type embryos.

FIG. 6 depicts a vector (plasmid A) carrying both an AGP inhibitory sequence (SEQ ID NO:15 comprising a fusion of a truncated AGP2 cosuppressive sequence and an AGP1 cosuppressive sequence) under control of the oleosin (OLE) promoter and a LEC1 coding sequence under the control of the LTP2 promoter.

FIG. 7 depicts the amino acid sequence for AGP2 compared to a truncated amino acid sequence for AGP2.

FIG. 8 depicts a vector (plasmid B) carrying two LEC1 coding sequences under control of the LTP2 promoter and a gamma zein promoter (GZ-W64a), respectively.

FIG. 9 depicts a vector (plasmid C) comprising a LEC1 expression construct (comprising the LEC1 coding sequence of SEQ ID NO:1 driven by the LTP2 promoter), an AGP2 inhibitory construct (comprising a truncated AGP2 sequence and an its complement (sequence shown in SEQ ID NO:16) driven by the OLE promoter), and an FAD2 inhibitory construct (comprising one truncated FAD2 region, an intervening FAD2 sequence, and the complement of the truncated FAD2 region (sequence shown in SEQ ID NO:17) driven by the embryo abundant protein (EAP) promoter).

FIG. 10 depicts a vector (plasmid D) comprising a LEC1 expression construct (comprising the LEC1 coding sequence of SEQ ID NO:1 driven by the LTP2 promoter) and a dual AGP2-FAD2 inhibitory construct (comprising a fusion sequence (SEQ ID NO:18) having two truncated FAD2 sequences and their complements, as well as one truncated AGP2 sequence and its complement) driven by the OLE promoter).

FIG. 11 depicts a linear map of AGP2 and FAD2 inhibitory sequence regions for plasmids C and D shown in FIGS. 9 and 10, respectively.

FIG. 12 depicts the oleic acid (C18:1) values as a percentage of total fatty acids for seed transformed with plasmid C (comprising the LEC1 coding sequence (SEQ ID NO:1) operably linked to the LTP2 promoter, an AGP2 inhibitory sequence (SEQ ID NO:16) operably linked to the OLE promoter, and an FAD2 inhibitory sequence (SEQ ID NO:17) operably linked to the EAP promoter).

FIG. 13 depicts the oleic acid (C18:1) values as a percentage of total fatty acids for seed transformed with plasmid D (comprising the LEC1 coding sequence (SEQ ID NO: 1) operably linked to the LTP2 promoter, and the fusion AGP2-FAD2 inhibitory sequence (SEQ ID NO:18) operably linked to the OLE promoter).

FIG. 14 depicts the oleic acid (C18:1) percentage of total fatty acids as a function of embryo AGPase concentration (mUnits/mg of dry weight) for seed transformed with plasmid C (comprising the LEC1 coding sequence (SEQ ID NO:1) operably linked to the LTP2 promoter, an AGP2 inhibitory sequence (SEQ ID NO:16) operably linked to the OLE promoter, and an FAD2 inhibitory sequence (SEQ ID NO:17) operably linked to the EAP promoter).

FIG. 15 depicts the oleic acid (C18:1) percentage of total fatty acids as a function of embryo AGPase concentration (mUnits/mg of dry weight) for seed transformed with plasmid D (comprising the LEC1 coding sequence (SEQ ID NO:1) operably linked to the LTP2 promoter, and the fusion AGP2-FAD2 inhibitory sequence (SEQ ID NO:18) operably linked to the OLE promoter).

FIG. 16 depicts the total oil concentration (percentage of dry weight) as a function of embryo AGPase concentration (mUnits/mg of dry weight) for seed transformed with plasmid C (comprising the LEC1 coding sequence (SEQ ID NO:1) operably linked to the LTP2 promoter, an AGP2 inhibitory sequence (SEQ ID NO:16) operably linked to the OLE promoter, and an FAD2 inhibitory sequence (SEQ ID NO:17) operably linked to the EAP promoter).

FIG. 17 depicts the total oil concentration (percentage of dry weight) as a function of embryo AGPase concentration (mUnits/mg of dry weight) for seed transformed with plasmid D (comprising the LEC1 coding sequence (SEQ ID NO:1) operably linked to the LTP2 promoter, and the fusion AGP2-FAD2 inhibitory sequence (SEQ ID NO:18) operably linked to the OLE promoter).

FIG. 18 depicts the ratio of the oil concentration (mg) of seed as a percentage of control for embryos transformed with various vectors: plasmid C (comprising the LEC1 coding sequence (SEQ ID NO:1) operably linked to the LTP2 promoter; an AGP2 inhibitory sequence (SEQ ID NO:16) operably linked to the OLE promoter; and an FAD2 inhibitory sequence (SEQ ID NO:17) operably linked to the EAP promoter); plasmid D (comprising the LEC1 coding sequence (SEQ ID NO:1) operably linked to the LTP2 promoter; and the fusion AGP2-FAD2 inhibitory sequence (SEQ ID NO:18) operably linked to the OLE promoter); plasmid E (comprising the LEC1 coding sequence (SEQ ID NO:1) operably linked to the LTP2 promoter; and an FAD2 inhibitory sequence operably linked to the OLE promoter, which expresses a cosuppression inhibitor of FAD2 in the sense orientation); and plasmid F (comprising the LEC1 coding sequence (SEQ ID NO:1) operably linked to the LTP2 promoter; and an FAD2 inhibitory sequence (SEQ ID NO:17) operably linked to the OLE promoter).

### DETAILED DESCRIPTION OF THE INVENTION

The present invention is drawn to methods and compositions for altering oil production and oil traits by modifying both lipid and starch metabolism in plants or plant parts thereof. The methods comprise the use of specific combinations of nucleotide constructs to manipulate these two biosynthetic pathways such that carbon partitioning is shifted away from starch synthesis in favor of lipid synthesis, the net result of which is an increase in oil content in a plant or plant part thereof. While manipulation of either of these pathways in isolation is known to affect oil production, introduction of the combination of polynucleotides disclosed herein produces unexpected results not previously predicted. See copending U.S. Provisional Patent Application Ser. No. 60/532,196, filed Dec. 23, 2003 and entitled “Alteration of Oil Traits in Plants,” herein incorporated by reference in its entirety.

In particular embodiments, oil production is enhanced by increasing expression of transcriptional activators that contribute to lipid biosynthesis in combination with disrupting starch biosynthesis, disrupting storage of starch, or disrupting both starch biosynthesis and storage of starch. Of particular interest is the use of one or more nucleotide constructs that provide for expression of transcriptional activators, for example, leafy cotyledon 1 transcriptional activator (LEC1) and others disclosed herein below, in conjunction with expression of inhibitory sequences that target one or more enzymes involved in starch biosynthesis, for example, inhibitory sequences targeting ADP-glucose pyrophosphorylase (AGP) activity. Suitable nucleotide constructs are provided herein for use in the methods of the invention. The compositions and methods of the invention find use in agriculture, particularly in the development of plant varieties with high oil production.

In accordance with the methods of the present invention, lipid metabolism and starch metabolism can be manipulated at various points within these metabolic pathways using any strategy known in the art so long as the end result, i.e., increased lipid synthesis and decreased starch synthesis, is achieved. Such strategies include, but are not limited to one or more of the following:
a) inhibiting expression or function of a gene product that is involved in starch biosynthesis or lipid catabolism;
b) increasing expression of a gene product that contributes to starch catabolism or lipid biosynthesis;
c) modifying activity of an enzyme that is involved in starch or lipid metabolism;
d) modifying a protein that contributes to nucleating starch biogenesis;
e) modifying a protein that is involved in transport of assimilates that feed into the starch biosynthetic pathway;
f) eliminating a gene that is involved in starch metabolism or lipid catabolism;
g) mutation of a gene to effect reduced expression or activity of an enzyme that is involved in starch biosynthesis or mutation of a gene to effect increased expression or activity of an enzyme that is involved in lipid biosynthesis; and
h) shunting carbon away from a pathway for starch biosynthesis, thereby depriving the pathway of substrates for starch biosynthesis.

The terms “inhibit,” “inhibition,” and “inhibiting” as used herein refer to any decrease in the expression or function of a target gene product, including any relative decrement in expression or function up to and including complete abrogation of expression or function of the target gene product. The term “expression” as used herein in the context of a gene product refers to the biosynthesis of that gene product, including the transcription and/or translation of the gene product. Inhibition of expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, inhibition of expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product.

The term “inhibitory sequence” encompasses any polynucleotide or polypeptide sequence that is capable of inhibiting the expression of a target gene product, for example, at the level of transcription or translation, or which is capable of inhibiting the function of a target gene product. Examples of inhibitory sequences include, but are not limited to, full-length polynucleotide or polypeptide sequences, truncated polynucleotide or polypeptide sequences, fragments of polynucleotide or polypeptide sequences, variants of polynucleotide or polypeptide sequences, sense-oriented nucleotide sequences, antisense-oriented nucleotide sequences, the complement of a sense- or antisense-oriented nucleotide sequence, inverted regions of nucleotide sequences, hairpins of nucleotide sequences, double-stranded nucleotide sequences, single-stranded nucleotide sequences, combinations thereof, and the like. The term “polynucleotide sequence” includes sequences of RNA, DNA, chemically modified nucleic acids, nucleic acid analogs, combinations thereof, and the like.

Inhibitory sequences are designated herein by the name of the target gene product. Thus, for example, an “AGP inhibitory sequence” would refer to an inhibitory sequence that is capable of inhibiting the expression of AGP (ADP-glucose pyrophosphorylase), for example, at the level of transcription and/or translation, or which is capable of inhibiting the function of AGP. Similarly, an “FAD inhibitory sequence” would refer to an inhibitory sequence that is capable of inhibiting the expression of a fatty acid desaturase (for example, an FAD2 inhibitory sequence targeting FAD2), at the level of transcription and/or translation, or which is capable of inhibiting the function of an FAD. When the phrase “capable of inhibiting” is used in the context of a polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded RNA polynucleotides), or encodes an inhibitory polypeptide (i.e., a polypeptide that inhibits expression or function of the target gene product), following its transcription (for example, in the case of an inhibitory sequence encoding a hairpin RNA, miRNA, or double-stranded RNA polynucleotide) or its transcription and translation (in the case of an inhibitory sequence encoding an inhibitory polypeptide), the transcribed or translated product, respectively, exerts the inhibitory effect on the target gene product (i.e., inhibits expression or function of the target gene product).

Conversely, the terms “increase,” “increased,” and “increasing” in the context of the methods of the present invention refer to any increase in the expression or function of a gene product, including any relative increment in expression or function. As with inhibition, increases in the expression or function of a gene product of interest (i.e., a target gene product) can be in the context of a comparison between any two plants, for example, expression or function of a target gene product in a genetically altered plant versus the expression or function of that target gene product in a corresponding wild-type plant. Alternatively, increases in the expression or function of the target gene product can be in the context of a comparison between plant cells, organelles, organs, tissues, or plant parts within the same plant or between plants, and includes comparisons between developmental or temporal stages within the same plant or between plants. Any method or composition that up-regulates expression of a target gene product, either at the level of transcription or translation, or up-regulates functional activity of the target gene product can be used to achieve increased expression or function of the target gene product. For example, a method that increases oil production in a plant can be any method that increases the total oil content or the percent oil content of that plant relative to that observed in another plant, for example a comparison between a genetically modified plant and a corresponding wild-type plant, any method that increases oil content of a cell, organelle, organ, tissue, or plant part relative to a different cell, organelle, organ, tissue, or plant part within the same plant or between two plants, or any method that increases oil content of a cell, organelle, organ, tissue, plant part, or whole plant relative to that observed during different developmental or temporal stages within the same plant or another plant.

Recombinant DNA and plant transformation technologies offer a broad choice of mechanisms for inhibiting starch biosynthesis and/or starch storage and increasing lipid biosynthesis in order to obtain a genetically modified plant having the desired altered oil phenotype disclosed herein. By “phenotype,” it is intended any expressed characteristic or trait in an organism. Generally, the inhibition is achieved by introducing polynucleotides that are capable of diminishing or abolishing the activity of enzymes important, directly or indirectly, to starch biosynthesis and lipid (i.e., oil) catabolism or by like modulation of proteins important in the transport of assimilates required for starch production and lipid catabolism. Alternatively, the introduced polynucleotides can target activity of enzymes important, directly or indirectly, to starch degradation and lipid biosynthesis such that lipid biosynthesis is favored over starch biosynthesis. In some embodiments, the introduced polynucleotides provide for increased expression of transcriptional activators to effect increased lipid production, and decreased expression and/or function of ADP-glucose pyrophosphorylase (AGP) to effect decreased starch production, optionally in combination with decreased expression and/or function of one or more fatty acid desaturases (FADs) to effect altered oil traits. However, any of the methods below can be used, alone or in combination, to achieve a net shunting of carbon toward lipid biosynthesis, so long as at least one of the genetic manipulations described herein below ultimately resulted from one or more plant transformation events wherein one or more of the polynucleotides of interest described herein are introduced into the plant for which the altered oil phenotype is desired.

(A) Inhibition of Gene Products Involved in Starch or Oil Metabolism.

In some embodiments, the methods of the present invention objectively inhibit expression or function of a gene product that is involved in starch or lipid metabolism. In this manner, a decrease in starch synthesis can be achieved through reduced expression of genes that encode proteins that normally serve as starch nucleating proteins, enzymatic catalysts, or assimilate transporters involved in starch biosynthesis. Any of the mechanisms known in the art to effect inhibition of gene expression can be used to optimally interfere with starch biosynthesis genes including, but not limited to, genes encoding sucrose synthase, hexokinase(s), phosphoglucomutase, phosphoglucoisomerase, ADP-glucose pyrophosphorylase (AGP), amylogenin (a protein primer of starch biogenesis), soluble and bound starch synthase and starch branching enzymes, starch debranching enzymes, isoamylase enzymes, starch phosphorylases, and the Brittle-1 transport protein.

Alternatively, or in combination, an increase in lipid synthesis can be achieved through reduced expression of genes that encode proteins that normally catabolize oil and oil product intermediaries. Beta-oxidation is responsible for the catabolism of fatty acids to produce succinate for the production of sucrose. Fatty acyl-CoA is the substrate for fatty acid beta-oxidation in peroxisomes and mitochondria, which consists of four steps. The first step of beta-oxidation is catalyzed by Acyl-CoA oxidase. The second and third steps are catalyzed by a single enzyme, which has both enoyl-CoA hydratase and beta-hydroxyacyl-CoA dehydrogenase activities. The fourth step is catalyzed by 3-ketoacyl-CoA thiolase. The acetyl-CoA produced as a result of beta-oxidation is metabolized further to produce succinate by the glyoxylate pathway. Any of the mechanisms known in the art to effect inhibition of gene expression can be used to optimally interfere with lipid catabolism genes including, but not limited to, phospholipase A, C, and D (see, Wang (2001) Annu. Rev. Plant Physiol. Plant Mol. Biol. 52: 211-231), acyl-CoA oxidase (see, U.S. Pat. No. 6,489,461), and lipoxygenase (see, Siedow (1991) Annu. Rev. Plant Physiol. Plant Mol. Biol. 42: 145-188).

Methods for inhibiting gene expression are well known in the art and include, but are not limited to, homology-dependent gene silencing, antisense technology, RNA interference (RNAi), and the like. The general term homology-dependent gene silencing encompasses the phenomenon of cis-inactivation, trans-inactivation, and cosuppression. See Finnegan et al. (1994) Biotech. 12: 883-888; and Matzke et al. (1995) Plant Physiol. 107: 679-685; both incorporated herein in their entirety by reference. These mechanisms represent cases of gene silencing that involve transgene/transgene or transgene/endogenous gene interactions that lead to reduced expression of protein in plants. A “transgene” is a recombinant DNA construct that has been introduced into the genome by a transformation procedure. As one alternative, incorporation of antisense RNA into plants can be used to inhibit the expression of endogenous genes and produce a functional mutation within the genome. The effect is achieved by introducing into the cell(s) DNA that encodes RNA that is complementary to the sequence of mRNA of the target gene. See e.g. Bird et al. (1991) Biotech and Gen. Eng. Rev. 9: 207-226; incorporated herein in its entirety by reference. Manipulation of plant gene expression by homology-dependent gene silencing, antisense RNA, RNAi, or other inhibitory mechanism can be used to optimally interfere with starch biosynthesis or lipid catabolism, thereby resulting in increased oil production within the plant, or a desired plant part thereof. See also the more detailed discussion herein below addressing these and other methodologies for achieving inhibition of expression or function of a target gene product of interest.

Though the embodiments disclosed herein outline methods in which AGP activity is inhibited to disrupt starch biosynthesis, alone or in combination with inhibition of an FAD, for example, FAD2, it is recognized that other enzymes and proteins that contribute to starch biosynthesis and/or storage can be targeted in conjunction with methods for increasing lipid biosynthesis as noted elsewhere herein to achieve the overall objective, i.e., manipulating these two biosynthetic pathways in a plant or particular plant part thereof to favor oil production.

(B) Increased Expression of Gene Products Involved in Starch Catabolism and/or Increased Expression of Gene Products Involved in Lipid Synthesis.

Oil accumulation within a plant tissue or plant part of interest, for example, embryonic tissue within a seed, can be increased by altering the net distribution of carbon assimilate from deposition as a starch reserve to deposition as oil instead. One mechanism useful in achieving this end is to ensure that there is little or no net accumulation of carbon assimilate into starch, thereby providing these assimilates for the synthesis and deposition of oil. This outcome can be achieved by providing enhanced activity of starch degradative enzymes in the tissue or plant part targeted for increased oil accumulation. A number of such enzymes and corresponding isoenzymes are known to a skilled artisan. These enzymes can attack starch or carbohydrate components that may function as precursors of starch molecules and granules, and degrade the material down into intermediary forms that could funnel into mainstream metabolism and be used for the biosynthesis of stored lipids. See, e.g., Steup (1990) in Methods in Plant Biochemistry, Vol. 3 (Academic Press, NY), pp. 103-128; incorporated herein in its entirety by reference. Examples of enzymes that could be expressed in transgenic plants and lead to increased rates of starch degradation (or instability of starch-forming molecules) include, but are not limited to, alpha-amylase, beta-amylase, alpha-glucosidases, and starch phosphorylase.

Where carbon is freed up for shunting into lipid biosynthesis, increased oil production can be favored by increased expression of proteins that are involved in the lipid anabolic pathway. Such sequences include, but are not limited to, SLC-1, the sn-2 acyl transferase gene (see Zou et al. (1997) Plant Cell 9: 909-923), ACCase, acetyl-CoA carboxylase (see Harwood et al. (1988) Annu. Rev. Plant Physiol. Plant Mol. Biol. 39: 101-138; Beisson et al. (2003) Plant Physiol. 132: 681-697, and U.S. Pat. No. 5,925,805), acyl carrier protein (see U.S. Pat. No. 5,315,001), thioesterase (see U.S. Pat. No. 5,147,792), ketoacyl-synthases (see U.S. Pat. No. 6,348,642), malonyl transacylase, stearoyl-ACP desaturase, and the like. MAP kinase 3, receptor-like protein kinase, calcium EF-hand protein and ATP citrate lysase are members of protein families that also influence oil accumulation in plants (see U.S. Patent Application Publication No. 20030204870, herein incorporated by reference in its entirety). Of particular interest are transcriptional activators that function to increase the lipid anabolic pathway. Suitable transcriptional activators include, but are not limited to, HAP (heme-activated protein)3-type CCAAT box binding transcriptional activators, particularly the leafy cotyledon 1 transcriptional activator (LEC1), the HAP2 transcriptional activators, the HAP5 transcriptional activators, the receptor-like protein kinases, MAPKK3/MEK3 (MAP kinase kinase) transcriptional activators, and the LIP15, SNF1, and CKC Aintegumenta transcriptional activators as noted herein below.

(C) Modification of the Activity of Enzymes Responsible for Starch and/or Lipid Metabolism.

One of the predominant ways in which enzyme activity is controlled in cells is by allosteric regulation in which regulatory metabolites bind to regulatory sites on the enzyme. The binding of substrate to one active site can affect the properties of other active sites in the same enzyme molecule. Allosteric regulation by small molecules is profoundly significant in controlling enzymatic activity. For example, two allosteric enzymes that are well understood are the bacterial phosphofructikinase and aspartate transcarbamoylase. See, for example, Creighton (1993) Proteins: Structures and Molecular Properties (2^{nd} ed.; Freeman & Co.), pp. 444-452; incorporated herein in its entirety by reference.

ADP-glucose pyrophosphorylase (AGP) is an allosteric enzyme. In plants it is activated by 3-phosphoglyceric acid (“3-PGA”) and inhibited by inorganic phosphate (Pi) to varying degrees depending upon the species and organ source. See, for example, Preiss (1993) Denpun Kagaku 40: 117-131 and Cross et al. (2004) Plant Physiol. 135: 137-144; both incorporated herein in their entirety by reference.

The importance of the allosteric properties of AGP has recently been demonstrated when transgenic plants expressing a bacterial form of AGP (glgC16), which is allosterically deregulated compared to the native plant AGP, accumulated 35% more starch than controls. See, Stark et al. (1992) Science 258: 287-292, incorporated herein in its entirety by reference. Furthermore, allosteric modification of the maize native Sh2 gene can increase seed weight, presumably due, at least in part, to an effect on starch deposition. See, Giroux et al. (1996) Proc. Natl. Acad. Sci. USA 93: 5824-5829; incorporated herein in its entirety by reference. Allosteric variants of AGP, which are less responsive to 3-PGA and Pi occur naturally in plants. See, Kleczkowsi et al. (1993) Plant Physiol. 101: 179-186; incorporated herein in its entirety by reference. These variants can also be engineered into plants through gene manipulation and plant transformation.

As mentioned above, the reduction of AGP activity in the cell, either plastidic and/or cytoplasmic (Kim et al. (1989) Plant Physiol. 91: 217-220; and Miller et al. (1995) Planta 197: 522-527; all of which are incorporated herein in their entirety by reference) will also decrease starch synthesis. This can be accomplished by the absence or reduced function of either of two subunits that normally interact to form the active holoenzyme. However, this enzyme may also be exploited by way of its allosteric properties. ADP-glucose pyrophosphorylase activity is inhibited by Pi. If the subunit(s) of the enzyme is rendered more sensitive to Pi through molecular and protein engineering, then expression of this modified gene would produce a protein that competes with the native subunit to form a holoenzyme. The activity of AGP could be reduced proportionally to that fraction of holoenzyme that contains the subunit rendered more sensitive to Pi. Allosteric modification of AGP activity can also be accomplished through re-dox modification, see for example Hendriks et al. (2003) Plant Physiol. 133(2): 838-849, herein incorporated in its entirety by reference.

Alternatively, genes that express enzymes or regulate enzymes in oil anabolism can increase oil synthesis in a plant cell. As described above, genes such as SLC-1, ACCase, and LEC1 all increase oil synthesis levels when over expressed in a cell. Increasing the functional activity of any gene involved in the lipid anabolic pathway using any of the methods disclosed herein increases oil production in plants.

(D) Modification of a Protein Involved in Nucleating Starch Biogenesis.

The protein FtsZ, serves a role in regulating starch granule and size. See, for example, International Patent Application No. WO 03/035874, incorporated herein in its entirety by reference. Starch synthesis can also be diminished by modifying the initiator system, which primes the glucose polymerization process. A self-glucosylating protein may act in a nucleating function for the biogenesis of starch molecules. Such is the case in glycogen biogenesis. See, Lomako et al. (1988) FASEB Journal 2: 3097-3103; incorporated herein in its entirety by reference. Elimination of proteins involved in starch nucleation, or formation of a non-functional homolog and expression in the cells, leads to interruption of starch biosynthesis and increased accumulation of oil.

(E) Modification of a Protein Responsible for Transport of Assimilates that Channel into Starch Biosynthesis.

Proteins involved in assimilate transport, such as the Brittle-1 protein of maize endosperm amyloplast membranes, sucrose carrier proteins or others homologous to the hexose transporter of the chloroplast, or plastidic ATP/ADP-transporter proteins (Geigenberger et al. (2001). Plant Physiol 125(4): 1667-1678) can also affect starch synthesis by restricting the availability of substrates for normal starch and/or sugar metabolism. See, Cao et al. (1995) Plant, Cell Environ. 18: 1019-1026; and Weig et al. (1996) J Plant Physiol. 147(6): 685-69; both incorporated herein in their entirety by reference.

The present proteins may also be targeted for disruption of starch synthesis. Included are proteins serving as transporters of intermediates upon which starch synthesis depends. Absence of the Brittle-1 protein in maize causes a severe reduction in starch deposition within the endosperm. This protein acts to transport sugar nucleotide molecules into the amyloplast where starch is made. See, Boyer et al. (1994) in Specialty Corns, ed. A Hallauer (Boca Raton CRC Press, Inc.), pp. 1-28; and Shannon et al. (1996) Plant Physiol. 110(3): 835-843; both incorporated herein in their entirety by reference.

Knock-out of the Brittle-1 homologue in maize germ or dicotyledonous plants, for example, could result in a similar interruption of starch synthesis in these tissues. Alternatively, over expression of transporter proteins delivering substrates into the lipid biosynthetic pathway can increase oil production.

(F) Mutation Restriction and Mutants with Altered Enzyme Activity.

Elimination of gene action and starch deposition through mutation restriction of starch biosynthesis in an oil-bearing organ may be achieved in many ways. One may select for a mutant that produces less starch in the tissue of interest, assuming that the tissue is normally enriched in oil. Such mutants can be generated in seed in many ways known to the skilled artisan, such as chemical mutagenesis, x-ray-induced mutagenesis, transposon-induced mutagenesis or mutagenesis by natural means. See, Denyer et al. (1995) Plant Cell Environ. 18: 1019-1026; incorporated herein in its entirety by reference. Mutants that produce less starch can be identified by different methods known in the art. Oil concentration in such mutants is higher than that found in comparable plant material of a non-mutagenic state.

(G) Shunting Carbon away from Starch Biosynthesis by Denying Pathway of Substrate to Achieve Enhanced Accumulation of Oil in Oil Bearing Crops.

During seed development of commercial oilseeds crops (e.g., canola, soybean and sunflower), soluble sugars and starch are significant constituents of the developing cotyledons. For example, it has been shown with sunflowers grown in the field that starch concentration in developing seed can be between 5% and 23% of dry matter. See, Luthra et al. (1991) J Plant Physiol. 137: 312-318; incorporated herein in its entirety by reference.

The exact concentration is dependent upon the stage of development and genotype, as starch concentration reaches a peak in early to mid-development and then declines to a low level as triacylglycerol accumulation becomes a primary activity in the seed. A similar phenomenon occurs for starch concentration in developing seed of soybean (Yazdi-Samadi, R, Rinne et al. (1977) Agron. J 69: 481-486; incorporated herein in its entirety by reference) and canola (see, Norton et al. (1975) Planta Berl. 123: 163-174; incorporated herein in its entirety by reference. By specific gene inactivation (e.g. by cosuppression, antisense, RNAi, etc.) of ADP-glucose pyrophosphorylase activity in the cotyledons of these plants, starch accumulation is severely reduced. In turn, carbon is made available for alternate partitioning within the cells of the seed. Because triacylglycerol biosynthesis requires large inputs of carbon assimilates and is an active and primary metabolic function during seed development, carbon assimilates are metabolically shuttled away from starch synthesis and into the enhanced production of oil materials.

Thus, a number of strategies can be employed to achieve the objective of the methods of the present invention, i.e., alteration of starch and lipid metabolism such that carbon is shunted away from starch synthesis and/or starch storage into lipid biosynthesis, resulting in increased oil production in a plant or plant part of interest and an attendant alteration of oil phenotype. Of particular interest to the present invention are strategies that combine the approaches of enhancing expression or function of proteins that are involved in lipid biosynthesis, more particularly, suitable transcriptional activators described herein, and inhibiting expression or function of proteins that are involved in starch synthesis.

Thus, in some embodiments of the invention, increased oil content is achieved by introducing into a plant of interest a combination of polynucleotides, where the combination of polynucleotides provides for an increase in lipid biosynthesis in conjunction with disruption of starch biosynthesis and/or disruption of storage of starch as described herein below. Where additional modification of one or more agronomic traits is desirable, the methods of the invention further comprise introducing into the plant one or more other polynucleotides that provide for the additional modification(s) of the plant's agronomic traits. In some embodiments, the additional polynucleotide(s) provide(s) for alteration of the quality of the increased oil produced within the plant or plant part thereof. The combination of polynucleotides can be introduced into the plant of interest using any method known to those of skill in the art, including the transformation and plant breeding methods noted elsewhere herein. As previously noted, at least one of the polynucleotides is introduced via a plant transformation event, for example, using a transformation method described herein below. Further, the combination of polynucleotides can be introduced as part of a single nucleotide construct; alternatively, the combination of polynucleotides can be introduced as multiple nucleotide constructs, each comprising one or more of the polynucleotides of interest for introduction into a plant of interest. The invention further provides compositions that are nucleotide constructs for use in practicing the methods newly disclosed herein.

In this manner, a first polynucleotide comprising a sequence encoding a protein that is capable of increasing lipid biosynthesis, and which is operably linked to a promoter that drives expression in a plant cell, is introduced into the plant of interest in combination with a second polynucleotide comprising a sequence that is capable of disrupting starch biosynthesis and/or disrupting storage of starch. The first polynucleotide can encode any protein the expression of which results in increased lipid biosynthesis. Examples of such proteins include, but are not limited to, proteins that are involved in lipid anabolic processes, for example, SLC-1, the sn-2 acyl transferase gene (see Zou et al. (1997) Plant Cell 9: 909-923), ACCase, acetyl-CoA carboxylase (see Harwood et al. (1988) Annu. Rev. Plant Physiol. Plant Mol. Biol. 39: 101-138; Beisson et al. (2003) Plant Physiol. 132: 681-697, and U.S. Pat. No. 5,925,805), acyl carrier protein (see U.S. Pat. No. 5,315,001), thioesterase (see U.S. Pat. No. 5,147,792), ketoacyl-synthases (see U.S. Pat. No. 6,348,642), malonyl transacylase, stearoyl-ACP desaturase, and the like. MAP kinase 3, receptor-like protein kinase, calcium EF-hand protein and ATP citrate lysase are members of protein families that also influence oil accumulation in plants (see U.S. Patent Application Publication No. 20030204870, herein incorporated by reference in its entirety).

Of particular interest are transcriptional activators that function to increase the lipid anabolic pathway. Suitable transcriptional activators include, but are not limited to, HAP (heme-activated protein)3-type CCAAT box binding transcriptional activators, particularly the leafy cotyledon 1 transcriptional activator (LEC1), the HAP2 transcriptional activators, the HAP5 transcriptional activators, the receptor-like protein kinases, and MAPKK3/MEK3 (MAP kinase kinase) transcriptional activators. See, for example, U.S. Pat. No. 6,825,397; U.S. Patent Application Publication No. 20030167526, entitled “Compositions and Methods for Identifying Transformed Cells”; U.S. Patent Application Publication No. 2003126638; U.S. Patent Application Publication No. 2003204870; U.S. Patent Application No. 20040016022; and WO 02/057439, herein incorporated by reference in their entirety. Other transcriptional activators capable of increasing lipid biosynthesis include LIP15, SNF1, and CKC Aintegumenta (see U.S. Patent Application Publication No. 20030204870, entitled “Alteration of Oil Traits in Plants,” herein incorporated by reference in its entirety).

The suffix “-like” added to any named nucleic acid or amino acid sequence of the aforementioned families refers to additional members of the respective families that share sequence identity as disclosed herein and/or functionality with the nucleotide sequences and the corresponding amino acid sequences encoded by such nucleotide sequences disclosed in the present invention. Additionally, it may be useful in the practice of the invention to combine such sequences in the same plant. For example, lipid synthesis can be increased by introducing polynucleotides encoding LEC1, SLC, and ACCase together into the same plant.

The first polynucleotide is introduced into the plant of interest in combination with a second polynucleotide that is capable of disrupting starch biosynthesis and/or disrupting storage of starch. By “disrupting starch biosynthesis” is intended any modification to the starch anabolic pathway that results in a net decrease in starch production. By “disrupting storage of starch” is intended any modification to the starch catabolic pathway that results in an increase in starch degradation and net decrease in starch accumulation. Disruption of starch biosynthesis and/or storage of starch can be achieved by any method known to those skilled in the art, including, but not limited to, the methods disclosed herein above.

In preferred embodiments, disruption of starch biosynthesis and/or starch storage is achieved via inhibition of expression or function of at least one of the following gene products: ADP-glucose pyrophosphorylase (AGP) or a portion thereof (i.e., AGP1 or AGP2) (see, for example, U.S. Pat. No. 6,232,529, herein incorporated by reference in its entirety); ADP-glucose pyrophosphatase (AGPPase) (Rodriguez-Lopez et al. (2000) Proc. Natl. Acad. Sci. 97: 8705-8710); UDP-glucose pyrophosphorylases; UDP-glucose protein transglucosylase; starch phosphorylases; isoamylases and disproportionating enzymes; sucrose synthases; sucrose phosphate synthase; sucrose phosphate phosphorylase; hexokinase(s); phophoglucomutase; phosphoglucoisomerase; soluble and bound starch synthase and starch branching enzymes; starch debranching enzymes; isoamylase enzymes; the Brittle-1 transport protein; and the like.

The second polynucleotide can comprise any inhibitory nucleotide sequence that is capable of disrupting starch biosynthesis and/or disrupting storage of starch, including nucleotide sequences that directly (i.e., do not require transcription) or indirectly (i.e., require transcription or transcription and translation) disrupt starch biosynthesis and/or storage of starch by targeting expression and/or function of one or more of the gene products that is involved in starch synthesis. For example, the second polynucleotide can comprise an inhibitory sequence that is could be a chemically synthesized or in vitro-produced small interfering RNA (siRNA) or micro RNA (miRNA) that, when introduced into the plant cell, tissue, or organ, would directly, though transiently, disrupt starch biosynthesis and/or storage of starch in the cell, tissue, or organ by silencing expression of the target gene product of interest. Alternatively, the second polynucleotide can comprise an inhibitory sequence that encodes an inhibitory nucleotide molecule that is designed to silence expression of the gene product of interest, such as sense-orientation RNA, antisense RNA, double-stranded RNA (dsRNA), hairpin RNA (hpRNA), intron-containing hpRNA, catalytic RNA, miRNA, and the like. In yet other embodiments, the second polynucleotide sequence can comprise an inhibitory sequence that encodes a mRNA, the translation of which yields a polypeptide of interest that inhibits expression or function of one or more of the gene products that is involved in starch synthesis. Where the second polynucleotide comprises an inhibitory sequence that encodes an inhibitory nucleotide molecule or a mRNA for a polypeptide of interest, the inhibitory sequence is operably linked to a promoter that drives expression in a plant cell so that the encoded inhibitory nucleotide molecule or mRNA can be expressed.

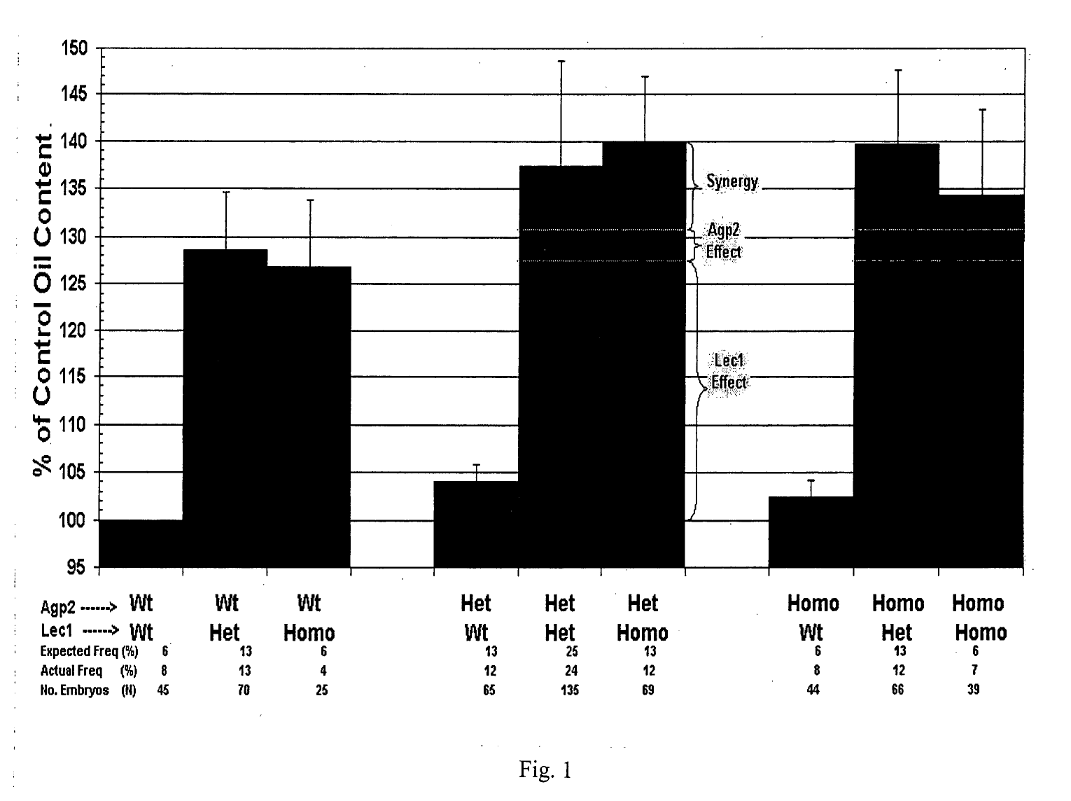

In this manner, the present invention provides some embodiments wherein oil content in a plant or plant part thereof is increased by increasing expression of the leafy cotyledon 1 transcriptional activator (LEC1) or functional variant or fragment thereof as described herein below in combination with inhibiting expression or function of AGP or portion thereof (i.e., AGP1 or AGP2). LEC1 is a transcriptional activator of the Hap3/Lec1 CCAAT box binding factors. LEC1 is well known in the art and is known to induce embryogenesis, as well as increase oil production in a plant. The family of LEC1 factors and functional fragments thereof share at least a region that corresponds to the consensus sequence shown in SEQ ID NO:14 (see, for example, U.S. Patent Application Publication No. 20030126638, Table 1 and FIG. 1; International Publication No. WO 00/28058 as shown in SEQ ID NO:23), and GenBank Accession No. AF410176 (Zea mays LEC1). Any sequence encoding LEC1 or functional variant or fragment thereof can be used to practice the methods of the present invention, including, but not limited to, sequences encoding, for example, Zea mays LEC1 (SEQ ID NO:2, encoded by SEQ ID NO:1; see also GenBank Accession No. AAK95562, encoded by the nucleotide sequence deposited as GenBank Accession No. AF410176).

ADP-glucose pyrophosphorylase (AGP) is a heterotetrameric protein having ADP-glucose pyrophosphorylase activity. AGP generally comprises two subunits of ADP-glucose pyrophosphorylase large subunit (AGP1) and two subunits of ADP-glucose pyrophosphorylase small subunit (AGP2). However, it is recognized in the art that AGP can be encoded by several genes such as those nucleotide sequences disclosed in U.S. Pat. No. 6,232,529 B1 and the GenBank Accession Nos. M81603 (Zea mays shrunken2), and AF334959 (Zea mays brittle-2), herein incorporated by reference in their entirety.

Numerous genes encoding the small and large subunits of AGP from plants have been described (Smith-White and Preiss (1992) J. Mol. Evol. 34: 449-464 and Giroux (1995) Plant Physiol. 108: 1333-1334; both incorporated herein in their entirety by reference). The corresponding genes that have been described for maize are the endosperm specific Bt2 and Sh2 genes (Bae et al (1990) Maydica 35: 317-322; Bhave et al. (1990) Plant Cell 2: 581-588; incorporated herein in its entirety by reference). Genes encoding small subunit AGP (AGP2) polypeptides of maize (sometimes termed Agpsemzm) are known in the art. See, Hannah et al. (2001) Plant Physiol. 127(1): 173-183, herein incorporated by reference in its entirety. Although referred to as an embryo isoform because of its predominance in the germ of the seed, AGP is also expressed in the endosperm (Giroux and Hannah (1994) Mol. Gen. Genet 243: 400-408; incorporated herein in its entirety by reference).

By interfering with expression and function of AGP, which generally functions in the starch anabolism pathway, it is known that oil production is increased simultaneously with decreases in starch production as disclosed in U.S. Pat. No. 6,232,529 B1. During seed development of commercial oilseed crops (e.g., canola, soybean, and sunflower) or development of embryonic and scutellar tissues in cereal crops (e.g., maize, rice, wheat, barley, sorghum), soluble sugars and starch are significant constituents of the developing organs. By specific inhibition of AGP activity in the cotyledons of these plants, starch accumulation can be severely reduced. In turn, carbon is made available for alternate partitioning within the cells of the seed. Because triacylglycerol biosynthesis requires large inputs of carbon assimilates and is an active and primary metabolic function during seed development, carbon assimilates are metabolically shuttled away from starch synthesis and into the enhanced production of oil materials.

Any inhibitory sequence can be utilized to inhibit AGP activity in the plant or specific plant part thereof. The inhibitory sequence can target the AGP heterotetrameric protein or any portion thereof, for example, the large subunit AGP1 or the small subunit AGP2. In this manner, a polynucleotide comprising an inhibitory sequence for AGP1, AGP2, or a combination thereof (i.e., both AGP1 and AGP2) is introduced into the plant of interest in combination with the polynucleotide comprising a sequence encoding the LEC1 transcriptional activator or functional fragment or variant thereof. As previously noted, the AGP inhibitory sequence can be a chemically synthesized or in vitro-produced small interfering RNA (siRNA) or micro RNA (miRNA) that, when introduced into the plant cell, tissue, or organ, would directly, though transiently, inhibit AGP1, AGP2, or a combination thereof by silencing expression of these target gene product(s). Alternatively, the AGP inhibitory sequence can encode an inhibitory nucleotide molecule that is designed to silence expression (i.e., targeting transcription and/or translation) of AGP1, AGP2, or a combination thereof, such as sense-orientation RNA, antisense RNA, double-stranded RNA (dsRNA), hairpin RNA (hpRNA), intron-containing hpRNA, catalytic RNA, miRNA, and the like. In yet other embodiments, the inhibitory sequence can encode a mRNA, the translation of which yields a polypeptide that inhibits expression or function of AGP1, AGP2, or a combination thereof. Where the polynucleotide comprises an inhibitory sequence that encodes an inhibitory nucleotide molecule that targets expression of AGP1, AGP2, or a combination thereof, or that encodes a mRNA for a polypeptide that inhibits expression or function of AGP1, AGP2, or a combination thereof, the inhibitory sequence is operably linked to a promoter that initiates transcription in a plant cell so that the encoded inhibitory nucleotide molecule or mRNA can be expressed.

Thus, in some embodiments of the invention, a polynucleotide encoding LEC1 or functional variant or fragment thereof is introduced into a plant of interest, for example, by stably incorporating into the genome of the plant a nucleotide construct comprising this polynucleotide operably linked to a promoter that is functional in a plant cell, and a second polynucleotide comprising an AGP inhibitory sequence is also introduced into the plant of interest, for example, by stably incorporating into the genome of the plant a nucleotide construct comprising this second polynucleotide. The introduced polynucleotides can be assembled as part of a single nucleotide construct, or as separate nucleotide constructs, and can be located on the same or different transformation vectors. Accordingly, the polynucleotides of interest can be introduced into the plant of interest in a single transformation event, in separate transformation events, or as part of a breeding protocol, so long as at least one plant transformation event has occurred in process of developing the plant having the desired characteristics of increased lipid synthesis and disrupted starch synthesis.

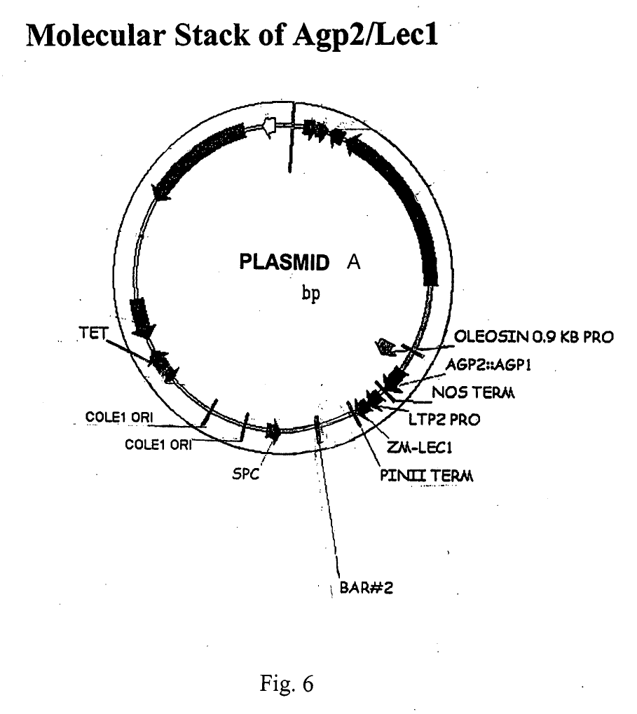

For example, in some embodiments, the first polynucleotide comprises a LEC1 nucleotide sequence set forth in SEQ ID NO:1 or GenBank Accession No. AF410176 (Zea mays LEC1) and the second polynucleotide comprises an AGP inhibitory nucleotide sequence such as the nucleotide sequence of GenBank Accession No. AY032604 (AGP2 small subunit nucleotide sequence, SEQ ID NO:3); the nucleotide sequence of GenBank Accession No. AY105915 (AGP2 small subunit nucleotide sequence); a nucleotide sequence encoding SEQ ID NO:4 (AGP2 small subunit amino acid sequence); SEQ ID NO:5 (AGP2 truncated nucleotide sequence); nucleotide sequences encoding SEQ ID NO:6 (AGP2 truncated amino acid sequence); the nucleotide sequence of GenBank Accession No. M81603 (Zea mays shrunken2); the nucleotide sequence of GenBank Accession No. AF334959 (Zea mays brittle2); the nucleotide sequence of GenBank Accession No. Z38111 (encoding AGP1, SEQ ID NO:7); the nucleotide sequence of GenBank Accession No. AY104549 (encoding AGP1); nucleotide sequences encoding SEQ ID NO:8 (AGP1 amino acid sequence); SEQ ID NO:9 (AGP1 truncated nucleotide sequence); nucleotide sequences encoding SEQ ID NO:10 (AGP1 truncated amino acid sequence), nucleotide sequences comprising a fusion of an AGP2 and AGP1 sequence or portions thereof (see, for example, the AGP2:AGP1 fusion sequence set forth in SEQ ID NO:15), or the like. In some embodiments, the first polynucleotide comprising a LEC1 encoding sequence operably linked to a promoter functional in a plant cell is within a nucleotide construct on a first vector (such as a plasmid) and the second polynucleotide comprising an AGP inhibitory sequence is within a nucleotide construct on a second vector. In some embodiments, more than one inhibitory sequence of AGP may be present, on the same or different vectors, such as an AGP1 inhibitory sequence and an AGP2 inhibitory sequence. In other embodiments, the nucleotide construct comprising the polynucleotide encoding LEC1 operably linked to a promoter that is functional in a plant cell is on the same vector as the nucleotide construct expressing the AGP inhibitory sequence or combinations of AGP inhibitory sequences (See FIG. 6).

The methods of the invention can additionally involve any method or mechanism known in the art for reducing or eliminating the activity or level of AGP in a cell including, but not limited to, antisense suppression, sense suppression, RNA interference, directed deletion or mutation, and dominant-negative strategies, and the like. Thus, the methods and compositions disclosed herein are not limited to any mechanism or theory of action and include any method where starch synthesis/storage is inhibited. For example, in some embodiments, the AGP inhibitory sequence (e.g., an AGP1 inhibitory sequence, an AGP2 inhibitory sequence, or a combination thereof) is expressed in the sense orientation (see for example, the fusion AGP2:AGP1 sequence of SEQ ID NO:15). In some embodiments, the sense-oriented transcripts cause cosuppression. In other embodiments, the sense-oriented expression of truncated AGP1 and/or AGP2 transcripts causes nonfunctional AGP protein subunits to be expressed and thus to bind to the normal subunits thereby inhibiting the AGP holozyme. Alternatively, the AGP inhibitory sequence or sequences (e.g., full-length sequences, truncated sequences, fragments of sequences, combinations thereof, and the like) can be expressed in the antisense orientation and thus inhibit endogenous AGP expression or function by antisense mechanisms. In yet other embodiments, the inhibitory sequence or sequences are expressed as a hairpin RNA, which comprises both a sense sequence and an antisense sequence. In embodiments comprising a hairpin structure, the loop structure may comprise any suitable nucleotide sequence including for example 5′ untranslated regions of the gene to be suppressed, such as the 5′ UTR of AGP2 and the like (see for example, SEQ ID NO:16). In some embodiments, the AGP inhibitory sequence or sequences expressed as a hairpin are encoded by an inverted region of the AGP1 nucleotide sequence, the AGP2 nucleotide sequence, or a combination thereof. In yet other embodiments, the AGP inhibitory sequences are expressed as double-stranded RNA where one AGP1 inhibitory sequence, AGP2 inhibitory sequence, or a combination thereof is expressed in the sense orientation and another complementary sequence is expressed in the antisense orientation (see for example SEQ ID NO:16 and SEQ ID NO:18). Double-stranded RNA, hairpin structures, and combinations thereof comprising AGP1 nucleotide sequences, AGP2 nucleotide sequences, or combinations thereof may operate by RNA interference, cosuppression, antisense mechanism, any combination thereof, or by means of any other mechanism that causes inhibition of AGP expression or function. A more detailed description of each of these methods is described elsewhere herein below.

In one such embodiment, a nucleotide construct is produced, wherein the construct comprises a seed-preferred promoter (e.g., napin, phaseolin, β-conglycinin, oleosin, ltp (lipid transfer protein), EAP (embryo abundant protein), and the like), operably linked to a nucleotide sequence encoding either one of the subunits of AGP (i.e., AGP1 or AGP2). It is recognized that cereal seed germ includes the embryo proper and the scutellum, but as a whole is rich in oil compared to other materials stored in the organ. The nucleotide construct is built using the AGP1 or AGP2 coding sequence or portion thereof either in sense orientation, antisense orientation, and/or both a sense sequence and its complement of the AGP1 or AGP2 nucleotide sequence. Transformation of this nucleotide construct and an appropriate selectable marker into the proper crop plant, such as, corn, sunflower, soybean, cottonseed, or canola, ensure selection of plants with the ability to produce seed in which the AGP activity is inhibited. The result is reduced starch biosynthesis and an attendant increase in the concentration of oil in the seed. Where the plant has also been genetically manipulated to comprise a polynucleotide that provides for increased expression of the LEC1 transcriptional activator or functional variant or fragment thereof, the concentration of oil in the seed can be increased to unexpectedly high levels as demonstrated in the Experimental section herein below.

In some embodiments, the methods of the invention comprise introducing into the plant of interest at least one other polynucleotide of interest in addition to the first and second polynucleotides, wherein the additional polynucleotide(s), or expression of the additional polynucleotide(s), provide(s) for one or more additional phenotypic alterations in the plant. Virtually any third gene sequence of interest may be used to impart a phenotype of interest. In one such embodiment, the additional polynucleotide(s), or expression of the additional polynucleotide sequence(s), provide(s) for alteration of the quality of the increased oil produced within the plant by altering expression or function of proteins or enzymes that are involved in lipid modification. Examples of proteins and enzymes that modify the characteristics of oil within a plant include, but are not limited to, any of the fatty acid desaturases, for example, stearoyl-acyl-carrier-protein desaturase (Fad1; see U.S. Pat. No. 6,117,677), delta-15 desaturase (omega-3) (Fad3; Shah et al. (1997) Plant Physiol. 114: 1533-1539), delta-4 (trans) desaturase (Fad4; Xiao et al. (2001) J. Biol. Chem. 276: 31561-31566), delta-7 desaturase, (Fad5; see U.S. Pat. No. 6,635,451), omega-6 fatty-acid desaturase (Fad6; see U.S. Pat. No. 6,635,451), omega-3 fatty-acid desaturase (Fad7; Iba et al. (1993) J. Biol. Chem. 268: 24099-24105), delta-5 desaturase (see U.S. Pat. No. 6,589,767), delta-9-desaturase (see U.S. Pat. No. 5,723,595), fatty acyl-CoA:fatty alcohol acyltransferase (wax synthase; see U.S. Pat. No. 6,492,509), beta-ketoacyl-ACP synthase in an antisense or sense orientation (see U.S. Pat. No. 6,483,008), and delta-12 fatty acid desaturase (FAD2), an enzyme that converts oleic acid to linoleic acid by introducing a double bond at the delta-12 position (Okuley et al. (1994) Plant Cell 6: 147-58). FAD2 from a variety of plants is capable of catalyzing the formation of hydroxyl-, epoxy-, triple bond- and conjugated double bond-containing fatty acids—unusual fatty acids of commercial interest. In some embodiments, the additional polynucleotide encodes an inhibitory nucleotide molecule designed to silence expression of an enzyme involved in fatty acid desaturation, for example, an FAD such as FAD2.

Thus, in some embodiments, a plant into which a polynucleotide encoding a transcriptional activator such as LEC1 or functional fragment or variant thereof operably linked to a promoter functional in a plant cell and a polynucleotide comprising an AGP inhibitory sequence have been introduced is also provided with a third polynucleotide comprising an inhibitory sequence for an FAD, for example, an FAD2 inhibitory sequence, wherein the third polynucleotide is capable of inhibiting the expression or function of the targeted FAD. Plants expressing inhibitors of FAD or having the FAD gene deleted or inactivated have increased levels of specific oils such as increased levels of oleic acid and decreased levels of linoleic acid. In some embodiments, the additional polynucleotide to be introduced into the plant encodes an inhibitory nucleotide molecule designed to silence expression of an FAD such as FAD2, where the inhibitory nucleotide molecule is selected from the group consisting of sense-orientation RNA, antisense RNA, double-stranded RNA (dsRNA), hairpin RNA (hpRNA), intron-containing hpRNA, catalytic RNA, miRNA, and the like.

Examples of FAD2 and corresponding coding sequences are well known in the art. See for example GenBank Accession No. NM_{—}112047; GenBank Accession No. AF243045; European Patent No. EP0668919 B 1; U.S. Pat. No. 6,291,742; U.S. Pat. No. 6,310,194; U.S. Pat. No. 6,323,392; U.S. Pat. No. 6,372,965; U.S. Patent Application Publication No. 20030033633; and U.S. Patent Application Publication No. 20030140372; all of which are incorporated in their entirety herein by reference. In corn, two FAD2 proteins have been identified to date. One FAD2 protein is termed zmFAD2-1 (nucleotide sequence set forth in SEQ ID NO:12, amino acid sequence set forth in SEQ ID NO:13) and the other is termed zmFAD2-2 (Kinney et al. (2001) Biochem. Soc. Trans. 30: 1099-1103; and Mikkilineni et al. (2003) Theor. Appl. Genet. 106: 1326-1332). Suppression or deletion of both genes increases relative oleic acid concentration by 75%. Thus, by interfering with fatty acid desaturase activity, the saturation sites and the amount of saturation of an oil can be modified, for example, increasing or decreasing the amount of total omega or delta trans-fatty acids.

FAD2 converts the delta-12 single bond of oleic acid (C 18:1) into a conjugated double bond, thus producing linoleic acid (C18:2). Therefore, inhibiting the expression or function of FAD2 prevents the conversion of oleic acid into linoleic acid, and thus, oleic acid accumulates in the plant or plant part thereof such as seed, including seed embryos. “FAD2” is intended to mean any delta-12 fatty acid desaturase, including, but not limited to, the FAD2 proteins disclosed herein, such as zmFAD2-1 (SEQ ID NO:13), the polypeptide encoded by SEQ ID NO:12, zmFAD2-2, and the like. Increasing the production of oleic acid in grain is useful because oleic acid is more heat stable and may be used in applications such as frying, without having to first hydrogenate the oil using industrial processes.

Using the methods and compositions disclosed herein, total oil production can be increased and further more, the characteristics of the increased oil can be modified. Thus, in some embodiments of the invention, FAD2 expression or function is inhibited in combination with inhibition of AGP expression or function (i.e., targeting AGP1, AGP2, or combination thereof), or increasing expression of LEC1 (or functional fragment or variant thereof), or combinations thereof, and the oleic acid levels in embryos are thereby increased. In other embodiments of the invention, the levels of linoleic acid are decreased. In yet other embodiments, the levels of oleic acid are increased and the levels of linoleic acid are decreased in the same embryo.

In some embodiments, a sequence expressing LEC1 or functional fragment or variant thereof as noted herein below is coexpressed (either on the same or separate nucleotide constructs, located on the same or different vectors) with at least a portion of a nucleotide sequence encoding FAD2 operably linked to a promoter that is functional in a plant cell, thereby inhibiting the desaturase activity in the plant cell by any method known in the art or described herein. In some other embodiments, a LEC1 sequence is expressed in conjunction with an AGP1 inhibitory sequence, an AGP2 inhibitory sequence, or combinations thereof, and further in conjunction with an FAD2 inhibitory sequence (see, for example SEQ ID NO:18), wherein these sequences can be expressed as part of a single nucleotide construct or as part of two or even three separate nucleotide constructs, located on the same or different expression vectors.

Any nucleotide sequence encoding a desaturase known in the art can be used in the methods of the invention including, but not limited to, the desaturase nucleotide sequences described above. In one embodiment an FAD2 nucleotide sequence is expressed, such as zmFAD2-1 (SEQ ID NO:12), a nucleotide sequence encoding ZmFAD2-1 of SEQ ID NO:13, a zmFAD2-2 nucleotide sequence, combinations of both, and the like. See, for example, the sequences disclosed in Mikkilineni et al. (2003) Theor. Appl. Genet. 106: 1326-1332. In some embodiments, the FAD2 sequence is selected from, but not limited to, those disclosed in GenBank Accession No. NM_{—}112047, GenBank Accession No. AF243045, U.S. Pat. No. 6,323,392, U.S. Pat. No. 6,372,965, U.S. Patent Application Publication No. 20030033633, and U.S. Patent Application Publication No. 20030140372, all of which are incorporated herein in their entirety by reference. In other embodiments, the FAD2 inhibitory sequence comprises truncated regions of the ZmFAD2 nucleotide sequence, for example, ZmFAD2-1 of SEQ ID NO:12, in the sense orientation and antisense orientation; see, for example, SEQ ID NO:17.

The methods of the invention can additionally involve any method or mechanism known in the art for reducing or eliminating the expression or function of FAD2 in a cell including, but not limited to, antisense suppression, sense suppression, cosuppression-inducing polynucleotide fusion chimeras of AGP and another gene of interest, RNA interference, directed deletion or mutation, and dominant-negative strategies. Thus, the methods and compositions disclosed herein are not limited to any mechanism or theory of action and include any method where fatty acid desaturase activity is inhibited.

For example, in some embodiments the FAD to be inhibited is FAD2, and the FAD2 inhibitory sequence is expressed in the sense orientation. In some embodiments, the sense-oriented transcripts cause cosuppression. In other embodiments, the sense-orientated expression of truncated FAD2 transcripts cause nonfunctional proteins to be expressed and thus inhibit FAD2 activity. Alternatively, the inhibitory FAD2 sequence or sequences can be expressed in the antisense orientation and thus inhibit endogenous FAD2 expression or activity by antisense mechanisms. In yet other embodiments, the FAD2 inhibitory sequence or sequences are expressed as a hairpin RNA, which has both a sense sequence and an antisense sequence component. In embodiments comprising a hairpin structure, the loop structure may comprise any suitable nucleotide sequence including for example 5′ untranslated regions of the gene to be suppressed, such as the 5′ UTR of FAD2, intron nucleotide sequences of alcohol dehydrogenase (adh1), random nucleotides, polynucleotide spacers, and the like (see, for example, SEQ ID NO:18; see also Bailey-Serres and Dawe (1996) Plant Physiol. 112: 685). In some embodiments, the FAD2 inhibitory sequence or sequences expressed as a hairpin are encoded by an inverted region of FAD2-encoding nucleotide sequences such as the zmFAD2-1 nucleotide sequence (SEQ ID NO:12), the zmFAD2-2 nucleotide sequence, or a combination thereof. In yet other embodiments, the inhibitory sequences are expressed as double-stranded RNA where one inhibitory FAD2 sequence is expressed in the sense orientation and another complementary sequence is expressed in the antisense orientation (see, for example, SEQ ID NO:17 and SEQ ID NO:18). Double-stranded RNA, hairpin structures, and combinations thereof comprising FAD2 sequences may operate by RNA interference, cosuppression, antisense mechanism, any combination thereof, or by means of any other mechanism that causes inhibition of FAD2 expression or function.

Those skilled in the art recognize that the embodiments disclosed herein are not limited to the type or kind of promoter used to drive expression of the operably linked nucleotide sequences in the manner set forth herein as long as expression or function of the gene product of interest (i.e., the target gene product) is either increased (i.e., in the case of transcriptional activators such as LEC1, whereby lipid biosynthesis is increased) or inhibited (i.e., in the case of gene products that are involved in starch biosynthesis and/or storage, for example, AGP, whereby inhibition results in disruption of starch biosynthesis and/or storage are disrupted; or gene products that are involved in lipid desaturation, for example, a FAD such as FAD2, whereby inhibition results in disruption of lipid desaturase activity) in the plant of interest or a plant part thereof. Therefore any promoter that is functional in a plant, including, but not limited to the promoters disclosed herein, can be used to practice the methods of the present invention, and choice of promoter will depend upon the desired alteration in oil phenotype.

Thus, for example, in some embodiments, the FAD2 inhibitory sequence is operably linked to a promoter and assembled within a nucleotide construct that is independent of the nucleotide construct or nucleotide constructs comprising the other nucleotide sequences of interest, such as LEC1-encoding sequences and AGP inhibitory sequences, where the other nucleotide sequences of interest are operably linked to the same or different promoters from the promoter driving expression of the FAD2 inhibitory sequence. In other embodiments, the FAD2 inhibitory sequence can be expressed on the same construct as the other nucleotide sequences of interest, where the FAD2 inhibitory sequence and other nucleotide sequences of interest are operably linked to the same promoter or different promoters.

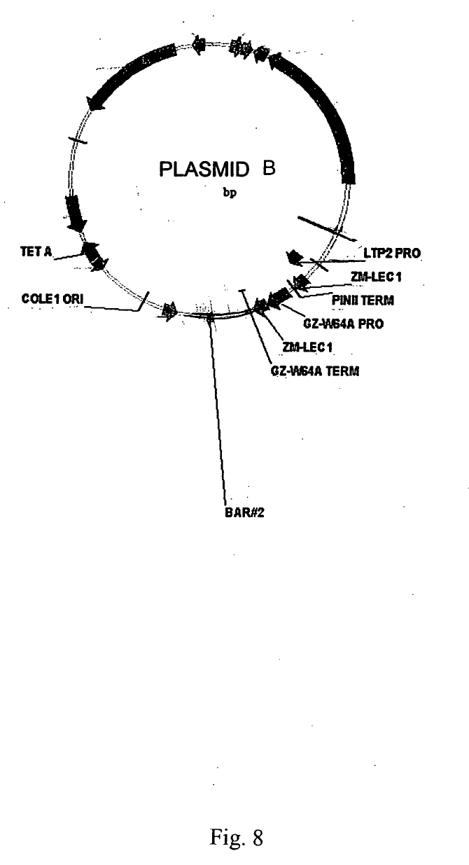

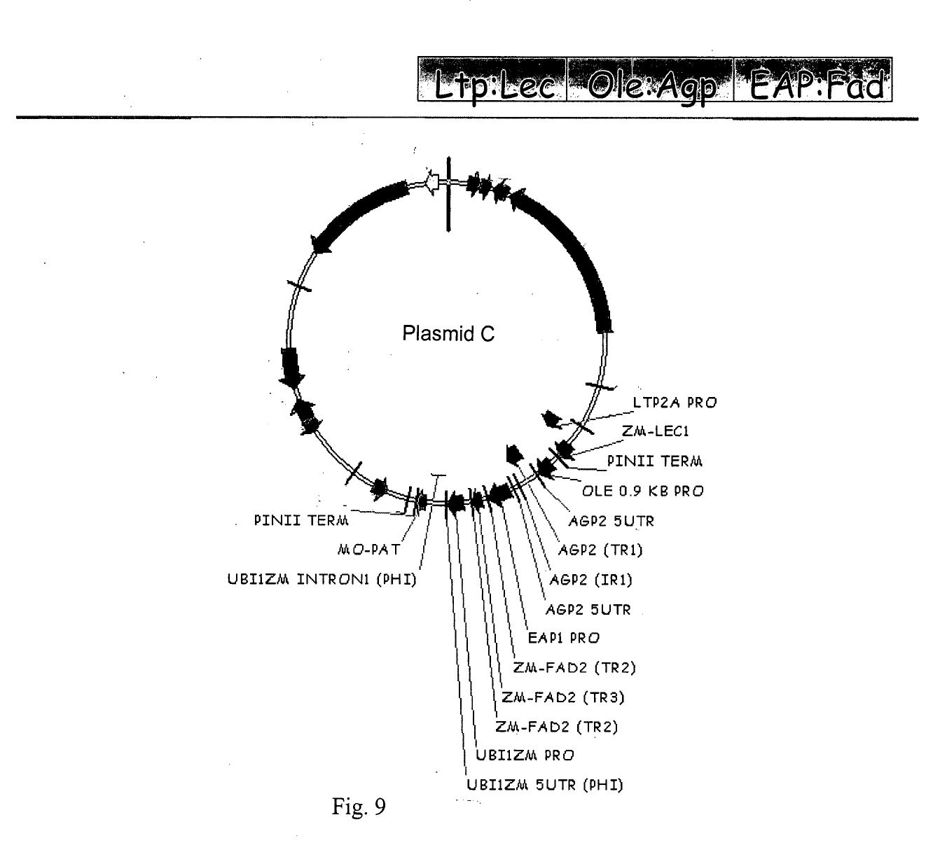

In some embodiments, LEC1 expression is driven by an LTP2 (lipid transfer protein-2) promoter. In other embodiments, LEC1 expression is driven by a gamma zein promoter. In yet other embodiments where multiple copies of the LEC1 coding sequence are present on the same plasmid, a gamma zein promoter drives expression in one or more cassettes while the LTP2 promoter drives expression in one or more cassettes. See, for example, FIG. 8. Accordingly, expression of an AGP inhibitory sequence or sequences can be driven by any suitable promoter, whether the AGP inhibitory sequence is on the same or a different vector as LEC1. In some embodiments, AGP inhibitory sequences (e.g., AGP1 inhibitory sequences, AGP2 inhibitory sequences, or combinations thereof) can be driven by a seed-preferred promoter such as the oleosin promoter, the EAP (embryo abundant protein) promoter, and the like. In yet other embodiments, a fusion construct comprising at least one AGP1 inhibitory sequence and at least one AGP2 inhibitory sequence (see for example, SEQ ID NO:15) is driven under a seed-preferred promoter such as the oleosin promoter, the EAP promoter, and the like, and LEC1 (for example, SEQ ID NO:1) expression is driven under the same or different promoter, for example, the LTP2 promoter (see, for example, FIG. 6).

Where LEC1 expression is to be increased in combination with inhibition of AGP activity and FAD activity, for example, FAD2 activity, any combination of suitable promoters can be used to drive expression of the operably linked polynucleotides of interest. Thus, for example, a seed-preferred promoter such as the oleosin promoter, the EAP, and the like can drive expression of the operably linked FAD2 inhibitory sequence. In other embodiments, the same promoter that drives expression of an operably linked FAD2 inhibitory sequence may be used to drive expression of other nucleotide sequences of interest such as an AGP1 inhibitory sequence, an AGP2 inhibitory sequence, or a combination thereof, and a LEC1-encoding sequence. For example, a seed-preferred promoter such as oleosin, EAP, and the like may drive expression of one or both of an FAD2 inhibitory sequence and an AGP inhibitory sequence (whether on the same or different constructs or vectors) such as an AGP1 inhibitory sequence, an AGP2 inhibitory sequence, or a combination thereof, and LEC1 expression can be driven by the same or a different promoter, for example, the LTP2 promoter. See, for example, FIG. 9.

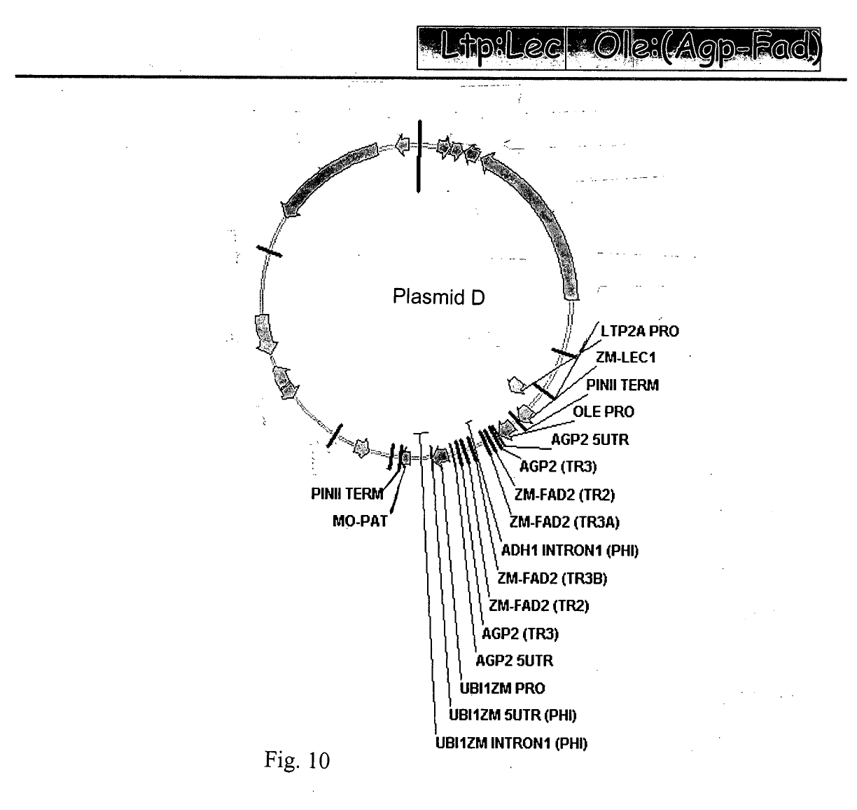

In yet other embodiments, a fusion construct comprising at least one FAD2 inhibitory sequence and at least one AGP inhibitory sequence (such as an AGP1 inhibitory sequence, an AGP2 inhibitory sequence, or combination thereof) is operably linked to a seed-preferred promoter such as the EAP (embryo abundant protein) promoter, the oleosin promoter, and the like, and the same or a different promoter, for example, LTP2, is used to drive expression of LEC1. See, for example, FIG. 10.

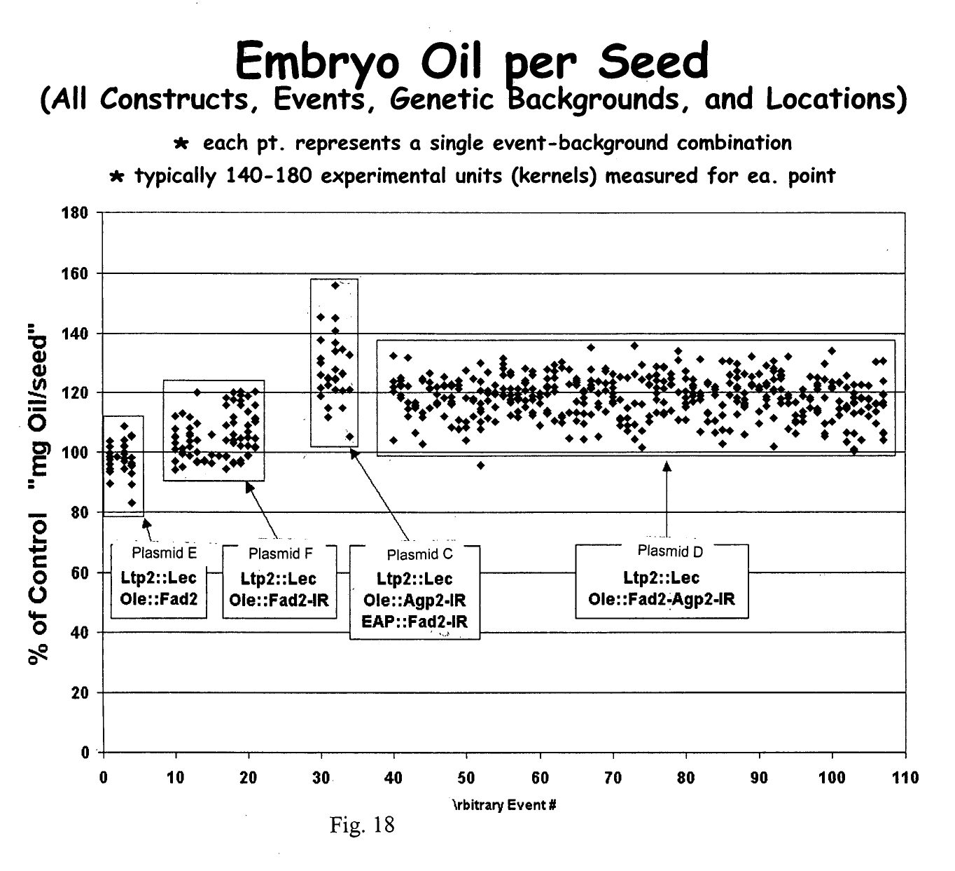

It has been unexpectedly found that the method of inhibition of AGP and FAD (e.g., whether by means of a fusion construct or independently expressed inhibitory sequences) may confer additional phenotypes in the transformant. For example, use of a fusion construct of an AGP inhibitory polynucleotide sequence and an FAD2 inhibitory polynucleotide sequence results in less variability and increased consistency of oil production among transformants as shown in FIG. 18. Reciprocally, use of independently transcribed AGP inhibitory sequences and FAD2 inhibitory sequences results in greater variability among transformants, but, in certain instances, a concomitant increase in total oil production. See, for example, FIG. 18. Thus, the choice of constructs will depend on the desired phenotype to be conferred to the transformant.

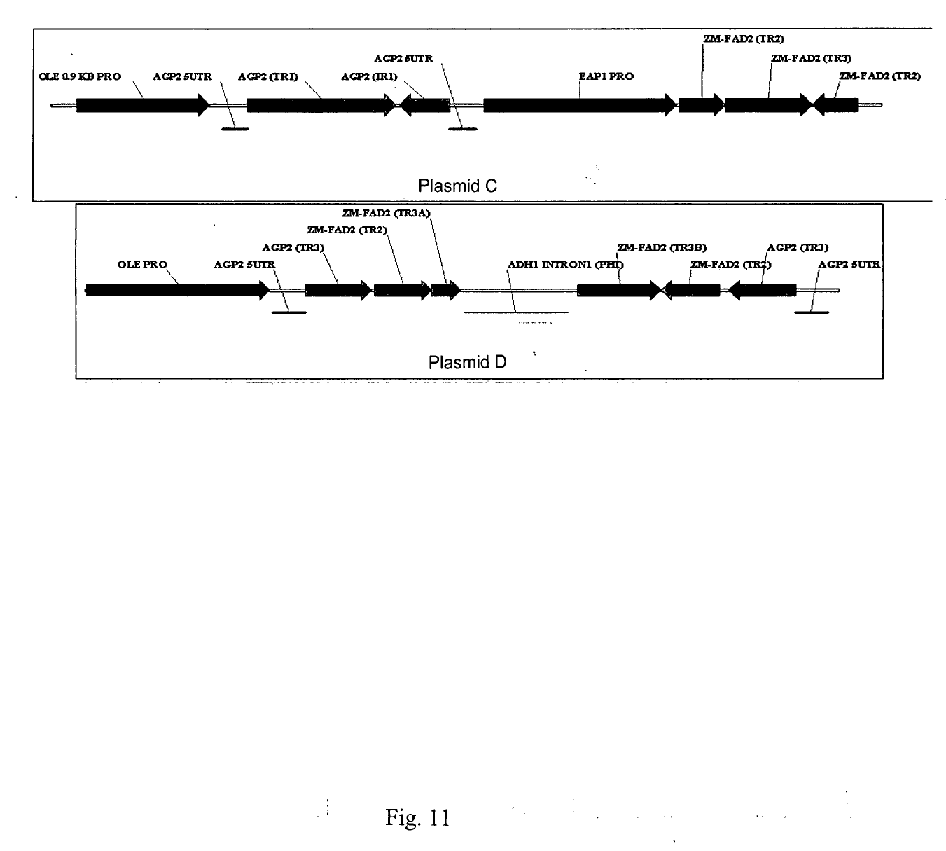

In certain embodiments, an FAD2 inhibitory sequence and an AGP2 inhibitory sequence are expressed as a fusion construct wherein the RNA transcription product is capable of inhibiting both AGP and FAD2. Nucleotide constructs comprising such a fusion construct are disclosed in FIGS. 10 and 11; see also the AGP2-FAD2 fusion construct shown in SEQ ID NO:18. In some other embodiments, a fusion construct capable of expressing a fusion polynucleotide that can simultaneously inhibit AGP and FAD, for example FAD2, may be used to improve consistency of downregulation of AGP enzyme activity and/or FAD enzyme activity among transformants, for example in systems where oleic acid/linoleic acid content is to be regulated in a plant. Such AGP-FAD inhibitory fusion constructs of the invention may be used alone or in conjunction with other methods and constructs to modify oil phenotype. Thus, in some embodiments the AGP-FAD inhibitory fusion constructs of the invention are co-transformed with other constructs of interest. Such constructs include but are not limited to those capable of expressing LEC1, any other polynucleotide, or polypeptide, or a combination thereof. (See, for example, Example 6, infra). AGP-FAD inhibitory fusion constructs comprise those capable of expressing polynucleotides such as siRNA, hpRNA, ihpRNA, antisense RNA, combinations thereof, and the like.

In yet other embodiments, an FAD inhibitory sequence, for example an FAD2 inhibitory sequence and an AGP inhibitory sequence are expressed independently under separate promoters. Thus, the co-expressed transcription products wherein one inhibits AGP activity (see, for example, SEQ ID NO:16) and another inhibits FAD activity (see, for example SEQ ID NO:17) can vary oil production and characteristics in a cell as shown, for example, in FIG. 18. Such co-expressed AGP inhibitory constructs and FAD inhibitory constructs of the invention may be used alone or in conjunction with other methods and constructs to modify oil phenotype. Thus, in some embodiments the co-expressed AGP inhibitory constructs and FAD inhibitory constructs of the invention are co-transformed with other constructs of interest. Such constructs include but are not limited to those capable of expressing LEC1, any other polynucleotide, or polypeptide, or a combination thereof. Co-expressed AGP inhibitory constructs and FAD inhibitory constructs of the invention comprise those capable of expressing polynucleotides such as siRNA, hpRNA, ihpRNA, antisense RNA, combinations thereof, and the like.

Those skilled in the art recognize that any nucleotide sequence of interest can be transcribed in conjunction with a LEC1-encoding sequence and AGP inhibitory sequence. Therefore, the use of FAD inhibitory sequences, for example, FAD2 inhibitory sequences, to confer additional phenotypes to a plant comprising one or more nucleotide constructs that provide for the expression of LEC1 or other transcriptional activator capable of increasing lipid biosynthesis in combination with expression of AGP inhibitory sequences or other inhibitory sequences that are capable of inhibiting starch biosynthesis and/or starch storage in any of the embodiments described herein is offered by way of example and not by way of limitation.

It is recognized that in using the methods of the present invention, the combining of desired traits in plants can be subtractive, additive, or synergistic. Subtractive effects occur where a second event (e.g., transformation with a second gene or crossbreeding with a second plant expressing a gene of interest) decreases the oil production in a plant relative to the first event (e.g., transformation with a first gene or crossbreeding with a first plant expressing a gene of interest). For example, where two traits individually increase oil production in a plant by 10% relative to oil production in a wild-type plant, combining the traits leads to a decrease in oil production relative to oil production in a wild-type plant. Additive effects occur where a second event (e.g., transformation with a second gene or crossbreeding with a second plant expressing a gene of interest) increases the oil production in a plant relative to the first event (e.g., transformation with a first gene or crossbreeding with a first plant expressing a gene of interest), but only to the extent that each independent event increased oil production. For example, where each trait increases oil production in a plant by 10%, the combination increases oil production in a plant up to, but not exceeding, 20% relative to oil production in a wild-type plant. Synergistic effects occur where the increase exceeds that of the increase observed when the independent effects of the two events are additive. For example, where each independent transformation event results in a 10% increase in oil production in a plant, a combination of the two events results in greater than a 20% increase in oil production in that plant relative to oil production in a wild-type plant. “Increase in oil production” is defined herein as either additive or synergistic increases in oil production.

In accordance with the methods of the present invention, these novel combinations of polynucleotide sequences are introduced into a plant of interest in order to manipulate oil and starch metabolism in favor of increased oil production and/or modification of the characteristics of the oil being produced. The compositions and methods of the invention provide for control of both lipid and starch biosynthesis, which has a dramatic effect on oil production. Any sequence encoding a transcriptional activator of interest, for example, a LEC1 polypeptide can be used in combination with AGP inhibitory sequencers), and optionally FAD inhibitory sequences, for example, FAD2 inhibitory sequences, to practice the methods of the present invention. The nucleotide sequences can be based on the native (i.e., naturally occurring) coding sequences, for example, the native LEC1 coding sequence shown in SEQ ID NO:1. Alternatively, fragments and variants of the disclosed polynucleotides and proteins encoded thereby can be used to practice the present invention.

“Fragment” is intended to mean a portion of the polynucleotide or a portion of the amino acid sequence and hence protein encoded thereby. Fragments of a polynucleotide may encode protein fragments that retain the biological activity of the native protein, for example, LEC1 activity where the fragment is a portion of a LEC1 transcriptional activator, such as the LEC1 transcriptional activator of SEQ ID NO:2. Such protein fragments are referred to herein as “functional” protein fragments. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the proteins contemplated in the methods and compositions of the invention.

For example, a fragment of a LEC1 polynucleotide that encodes a biologically active portion of a LEC1 protein will encode at least 15, 25, 30, 50, 100, 150, 200, or 250 contiguous amino acids, or up to the total number of amino acids present in a full-length LEC1 protein, for example, 278 amino acids for SEQ ID NO:2. Fragments of a LEC1 polynucleotide that are useful as hybridization probes or PCR primers generally need not encode a biologically active portion of a LEC1 protein.

Thus, a fragment of a LEC1 polynucleotide may encode a biologically active portion of a LEC1 protein, or it may be a fragment that can be used as a hybridization probe or PCR primer using methods disclosed below to identify other LEC1-like polynucleotides for use in the compositions and methods of the present invention. A biologically active portion of a LEC1 protein can be prepared by isolating a portion of a LEC1 polynucleotide, for example, the LEC1 polynucleotide set forth in SEQ ID NO:1, expressing the encoded portion of a LEC1 protein (e.g., by recombinant expression in vitro), and assessing the activity of the encoded portion of the LEC1 protein, particularly the ability to enhance lipid synthesis as reflected in increased oil production. Polynucleotides that are fragments of a LEC1 nucleotide sequence comprise at least 16, 20, 50, 75, 100, 150, 200, 250, 300, 350, 400, 450, 500, 550, 600, 650, 700, 800 nucleotides, or up to the number of nucleotides present in a full-length LEC1 polynucleotide disclosed herein (for example, 837 nucleotides for SEQ ID NO:1).

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of a polypeptide of interest, for example, one of the LEC1 polypeptides described herein. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide of interest, for example, a LEC1 protein described herein, such as the LEC1 protein set forth in SEQ ID NO:2. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO:2 are disclosed and can be used to practice the present invention. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant LEC1 proteins encompassed by the present invention are biologically active, that is they continue to possess the desired biological activity of the native protein, that is, LEC1 activity as described herein, particularly the ability to enhance lipid synthesis as reflected in increased oil production. Such variants may result from, for example, genetic polymorphism or from human manipulation. Biologically active variants of a native LEC1 protein, for example, the LEC1 protein set forth in SEQ ID NO:2, will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of interest, for example, the LEC1 protein of SEQ ID NO:2, may differ from that protein by as few as 1-15 amino acid residues, as few as 1-10, such as 6-10, as few as 5, as few as 4, 3, 2, or even 1 amino acid residue.

Variant LEC1 polypeptides (such as the Hap3/Lec1 CCAAT family of transcriptional proteins), as well as polynucleotides encoding these variants, are known in the art. See, for example, U.S. Patent Application Publication No. 20030126638, Table 1 and FIG. 1, and GenBank Accession No. AF410176 (Zea mays LEC1). For example, within the “B domain” of all plant LEC1 sequences examined, a highly conserved CCAAT-box binding motif has been found to contain the non-variable residues methionine, proline, isoleucine, alanine, asparagine, valine, and isoleucine (MPIANVI), see U.S. Pat. No. 6,825,397. Functional fragments and biologically active variants of a LEC1 polypeptide, for example, LEC1 of SEQ ID NO:2 comprise a conserved region of the consensus sequence set forth herein as SEQ ID NO:14.

The proteins for use in practicing the present invention may be altered in various ways including amino acid substitutions, deletions, truncations, and insertions. Methods for such manipulations are generally known in the art. For example, amino acid sequence variants and fragments of the LEC1 proteins can be prepared by mutations in the DNA. Methods for mutagenesis and polynucleotide alterations are well known in the art. See, for example, Kunkel (1985) Proc. Natl. Acad. Sci. USA 82: 488-492; Kunkel et al. (1987) Methods in Enzymol. 154: 367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides that can be used to practice present invention include both the naturally occurring sequences as well as mutant forms. Likewise, the proteins for use in practicing the present invention encompass both naturally occurring proteins as well as variations and modified forms thereof. Such variants will continue to possess the desired biological activity, for example, the desired LEC1 activity of enhancing lipid synthesis. Obviously, the mutations that will be made in the DNA encoding the variant must not place the sequence out of reading frame and optimally will not create complementary regions that could produce secondary mRNA structure. See, EP Patent Application Publication No. 75, 444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays, such as the effect of changes to the protein on embryogenesis and oil content. See, for example U.S. Patent Application No. 20030167526 and U.S. Pat. No. 6,232,529 B1, herein incorporated by reference.

Variant polynucleotides and proteins also encompass sequences and proteins derived from a mutagenic and recombinogenic procedure such as DNA shuffling. With such a procedure, one or more different LEC1 coding sequences can be manipulated to create a new LEC1 polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a LEC1 sequence disclosed herein (for example, the sequence of SEQ ID NO:1) and other known genes in the Lec transcription family to obtain a new gene coding for a LEC protein with an improved property of interest, such as an increased ability to enhance lipid synthesis. Strategies for such DNA shuffling are known in the art. See, for example, Stemmer (1994) Proc. Natl. Acad. Sci. USA 91: 10747-10751; Stemmer (1994) Nature 370: 389-391; Crameri et al. (1997) Nature Biotech. 15: 436-438; Moore et al. (1997) J. Mol. Biol. 272: 336-347; Zhang et al. (1997) Proc. Natl. Acad. Sci. USA 94: 4504-4509; Crameri et al. (1998) Nature 391: 288-291; and U.S. Pat. Nos. 5,605,793 and 5,837,458.

A method for modulating the concentration and/or activity of a target polypeptide of interest in a plant as a result of practicing the present invention is provided. In general, concentration and/or activity is increased or decreased by at least 1%, 5%, 10%, 20%, 30%, 40%, 50%, 60%, 70%, 80%, or 90% relative to a native control plant, plant part, or plant cell that did not have the combination of polynucleotides of the present invention introduced. Modulation in the present invention may occur during and/or subsequent to growth of the plant to the desired stage of development. In specific embodiments, LEC1, AGP (i.e., AGP1, AGP2, or combination thereof), and optionally FAD such as FAD2, are modulated in monocots, particularly maize.

The expression level of LEC1, AGP, and FAD such as FAD2, and oil content may be measured directly, for example, by assaying the level of LEC1, AGP, FAD such as FAD2, or oil in a plant or indirectly by measuring the transcriptional activity of LEC1 (see for example, Lee et al. (2003) Proc. Natl. Acad. Sci. USA 100: 2152-2156); the ADP-glucose pyrophosphorylase (AGPase) activity of AGP (Singletary et al. (1990) Plant Physiol. 94: 858-864), the fatty acid desaturase activity of FAD such as FAD2 (see for example Miquel et al. (1992) J. Biol. Chem. 267: 1502-1509); see also, U.S. Patent Application Publication No. 20030167526 and U.S. Pat. No. 6,232,529 B1 as discussed above; all of which are herein incorporated by reference in their entirety. In some embodiments, the chemical structure of the oil is also measured as well as the absolute concentration of all oil. Assays to measure the concentration of specific oils are routine in the art. For example, the level of oleic acid and linoleic acid can be measured using chromatographic techniques such as HPLC and gas chromatography, as well as physical techniques such as near-infrared spectral analysis (see for example, Moon et al. (2000) Lipids 35: 471-479).

The following terms are used to describe the sequence relationships between two or more polynucleotides or polypeptides: (a) “reference sequence,” (b) “comparison window,” (c) “sequence identity,” and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4: 11-17; the local alignment algorithm of Smith et al. (1981) Adv. Appl. Math. 2: 482; the global alignment algorithm of Needleman and Wunsch (1970) J. Mol. Biol. 48: 443-453; the search-for-local alignment method of Pearson and Lipman (1988) Proc. Natl. Acad. Sci. 85: 2444-2448; the algorithm of Karlin and Altschul (1990) Proc. Natl. Acad. Sci. USA 872264, modified as in Karlin and Altschul (1993) Proc. Nail. Acad. Sci. USA 90: 5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73: 237-244 (1988); Higgins et al. (1989) CABIOS 5: 151-153; Corpet et al. (1988) Nucleic Acids Res. 16: 10881-90; Huang et al. (1992) CABIOS 8: 155-65; and Pearson et al. (1994) Meth. Mol. Biol. 24: 307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215: 403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” is intended to mean any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89: 10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotides comprising LEC1-encoding sequences, the AGP inhibitory sequence(s), the FAD inhibitory sequence(s), and combinations thereof can be provided in expression cassettes for transcription in the plant, plant cell, plant tissue (for example, embryo), or plant part (for example, seed) of interest. The cassette will include 5′ and 3′ regulatory sequences operably linked to a LEC1-encoding polynucleotide (for example, the sequence set forth in SEQ ID NO:1 or variant or fragment thereof), an AGP inhibitory sequence (for example, an AGP1 inhibitory sequence, an AGP2 inhibitory sequence, or combination thereof), an FAD inhibitory sequence (for example, an FAD2 inhibitory sequence), and any combinations thereof “Operably linked” is intended to mean a functional linkage between two or more elements. For example, an operable linkage between a polynucleotide of interest and a regulatory sequence (i.e., a promoter) is a functional link that allows for transcription of the polynucleotide of interest. Nucleotide sequences forming a fusion construct, for example, two inhibitory polynucleotide sequences that are to be transcribed by a single operably linked promoter, are also considered to be operably linked to each other. Operably linked elements may be contiguous or non-contiguous. When used to refer to the joining of two protein-encoding regions that are translated as proteins, by “operably linked” is intended that the coding regions are in the same reading frame. The cassette may additionally contain at least one additional gene to be cotransformed into the organism. Alternatively, the additional gene(s) can be provided on multiple cassettes. Such a cassette is provided with a plurality of restriction sites and/or recombination sites for insertion of a LEC1-encoding polynucleotide, an AGP inhibitory sequence, an FAD inhibitory sequence, and combinations thereof to be under the transcriptional regulation of the regulatory regions. The cassette may additionally contain selectable marker genes.

The expression cassette will include in the 5′-3′ direction of transcription, a transcriptional and translational initiation region (i.e., a promoter), a LEC1-encoding polynucleotide, an AGP inhibitory sequence, an FAD inhibitory sequence, and combinations thereof in accordance with the present invention, and a transcriptional and translational termination region (i.e., termination region) functional in plants. The regulatory regions (i.e., promoters, transcriptional regulatory regions, and translational termination regions) and/or a LEC1-encoding polynucleotide, an AGP inhibitory sequence, an FAD inhibitory sequence, and combinations thereof may be native/analogous to the host cell or to each other. Alternatively, the regulatory regions and/or the LEC1-encoding polynucleotide, the AGP inhibitory sequence, the FAD inhibitory sequence, and combinations thereof may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of LEC1, AGP inhibitory sequences, FAD inhibitory sequences, and combinations thereof in the plant, plant cell, plant tissue, or plant part of interest. Thus, the phenotype of the plant, plant cell, plant tissue, or plant part of interest can be altered.

The termination region may be native with the transcriptional initiation region, may be native with the operably linked LEC1-encoding polynucleotide, the AGP inhibitory sequence, the FAD inhibitory sequence, and combinations thereof may be native with the plant host, or may be derived from another source (i.e., foreign or heterologous to the promoter, the LEC1-encoding polynucleotide, the AGP inhibitory sequence, the FAD inhibitory sequence, and combinations thereof). Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64: 671-674; Sanfacon et al (1991) Genes Dev. 5: 141-149; Mogen et al (1990) Plant Cell 2: 1261-1272; Munroe et al. (1990) Gene 91: 151-158; Ballas etal (1989) Nucleic Acids Res. 17: 7891-7903; and Joshi et al (1987) Nucleic Acids Res. 15: 9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17: 477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86: 6126-6130); potyvirus leaders, for example, TEV leader (Tobacco Etch Virus) (Gallie et al. (1995) Gene 165(2): 233-238), MDMV leader (Maize Dwarf Mosaic Virus), and human immunoglobulin heavy-chain binding protein (BiP) (Macejak et al. (1991) Nature 353: 90-94); untranslated leader from the coat protein mRNA of alfalfa mosaic virus (AMV RNA 4) (Jobling et al. (1987) Nature 325: 622-625); tobacco mosaic virus leader (TMV) (Gallie et al (1989) in Molecular Biology of RNA, ed. Cech (Liss, New York), pp. 237-256); and maize chlorotic mottle virus leader (MCMV) (Lommel et al. (1991) Virology 81: 382-385). See also, Della-Cioppa et al. (1987) Plant Physiol. 84: 965-968. In preparing a cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. Suitable promoters will be functional in a plant cell into which they are introduced. By “functional” is intended the promoter, when operably linked to a sequence encoding a protein of interest, is capable of driving expression (i.e., transcription and translation) of the encoded protein, or, when operably linked to an inhibitory sequence encoding an inhibitory nucleotide molecule (for example, a hairpin RNA, double-stranded RNA, miRNA polynucleotide, and the like), the promoter is capable of initiating transcription of the operably linked inhibitory sequence such that the inhibitory nucleotide molecule is expressed. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313: 810-812); rice actin (McElroy et al. (1990) Plant Cell 2: 163-171); ubiquitin (Christensen et al. (1989) Plant Mol. Biol. 12: 619-632 and Christensen et al. (1992) Plant Mol. Biol. 18: 675-689); pEMU (Last et al. (1991) Theor. Appl. Genet. 81: 581-588); MAS (Velten et al. (1984) EMBO J. 3: 2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142; and 6,177,611.

Tissue-preferred promoters can be utilized to target expression of LEC1, inhibitors of AGP, inhibitors of FAD, and combinations thereof, within a particular plant tissue. Tissue-preferred promoters include Yamamoto et al. (1997) Plant J. 12(2): 255-265; Kawamata et al. (1997) Plant Cell Physiol. 38(7): 792-803; Hansen et al. (1997) Mol. Gen Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Matsuoka et al. (1993) Proc Natl. Acad. Sci. USA 90(20): 9586-9590; and Guevara-Garcia et al. (1993) Plant J. 4(3): 495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10: 108, herein incorporated by reference. Such seed-preferred promoters include, but are not limited to, LTP2 (lipid transfer protein 2, which expresses in the aleurone; see Morino et al. (1999) Plant J. 17: 275-285 and U.S. Pat. No. 5,525,716, describing the barley LTP2 promoter, and International Publication No. WO 00/11177, describing the maize LTP2 promoter); Cim1 (cytokinin-induced message, which expresses in nucellus tissue; see U.S. Pat. No. 6,225,529, describing the maize Cim1 promoter); cZ19B1 (maize 19 kDa zein; see U.S. Pat. No. 6,225,529); milps (myo-inositol-1-phosphate synthase; see WO 00/11177 and U.S. Pat. No. 6,225,529; herein incorporated by reference). Gamma-zein is a representative endosperm-specific promoter. Globulin 1 (Glb-1; see GenBank Accession No. L22344), oleosin promoters (see, for example, Plant et al. (1994) Plant Mol. Biol. 25: 193-205; Keddie et al. (1994) Plant Mol. Biol. 24: 327-40; Keddie et al. (1992) Plant Mol. Biol. 19: 443-53; and Hong et al. (1997) 34: 549-55; see also, GenBank Accession Nos. U71381, AF134411, and U.S. Pat. No. 5,977,436, which contain oleosin promoter sequences from Glycine max, Brassica juncea, and Arabidopsis thaliana, respectively), and EAP 1 (early abundant protein 1; see U.S. Patent Application Publication No. 20040210043) are representative promoters that express in an embryo-preferred manner. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein (such as gzw64A promoter, see GenBank Accession No. S78780), waxy, shrunken 1, shrunken 2 (see, for example, Shaw et al. (1992) Plant. Physiol. 98: 1214-1216, and Zhong Chen et al. (2003) Proc. Natl. Acad. Sci. USA 100: 3525-3530), Globulin 1, oleosin, LTP2 (Kalla et al. (1994) Plant J. 6: 849-860; U.S. Pat. No. 5,525,716), nuc1 promoter (expressing in the nucellus tissue; see, for example, U.S. Pat. No. 6,407,315), Zm40 promoter (U.S. Pat. No. 6,403,862), mlip 15 promoter (U.S. Pat. No. 6,479,734), Zea mays L3 oleosin promoter (P-ZmL3; see, for example, Hong et al. (1997) Plant Mol. Biol. 34: 549-555), etc. See also WO 00/12733 and U.S. Pat. No. 6,528,704, where seed-preferred promoters from maize end1 and end2 genes are disclosed; each of which is herein incorporated by reference.

Although expression of nucleotide constructs of the invention may be directed to any tissue or combination of tissues, in certain embodiments inhibition of AGP, FAD2, or a combination thereof occurs in at least the embryo while expression of LEC1 occurs in at least the aleurone layer. Such targeted expression may be accomplished by any means described herein or known in the art. In some embodiments, the targeted inhibition of AGP, FAD2, or a combination thereof is accomplished by placing an inhibitory construct under the control of a promoter that drives expression of the construct in the embryo, including such promoters as the oleosin promoter. In some other embodiments, LEC1 expression is targeted to the aleurone layer by placing a polynucleotide capable of expressing LEC1 under regulatory control of a promoter that drives expression in at least the aleurone layer, such as, for example, the LTP2 promoter.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85: 610-9 and Fetter et al. (2004) Plant Cell 16: 215-28), cyan florescent protein (CYP) (Bolte et al. (2004) J. Cell Science 117: 943-54 and Kato et al. (2002) Plant Physiol 129: 913-42), and yellow florescent protein (PhiYFP™ from Evrogen, see, Bolte et al. (2004) J. Cell Science 117: 943-54). For additional selectable markers, see generally, Yarranton (1992) Curr. Opin. Biotech. 3: 506-511; Christopherson et al. (1992) Proc. Natl. Acad. Sci. USA 89: 6314-6318; Yao et al. (1992) Cell 71: 63-72; Reznikoff (1992) Mol. Microbiol. 6: 2419-2422; Barkley et al. (1980) in The Operon, pp. 177-220; Hu et al. (1987) Cell 48: 555-566; Brown et al (1987) Cell 49: 603-612; Figge et al. (1988) Cell 52: 713-722; Deuschle et al. (1989) Proc. Natl. Acad Aci. USA 86: 5400-5404; Fuerst et al. (1989) Proc. Natl. Acad. Sci. USA 86: 2549-2553; Deuschle et al. (1990) Science 248: 480-483; Gossen (1993) Ph.D. Thesis, University of Heidelberg; Reines et al. (1993) Proc. Natl. Acad. Sci. USA 90: 1917-1921; Labow et al. (1990) Mol. Cell. Biol. 10: 3343-3356; Zambretti et al. (1992) Proc. Natl. Acad. Sci. USA 89: 3952-3956; Baim et al. (1991) Proc. Natl. Acad. Sci. USA 88: 5072-5076; Wyborski et al. (1991) Nucleic Acids Res. 19: 4647-4653; Hillenand-Wissman (1989) Topics Mol. Struc. Biol. 10: 143-162; Degenkolb et al. (1991) Antimicrob. Agents Chemother. 35: 1591-1595; Kleinschnidt et al. (1988) Biochemistry 27: 1094-1104; Bonin (1993) Ph.D. Thesis, University of Heidelberg; Gossen et al. (1992) Proc. Natl. Acad. Sci. USA 89: 5547-5551; Oliva et al. (1992) Antimicrob. Agents Chemother. 36: 913-919; Hlavka et al. (1985) Handbook of Experimental Pharmacology, Vol. 78 (Springer-Verlag, Berlin); Gill et al. (1988) Nature 334: 721-724. Such disclosures are herein incorporated by reference.

The above list of selectable marker genes is not meant to be limiting. Any selectable marker gene can be used in the present invention.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4: 320-334), electroporation (Riggs et al. (1986) Proc. Natl. Acad. Sci. USA 83: 5602-5606, Agrobacterium-mediated transformation (U.S. Pat. No. 5,563,055 and U.S. Pat. No. 5,981,840), direct gene transfer (Paszkowski et al. (1984) EMBO J 3: 2717-2722), and ballistic particle acceleration (see, for example, U.S. Pat. Nos. 4,945,050; U.S. Pat. No. 5,879,918; U.S. Pat. No. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6: 923-926); and LEC1 transformation (WO 00/28058). Also see Weissinger et al. (1988) Ann. Rev. Genet. 22: 421-477; Sanford et al. (1987) Particulate Science and Technology 5: 27-37 (onion); Christou et al. (1988) Plant Physiol. 87: 671-674 (soybean); McCabe et al. (1988) Bio/Technology 6: 923-926 (soybean); Finer and McMullen (1991) In Vitro Cell Dev. Biol. 27P: 175-182 (soybean); Singh et al. (1998) Theor. Appl. Genet. 96: 319-324 (soybean); Datta et al. (1990) Biotechnology 8: 736-740 (rice); Klein et al. (1988) Proc. Natl. Acad. Sci. USA 85: 4305-4309 (maize); Klein et al. (1988) Biotechnology 6: 559-563 (maize); U.S. Pat. Nos. 5,240,855; 5,322,783; and, 5,324,646; Klein et al. (1988) Plant Physiol. 91: 440-444 (maize); Fromm et al. (1990) Biotechnology 8: 833-839 (maize); Hooykaas-Van Slogteren et al. (1984) Nature (London) 311: 763-764; U.S. Pat. No. 5,736,369 (cereals); Bytebier et al. (1987) Proc. Natl. Acad. Sci. USA 84: 5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9: 415-418 and Kaeppler et al. (1992) Theor. Appl. Genet. 84: 560-566 (whisker-mediated transformation); D'Halluin et al. (1992) Plant Cell 4: 1495-1505 (electroporation); Li et al. (1993) Plant Cell Reports 12: 250-255 and Christou and Ford (1995) Annals of Botany 75: 407-413 (rice); Osjoda et al. (1996) Nature Biotechnology 14: 745-750 (maize via Agrobacterium tumefaciens); all of which are herein incorporated by reference.

In specific embodiments, LEC1 coding sequences, AGP inhibitory sequences, FAD (for example, FAD2) inhibitory sequences, and combinations thereof, can be provided to a plant using a variety of transient transformation methods. Such transient transformation methods include, but are not limited to, the introduction of LEC1 coding sequences, AGP inhibitory sequences, FAD inhibitory sequences, and combinations thereof (including variants and fragments) directly into the plant or the introduction of a LEC1 transcript, inhibitory transcripts of AGP expression, inhibitory transcripts of FAD expression, and combinations thereof into the plant. Such methods include, for example, microinjection or particle bombardment. See, for example, Crossway et al. (1986) Mol Gen. Genet. 202: 179-185; Nomura et al. (1986) Plant Sci. 44: 53-58; Hepler et al. (1994) Proc. Natl. Acad. Sci. 91: 2176-2180 and Hush et al. (1994) The Journal of Cell Science 107: 775-784, all of which are herein incorporated by reference. Alternatively, the LEC1 coding sequences, the AGP inhibitory sequences, the FAD inhibitory sequences, and combinations thereof can be transiently transformed into the plant using techniques known in the art. Such techniques include viral vector systems and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, the polynucleotides of interest to practicing the methods of the invention may be introduced into plants by contacting plants with a virus or viral nucleic acids. Generally, such methods involve incorporating one or more nucleotide constructs comprising the polynucleotides of interest within a viral DNA or RNA molecule. It is recognized that LEC1 polypeptides, inhibitory polypeptides targeting AGP expression or function, inhibitory polypeptides targeting FAD (for example, FAD2) expression or function, and combinations thereof may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5: 209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, International Publication Nos. WO 99/25821, WO 99/25854, WO 99/25840, WO 99/25855, and WO 99/25853, all of which are herein incorporated by reference. Briefly, the polynucleotides for use in practicing the invention can be contained in one or more transfer cassettes each of which is flanked by two non-recombinogenic recombination sites. The transfer cassette (or transfer cassettes where multiple cassettes are to be used) is introduced into a plant having stably incorporated into its genome a target site (or target sites where multiple transfer cassettes are to be used) that is flanked by two non-recombinogenic recombination sites that correspond to the sites of the transfer cassette(s). An appropriate recombinase is provided and the transfer cassette(s) is(are) integrated at the target site(s). The polynucleotides of interest are thereby integrated at one or more specific chromosomal positions in the plant genome.

The cells that have been transformed may be grown into plants in accordance with conventional ways. See, for example, McCormick et al. (1986) Plant Cell Reports 5: 81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having the desired combination of polynucleotides stably incorporated into their genome.

Any means for combining the appropriate sequences of the invention into a single plant can be used. It is well known in the art that cassettes can be introduced independently in plants to create independent parental (P1) generations, each with a separate construct. The P1 generations can be crossed to generate filial (F1) generations that have both constructs of interest. Additionally, it is well known in the art that two constructs can be introduced in the same plant to create a P1 generation having both constructs of interest. Finally, it is recognized that F1 strains can also be bred or transformed with at least a third construct having a cassette, gene or interest of a phenotype of interest. These embodiments expressly include any method of generation, whether by transformation or breeding or a combination of both, to create plants having increased oil production when both constructs of interest, i.e., LEC1 expression and AGP inhibition, occur in the same plant. Optionally, LEC1 expression and AGP inhibition can be made to occur in conjunction with inhibition of a fatty acid desaturase (FAD), for example, FAD2, or another gene of interest. Those skilled in the art recognize that the methods and compositions disclosed herein are not limited by the methods of transformation or breeding used to generate the plants or parts thereof (e.g., seed, germ, scutellum, or cotyledon) having the desirable qualities that are disclosed herein.

Pedigree breeding starts with the crossing of two genotypes, such as an elite line of interest and one other elite inbred line having one or more desirable characteristics (i.e., having stably incorporated a polynucleotide of the invention, having a modulated activity and/or level of the polypeptide of the invention, etc) which complements the elite line of interest. If the two original parents do not provide all the desired characteristics, other sources can be included in the breeding population. In the pedigree method, superior plants are selfed and selected in successive filial generations. In the succeeding filial generations, the heterozygous condition gives way to homogeneous lines as a result of self-pollination and selection. Typically in the pedigree method of breeding, five or more successive filial generations of selfing and selection is practiced: F1→F2; F2→F3; F3→F4; F4→F5, etc. After a sufficient amount of inbreeding, successive filial generations will serve to increase seed of the developed inbred. In specific embodiments, the inbred line comprises homozygous alleles at about 95% or more of its loci.

In addition to being used to create a backcross conversion, backcrossing can also be used in combination with pedigree breeding to modify an elite line of interest and a hybrid that is made using the modified elite line. As discussed previously, backcrossing can be used to transfer one or more specifically desirable traits from one line, the donor parent, to an inbred called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, an F1, such as a commercial hybrid, is created. This commercial hybrid may be backcrossed to one of its parent lines to create a BC1 or BC2. Progeny are selfed and selected so that the newly developed inbred has many of the attributes of the recurrent parent and yet several of the desired attributes of the non-recurrent parent. This approach leverages the value and strengths of the recurrent parent for use in new hybrids and breeding.

Therefore, an embodiment of this invention is a method of making a backcross conversion of a maize inbred line of interest, comprising the steps of crossing a plant of a maize inbred line of interest with a donor plant comprising a mutant gene or transgene conferring a desired trait (e.g., a plant expressing LEC1, an AGP inhibitory sequence targeting AGP expression or function, an FAD (for example, FAD2) inhibitory sequence targeting FAD expression or function, or any combination thereof), selecting an F1 progeny plant comprising the mutant gene or transgene conferring the desired trait, and backcrossing the selected F1 progeny plant to the plant of a maize inbred line of interest. This method may further comprise the step of obtaining a molecular marker profile of a maize inbred line of interest and using the molecular marker profile to select for a progeny plant with the desired trait and the molecular marker profile of the inbred line of interest. In the same manner, this method may be used to produce an F1 hybrid seed by adding a final step of crossing the desired-trait conversion of a maize inbred line of interest with a different maize plant to make F1 hybrid maize seed comprising a mutant gene or transgene conferring the desired trait.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, selfed progeny and topcrossing. The selected progeny are cross-pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar. A synthetic cultivar is the resultant progeny formed by the intercrossing of several selected inbreds.

Mass selection is a useful technique when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program.

Mutation breeding is one of many methods that could be used to introduce new traits into an elite line. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays, gamma rays (e.g. cobalt 60 or cesium 137), neutrons (product of nuclear fission by uranium 235 in an atomic reactor), Beta radiation (emitted from radioisotopes such as phosphorus 32 or carbon 14), or ultraviolet radiation (optimally from 2500 to 2900 nm), or chemical mutagens (such as base analogues (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis, the trait may then be incorporated into existing germplasm by traditional breeding techniques, such as backcrossing. Details of mutation breeding can be found in Fehr (1993) Principals of Cultivar Development (Macmillan Publishing Company), the disclosure of which is incorporated herein by reference. In addition, mutations created in other lines may be used to produce a backcross conversion of elite lines that comprises such mutations.

Any breeding or transformation method known in the art, including those discussed in the preceding sections, may be used to reduce or eliminate starch biosynthesis (for example, targeting AGPase activity), fatty acid desaturase activity (for example, targeting FAD2 activity), combinations thereof, and the like. When transformation is used, specific methods that are contemplated include introducing a cassette that encodes a sequence that modulates, eliminates, and/or inhibits starch biosynthesis activity (for example, targeting AGPase activity), fatty acid desaturase activity (for example, targeting FAD2 activity), combinations thereof, and the like. Transformation of a plant with any one cassette or combination of cassettes, as well as introduction of one or more cassettes through breeding may be accomplished by any method. Non-limiting exemplary embodiments are discussed in detail in the following pages.

In certain embodiments, the activity of AGP, FAD, or a combination thereof is reduced or eliminated by transforming a plant cell, a plant embryo, and the like with a cassette that expresses a polynucleotide that inhibits the expression of AGP, FAD, or a combination thereof. The polynucleotide may inhibit the expression of AGP, FAD, or a combination thereof directly, by preventing translation of AGP messenger RNA, FAD messenger RNA, or a combination thereof, or indirectly, by encoding a polypeptide that inhibits the transcription or translation of a plant gene encoding AGP, FAD, or a combination thereof. Methods for inhibiting or eliminating the expression of a gene in a plant are well known in the art, and any such method may be used in the present invention to inhibit the expression of AGP, FAD, or a combination thereof in a plant, a plant embryo, and the like.

In accordance with the present invention, the expression of AGP, FAD, or a combination thereof in a genetically modified plant or plant part thereof is inhibited if the protein level of AGP, FAD, or a combination thereof is statistically lower than the protein level of the same AGP, FAD, or a combination thereof in a plant or plant part thereof that has not been genetically modified or mutagenized to inhibit the expression of that AGP, FAD, or a combination thereof. In particular embodiments of the invention, the protein level of AGP, FAD, or a combination thereof in a plant or plant part thereof modified in accordance with the methods of the present invention is less than 95%, less than 90%, less than 80%, less than 70%, less than 60%, less than 50%, less than 40%, less than 30%, less than 20%, less than 10%, or less than 5% of the protein level of the same AGP, FAD, or a combination thereof in a plant that is not a mutant or that has not been genetically modified to inhibit the expression of that AGP, FAD, or a combination thereof. The expression level of the AGP, FAD, or a combination thereof may be measured directly, for example, by assaying for the level of AGP, FAD, or a combination thereof expressed in the plant or plant part thereof, or indirectly, for example, by measuring the AGPase activity of the AGP protein in the plant or plant part thereof, or by measuring by measuring the desaturase activity of the FAD protein in the plant or plant part thereof. Methods for determining the AGPase activity of AGP and the desaturase activity of FAD are described elsewhere herein.

In other embodiments of the invention, the activity of AGP, FAD, or a combination thereof is reduced or eliminated by transforming a plant cell with a cassette comprising a polynucleotide encoding a polypeptide that inhibits the activity of AGP, FAD, or a combination thereof. The AGPase activity of an AGP or the desaturase activity of an FAD is inhibited according to the present invention if the AGPase activity of AGP or the desaturase activity of FAD is statistically lower the AGPase activity of the same AGP or the desaturase activity of the same FAD in a plant that has not been genetically modified to inhibit the AGPase activity of that AGP or genetically modified to inhibit the desaturase activity of that FAD. In particular embodiments of the invention, the AGPase activity of the AGP in a plant genetically modified according to the invention or the desaturase activity of an FAD of the genetically modified plant (or combinations thereof) is less than 95%, less than 90%, less than 80%, less than 70%, less than 60%, less than 50%, less than 40%, less than 30%, less than 20%, less than 10%, or less than 5% of the AGPase activity of the same AGP or the desaturase activity of the same FAD in a plant that that has not been genetically modified to inhibit the expression of that AGP or FAD, respectively. The AGPase activity of AGP or the desaturase activity of FAD is “eliminated” according to the invention when it is not detectable by the assay methods described elsewhere herein. Methods of determining the AGPase activity of AGP or the desaturase activity of FAD are described elsewhere herein.

In other embodiments, the activity of AGP, FAD, or a combination thereof may be reduced or eliminated by disrupting the gene encoding the AGP protein, the FAD protein, or a combination thereof. The invention encompasses mutagenized plants that carry mutations in AGP, FAD, or a combination thereof, where the mutations reduce expression of the AGP gene, the FAD gene, or a combination thereof or inhibit the AGPase or desaturase activity of the encoded AGP or FAD, respectively.

Thus, many methods may be used to reduce or eliminate the activity of an AGP, an FAD, or a combination thereof. More than one method may be used to reduce the activity of a single AGP gene, FAD gene, or a combination thereof. In addition, combinations of methods may be employed to reduce or eliminate the activity of two or more different AGP or FAD genes. In addition to the general methods discussed supra, further non-limiting examples of methods of reducing or eliminating the expression of AGP, FAD, or other sequences in accordance with the present invention or combinations thereof using a cassette are as follows.

In some embodiments of the invention, inhibition of the expression of AGP, FAD, or combinations thereof may be obtained by sense suppression or cosuppression. For cosuppression, a cassette is designed to express an RNA molecule corresponding to all or part of a messenger RNA encoding AGP, FAD, or a combination thereof in the “sense” orientation. Overexpression of the RNA molecule can result in reduced expression of the native gene. Accordingly, multiple plant lines transformed with the cosuppression expression cassette are screened to identify those that show the greatest inhibition of AGP expression, FAD expression, or a combination thereof.

The polynucleotide used for cosuppression may correspond to all or part of the sequence encoding AGP, FAD, or a combination thereof, all or part of the 5′ and/or 3′ untranslated region of an AGP transcript, a FAD transcript, or a combination thereof, or all or part of both the coding sequence and the untranslated regions of a transcript encoding AGP, FAD, or a combination thereof. In some embodiments where the polynucleotide comprises all or part of the coding region for the AGP, FAD, or a combination thereof, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin et al. (2002) Plant Cell 14: 1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91: 3490-3496; Jorgensen et al. (1996) Plant Mol. Biol. 31: 957-973; Johansen and Carrington (2001) Plant Physiol. 126: 930-938; Broin et al. (2002) Plant Cell 14: 1417-1432; Stoutjesdijk et al (2002) Plant Physiol. 129: 1723-1731; Yu et al. (2003) Phytochemistry 63: 753-763; and U.S. Pat. Nos. 5,034,323, 5,283,184, and 5,942,657; each of which is herein incorporated by reference. The efficiency of cosuppression may be increased by including a poly-dT region in the expression cassette at a position 3′ to the sense sequence and 5′ of the polyadenylation signal. See, U.S. Patent Publication No. 20020048814, herein incorporated by reference. The methods generally involve transforming plants with a nucleotide construct comprising a promoter that is functional in a plant cell operably linked to at least a portion of a nucleotide sequence that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

In some embodiments of the invention, inhibition of the expression of AGP, FAD, or a combination thereof may be obtained by antisense suppression. Antisense constructions, complementary to at least a portion of the messenger RNA (mRNA) for the sequences to be affected can be constructed and inserted in expression cassettes to inhibit function or production of the wild-type gene in a plant. For antisense suppression, the expression cassette is designed to express an RNA molecule complementary to all or part of a messenger RNA encoding the AGP, FAD, or a combination thereof. Overexpression of the antisense RNA molecule can result in reduced expression of the native gene. Accordingly, multiple plant lines transformed with the antisense suppression expression cassette are screened to identify those that show the greatest inhibition of AGP expression, FAD expression, or a combination thereof.

Antisense nucleotides are constructed to hybridize with the corresponding mRNA. The polynucleotide for use in antisense suppression may correspond to all or part of the complement of the sequence encoding AGP, FAD, or a combination thereof, all or part of the complement of the 5′ and/or 3′ untranslated region of the AGP transcript, FAD transcript, or a combination thereof, or all or part of the complement of the coding sequence and the untranslated regions of a transcript encoding AGP, FAD, or a combination thereof. In addition, the antisense polynucleotide may be fully complementary (i.e., 100% identical to the complement of the target sequence) or partially complementary (i.e., less than 100% identical to the complement of the target sequence) to the target sequence. Modifications of the antisense sequences may be made as long as the sequences hybridize to and interfere with expression of the corresponding miRNA. In this manner, antisense constructions having 70%, optimally 80%, more optimally 85% sequence identity to the corresponding antisensed sequences may be used.

Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu et al. (2002) Plant Physiol. 129: 1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. Patent Publication No. 20020048814, herein incorporated by reference.

In some embodiments of the invention, inhibition of the expression of AGP, FAD, or a combination thereof may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of expression of AGP, FAD, or a combination thereof. Methods for using dsRNA interference to inhibit the expression of endogenous plant genes are described in Waterhouse et al. (1998) Proc. Natl. Acad. Sci. USA 95: 13959-13964; Liu et al. (2002) Plant Physiol. 129: 1732-1743; and International Publication Nos. WO 99/49029, WO 99/53050, WO 99/61631, WO 99/32619 and WO 00/49035; each of which is herein incorporated by reference.

In some embodiments of the invention, inhibition of the expression of AGP, FAD, or a combination thereof may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 4: 29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the protein (i.e., an AGP, FAD, or a combination thereof) whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, the methods for using hpRNA interference to inhibit or silence the expression of genes described in Chuang and Meyerowitz (2000) Proc. Natl. Acad. Sci. USA 97: 4985-4990; Stoutjesdijk et al. (2002) Plant Physiol. 129: 1723-1731; Waterhouse and Helliwell (2003) Nat. Rev. Genet. 4: 29-38; Pandolfini et al. BMC Biotechnology 3: 7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30: 135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407: 319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407: 319-320; Wesley et al. (2001) Plant J. 27: 581-590; Wang and Waterhouse (2001) Curr. Opin. Plant Biol. 5: 146-150; Waterhouse and Helliwell (2003) Nat. Rev. Genet. 4: 29-38; Helliwell and Waterhouse (2003) Methods 30: 289-295, and U.S. Patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, International Publication No. WO 02/00904, herein incorporated by reference.

Inhibition of the expression of AGP, FAD, or a combination thereof can also be achieved with amplicons. Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for AGP, FAD, or a combination thereof). Methods of using amplicons to inhibit the expression of endogenous plant genes are described, for example, in Angell and Baulcombe (1997) EMBO J. 16: 3675-3684, Angell and Baulcombe (1999) Plant J. 20: 357-362, and U.S. Pat. No. 6,646,805, each of which is herein incorporated by reference.

In some embodiments, the polynucleotide expressed by the expression cassette of the invention is catalytic RNA or has ribozyme activity specific for the messenger RNA of AGP, FAD, or a combination thereof. Thus, the polynucleotide causes the degradation of the endogenous messenger RNA, resulting in reduced expression of AGP, FAD, or a combination thereof. This method is described, for example, in U.S. Pat. No. 4,987,071, herein incorporated by reference.

In other embodiments of the invention, inhibition of the expression of AGP, expression of FAD, or a combination thereof may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425: 257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of expression of AGP, expression of FAD, or a combination thereof, the 22-nucleotide sequence is selected from an AGP transcript sequence, a FAD transcript sequence, or a combination thereof and contains 22 nucleotides of said AGP sequence, said FAD sequence, or a combination thereof in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

Nucleotide sequences encoding variant polypeptides (e.g., AGP1, AGP2 or FAD2) can also be used in dominant-negative strategies to reduce a particular biological activity within an organism or cell thereof. Such dominant-negative strategies are known in the art and can involve the expression of a modified subunit of a multisubunit protein. Generally, such a modified subunit comprises a polypeptide that is able to affect, or interact with, other members of the multisubunit protein complex and thereby reduce, or eliminate, the biological activity of the complex. It is recognized that such dominant-negative strategies can be used to reduce or eliminate the activity of both homomeric enzymes and heteromeric enzymes. “Homomeric enzyme” is intended to mean an enzyme that is comprised of two or more subunits each having the same amino acid sequences. “Heteromeric enzyme” is intended to mean an enzyme that is comprised of two or more subunits wherein not all the subunits comprise the same amino acid sequence. While the methods of the invention do not depend on a particular biological mechanism, typically such a dominant-negative approach will involve the expression of a variant of a polypeptide of the invention that does not possess the complete biological activity of the native polypeptide. It is recognized that such a dominant-negative approach does not depend on eliminating or reducing the expression of native genes in a plant, only that such an approach involves the expression of nucleotide sequence of the invention that encodes a variant polypeptide that is capable of causing a reduction or elimination of the desired biological activity in a plant or cell thereof.

Similar results to those of the dominant negative mutants can be achieved through deletion or mutation of the wild-type gene. Those skilled in the art recognize that such deletions and mutation can be accomplished in many ways, including targeted deletion or mutation of the wild-type nucleotide sequences with such vectors as Flp-Frt transposons (see, for example, U.S. Pat. No. 6,455,315, herein incorporated by reference in its entirety), spliceosome-mediated chimerism (see, for example, U.S. Pat. No. 6,083,702), as well as selection of plants expressing natural field mutations and deletions, or chemically induced mutations and deletions of the genes of interest.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an miRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

In one embodiment, the polynucleotide to be introduced into the plant comprises an inhibitory sequence that encodes a zinc finger protein that binds to a gene encoding AGP, FAD, or a combination thereof, resulting in reduced expression of the gene. In particular embodiments, the zinc finger protein binds to a regulatory region of an AGP gene, an FAD gene, or a combination thereof. In other embodiments, the zinc finger protein binds to a messenger RNA encoding AGP, encoding FAD, or a combination thereof and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 20030037355; each of which is herein incorporated by reference.

In some embodiments of the invention, the polynucleotide comprises an inhibitory sequence that encodes an antibody that binds to at least one isoform of AGP, FAD, or a combination thereof, and reduces the AGPase activity of AGP or the desaturase activity of an FAD, respectively. In another embodiment, the binding of the antibody results in increased turnover of the antibody-antigen complex (comprising AGP protein, or FAD protein, or a combination thereof) by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21: 35-36, incorporated herein by reference.

In yet other embodiments of the invention, the polynucleotide comprises an inhibitory sequence that encodes a polypeptide that specifically inhibits the AGPase activity of AGP or the desaturase activity of a FAD (for example, FAD2).

In some embodiments of the present invention, the activity of AGP, an FAD, or a combination thereof is reduced or eliminated by disrupting the gene encoding the AGP, the FAD, or a combination thereof. The genes encoding AGP, an FAD, or a combination thereof may be disrupted by any method known in the art. For example, in one embodiment, the gene is disrupted by transposon tagging. In another embodiment, the gene is disrupted by mutagenizing plants using random or targeted mutagenesis, and selecting for plants that have reduced AGP or FAD activity.

In one embodiment of the invention, transposon tagging is used to reduce or eliminate the AGPase activity of AGP, the desaturase activity of an FAD, or a combination thereof. Transposon tagging comprises inserting a transposon within an endogenous AGP gene, an endogenous FAD gene, or a combination thereof to reduce or eliminate expression of AGP, an FAD, or a combination thereof “AGP gene” is intended to mean the gene that encodes an AGP protein, such as AGP1, AGP2, or a combination thereof according to the invention. “FAD gene” is intended to mean the gene that encodes an FAD protein, such as FAD2, according to the invention.

In this embodiment, the expression of AGP, FAD, or a combination thereof is reduced or eliminated by inserting a transposon within a regulatory region or coding region of the gene encoding the AGP protein, the FAD protein, or a combination thereof A transposon that is within an exon, intron, 5′ or 3′ untranslated sequence, a promoter, or any other regulatory sequence of the AGP gene, the FAD gene, or a combination thereof may be used to reduce or eliminate the expression and/or activity of the encoded AGP protein, the FAD protein, or a combination thereof.

Methods for the transposon tagging of specific genes in plants are well known in the art. See, for example, Maes et al. (1999) Trends Plant Sci. 4: 90-96; Dharmapuri and Sonti (1999) FEMS Microbiol. Lett. 179: 53-59; Meissner et al. (2000) Plant J. 22: 265-274; Phogat et al. (2000) J. Biosci. 25: 57-63; Walbot (2000) Curr. Opin. Plant Biol. 2: 103-107; Gai et al. (2000) Nucleic Acids Res. 28: 94-96; Fitzmaurice et al. (1999) Genetics 153: 1919-1928). In addition, the TUSC process for selecting Mu insertions in selected genes has been described in Bensen et al. (1995) Plant Cell 7: 75-84; Mena et al (1996) Science 274: 1537-1540; and U.S. Pat. No. 5,962,764; each of which is herein incorporated by reference.

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see Ohshima et al. (1998) Virology 243: 472-481; Okubara et al. (1994) Genetics 137: 867-874; and Quesada et al. (2000) Genetics 154: 421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See McCallum et al. (2000) Nat. Biotechnol. 18: 455-457, herein incorporated by reference.

In another embodiment of this invention, dominant mutants can be used to trigger RNA silencing due to gene inversion and recombination of a duplicated gene locus. See, for example, Kusaba et al. (2003) Plant Cell 15: 1455-1467.

The invention encompasses additional methods for reducing or eliminating the activity of AGP, an FAD, or combinations thereof. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham et al. (1999) Proc. Natl. Acad. Sci. USA 96: 8774-8778; each of which is herein incorporated by reference.

Mutations that impact gene expression or that interfere with AGPase activity, FAD desaturase activity, or combinations thereof of the encoded AGP and FAD proteins are well known in the art. Insertional mutations in gene exons usually result in null-mutants. Mutations in conserved residues are particularly effective in inhibiting the AGPase activity of the encoded AGP protein, the FAD desaturase activity of the encoded FAD protein, or combinations thereof. Such mutants can be isolated according to well-known procedures, and mutations in different AGP genetic loci and FAD genetic loci can be stacked by genetic crossing. See, for example, Gruis et al. (2002) Plant Cell 14: 2863-2882.

Thus, in certain embodiments the polynucleotides for use in the methods of the present invention can be “stacked” with any combination of nucleic acids of interest in order to create plants with a desired phenotype. By “stacked” or “stacking” is intended that a plant of interest contains one or more nucleic acids collectively comprising multiple nucleotide sequences so that the transcription and/or expression of multiple genes are altered in the plant.

Constructs comprising a LEC1-encoding polynucleotide and a polynucleotide comprising an AGP inhibitory sequence (and, optionally, a polynucleotide comprising an FAD inhibitory sequence) also can be stacked with any other polynucleotide(s) to produce plants having a variety of desired trait combinations including, for example, traits desirable for animal feed such as high oil genes (see, e.g., U.S. Pat. No. 6,232,529, which is incorporated herein by reference); balanced amino acids (e.g., hordothionins; see U.S. Pat. Nos. 5,990,389; 5,885,801; 5,885,802; and 5,703,409, each of which is incorporated herein by reference); barley high lysine (Williamson et al. (1987) Eur. J. Biochem. 165: 99-106, International Publication Nos. WO 98/20122 and WO 98/20133); high methionine proteins (Pedersen et al. (1986) J. Biol. Chem. 261: 6279; Kirihara et al. (1988) Gene 71: 359; and Musumura et al. (1989) Plant Mol. Biol. 12: 123); increased digestibility (e.g., modified storage proteins) and thioredoxins (U.S. Ser. No. 10/005,429, filed Dec. 3, 2001).

Constructs comprising a LEC1-encoding polynucleotide and a polynucleotide comprising an AGP inhibitory sequence (and, optionally, a polynucleotide comprising an FAD inhibitory sequence) also can be stacked with one or more polynucleotides encoding a desirable trait such as a polynucleotide that confers, for example, insect, disease, or herbicide resistance (e.g., Bacillus thuringiensis toxic proteins; U.S. Pat. Nos. 5,366,892; 5,747,450; 5,737,514; 5,723,756; 5,593,881; Geiser et al. (1986) Gene 48: 109); lectins (Van Damme et al. (1994) Plant Mol. Biol. 24: 825); fumonisin detoxification genes (U.S. Pat. No. 5,792,931); avirulence and disease resistance genes (Jones et al. (1994) Science 266: 789; Martin et al. (1993) Science 262: 1432; Mindrinos et al. (1994) Cell 78: 1089); acetolactate synthase mutants that lead to herbicide resistance such as the S4 and/or Hra mutations; inhibitors of glutamine synthase such as phosphinothricin or basta (e.g., the bar gene); and glyphosate resistance (e.g., the EPSPS gene and the GAT gene; see, for example, U.S. Publication No. 20040082770 and WO 03/092360). Additional polynucleotides that can be stacked include, for example, those encoding traits desirable for processing or process products such as modified oils (e.g., fatty acid desaturase genes (U.S. Pat. No. 5,952,544; International Publication No. WO 94/11516); modified starches (e.g., AGPases, starch synthases, starch branching enzymes, and starch debranching enzymes); modified cell wall amounts and/or properties (e.g., UDP-glucose dehydrogenase (U.S. Pat. No. 6,399,859), Reversibly Glycosylated Protein (RGP) (U.S. Pat. No. 6,194,638)); and polymers or bioplastics (e.g., U.S. Pat. No. 5,602,321). Constructs comprising a LEC1-encoding polynucleotide and a polynucleotide comprising an AGP inhibitory equence (and, optionally, a polynucleotide comprising an FAD inhibitory sequence) also can be stacked with one or more polynucleotides that provide desirable agronomic traits such as male sterility (e.g., U.S. Pat. No. 5,583,210), stalk strength, flowering time, or transformation technology traits such as cell cycle regulation or gene targeting (e.g., International Publication Nos. WO 99/61619; WO 00/17364; WO 99/25821). Other desirable traits that are known in the art include high oil content; increased digestibility; balanced amino acid content; and high energy content. Such traits may refer to properties of both seed and non-seed plant tissues, or to food or feed prepared from plants or seeds having such traits.

These stacked combinations can be created by any method including, but not limited to, cross breeding plants. If traits are stacked by genetically transforming the plants, the constructs comprising one or more of the polynucleotides of interest can be combined at any time and in any order. Similarly, where a method requires more than one step to be performed, it is understood that steps may be performed in any order that accomplishes the desired end result. For example, a transgenic plant comprising one or more desired traits can be used as the target to introduce further traits by subsequent transformation. The traits can be introduced simultaneously in a co-transformation protocol with the polynucleotides of interest provided by any combination of cassettes suitable for transformation. For example, if two sequences will be introduced, the two sequences can be contained in separate cassettes (trans) or contained on the same transformation cassette (cis). Transcription and/or expression of the sequences can be driven by the same promoter or by different promoters. In certain cases, it may be desirable to introduce a cassette that will suppress the expression of the polynucleotide of interest. This may be combined with any combination of other cassettes to generate the desired combination of traits in the plant. Alternatively, traits may be stacked by transforming different plants to obtain those traits; the transformed plants may then be crossed together, and progeny containing all of the desired traits may be selected.

Stacking may also be performed with fragments of a particular gene or nucleic acid. In such embodiments, a plants is transformed with at least one fragment and the resulting transformed plant is crossed with another transformed plant; progeny of this cross may then be selected which contain the fragment in addition to other transgenes, including, for example, other fragments. These fragments may then be recombined or otherwise reassembled within the progeny plant, for example, using site-specific recombination systems known in the art. Such stacking techniques could be used to provide any property associated with fragments, including, for example, hairpin RNA (hpRNA) interference or intron-containing hairpin RNA (ihpRNA) interference.

As used herein, the term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The compositions and methods of the present invention can be used to manipulate lipid and starch biosynthesis and thus alter oil phenoptype of any plant species of interest, including, but not limited to, monocots and dicots. Plants of particular interest include grain plants that provide seeds of interest, oilseed plants, and leguminous plants. Seeds of interest include grain seeds, such as corn (Zea mays), wheat, barley, rice, sorghum, rye, etc. Oilseed plants include soybean, Brassica, safflower, sunflower, cotton, maize, peanut, alfalfa, palm, sesame, coconut, etc. Leguminous plants include beans and peas. Beans include soybean, guar, locust bean, fenugreek, garden beans, cowpea, mungbean, lima bean, fava bean, lentils, chickpea, etc.

The following examples are offered by way of illustration and not by way of limitation.

### EXPERIMENTAL

### Example 1

### Generation of High Oil Content Seed via Simultaneous Expression of LEC1 and Suppression of AGP2

F₁ kernels from a single ear that, per se, all contained the LEC1 transgene (SEQ ID NO:1, encoding SEQ ID NO:2) and also segregated 1:1 for the AGP2 transgene (SEQ ID NO:5, encoding truncated AGP2 amino acid sequence of SEQ ID NO:6), were planted. The seed represented the cross of a single AGP2 event (i.e., AGP2-cosuppression) with a single LEC1 event (i.e., LEC1 expression). The pedigree and per se segregation pattern indicated that all kernels were heterozygous for the LEC1 transgene and half were also heterozygous for the AGP2 transgene. Ten plants containing LEC1 alone and ten plants having both transgenes were self-pollinated. Upon analyzing 24 mature embryos from each ear for embryo oil level, the presence of both genes was more favorable for increasing oil concentration than LEC1 alone. Three F₁ plants that were heterozygous for both AGP2 and LEC1 transgenes were self-pollinated to produce an F2 generation with definable genotypic and phenotypic frequencies of all combinations of wild-type, heterozygous, and homozygous states for each transgene among embryos of an ear as shown in Table 1(A) and 1(B).

TABLE 1(A)

| | | Lec 1 genotypes | | | |
|---|---|---|---|---|---|
| | | to Expect Among | | | |
| | | Single Seed | | | |
| | | +/+ | +/L | L/+ | L/L |
| Agp2 | +/+ | +/+ | +/+ | +/+ | +/+ |
| Genotypes to | | +/+ | +/L | L/+ | L/L |
| Expect Among | +/A | +/A | +/A | +/A | +/A |
| Single Seed | | +/+ | +/L | L/+ | L/L |
| | A/+ | A/+ | A/+ | A/+ | A/+ |
| | | +/+ | +/L | L/+ | L/L |
| | A/A | A/A | A/A | A/A | A/A |
| | | +/+ | +/L | L/+ | L/L |

TABLE 1(B)

| Expected | | | |
|---|---|---|---|
| Phenotypic Classes | | | |
| Agp2 | Lec1 | Freq. | Freq (%) |
| Wt | Wt | 1/16 | 6 |
| Wt | Hetero | 2/16 | 13 |
| Wt | Homo | 1/16 | 6 |
| Hetero | Wt | 2/16 | 13 |
| Hetero | Hetero | 4/16 | 25 |
| Hetero | Homo | 2/16 | 13 |
| Homo | Wt | 1/16 | 6 |
| Homo | Hetero | 2/16 | 13 |
| Homo | Homo | 1/16 | 6 |

Approximately 200 mature embryos were collected from each ear and tested for dry weight, oil, and zygosity of AGP2 and LEC1 transgenes. On average:
1) all genotypic classes occurred at the expected frequencies within each ear (see FIGS. 1 through 5);
2) the efficacy of the transgenes, alone or in combination, in affecting traits was the same in heterozygous and homozygous seed, indicating that these genes display dominant gene action (see FIGS. 1 through 5); and
3) the combination of AGP2 and LEC1 transgenes consistently increased oil level above that expected from an additive effect (see FIGS. 1 through 4).

A significantly higher concentration of oil is produced when the two transgenes are present simultaneously (Table 2A and 2B). The data were tested with two analyses of variance (ANOVA) models: one that treats individual kernels (Reduced Model) and another that treats individual ears (Full Model) as the experimental unit (Table 2C). Using the Full Model, when either AGP2 or LEC1 transgenes are individually present in the plant, there is a significant increase in the concentration of oil in the embryo. Even greater evidence of this statistically significant result is obtained with the Reduced Model. These seeds may further be used for cross-breeding to impart a third desirable trait such as inhibition of FAD2 expression.

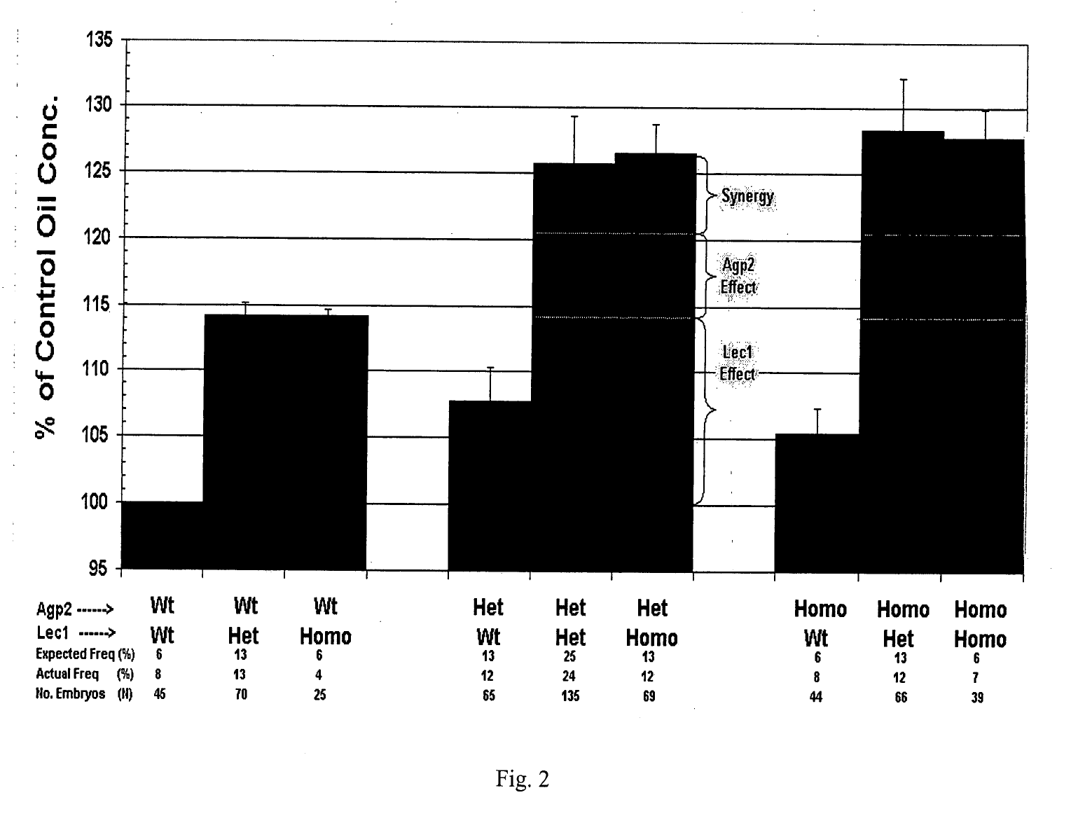

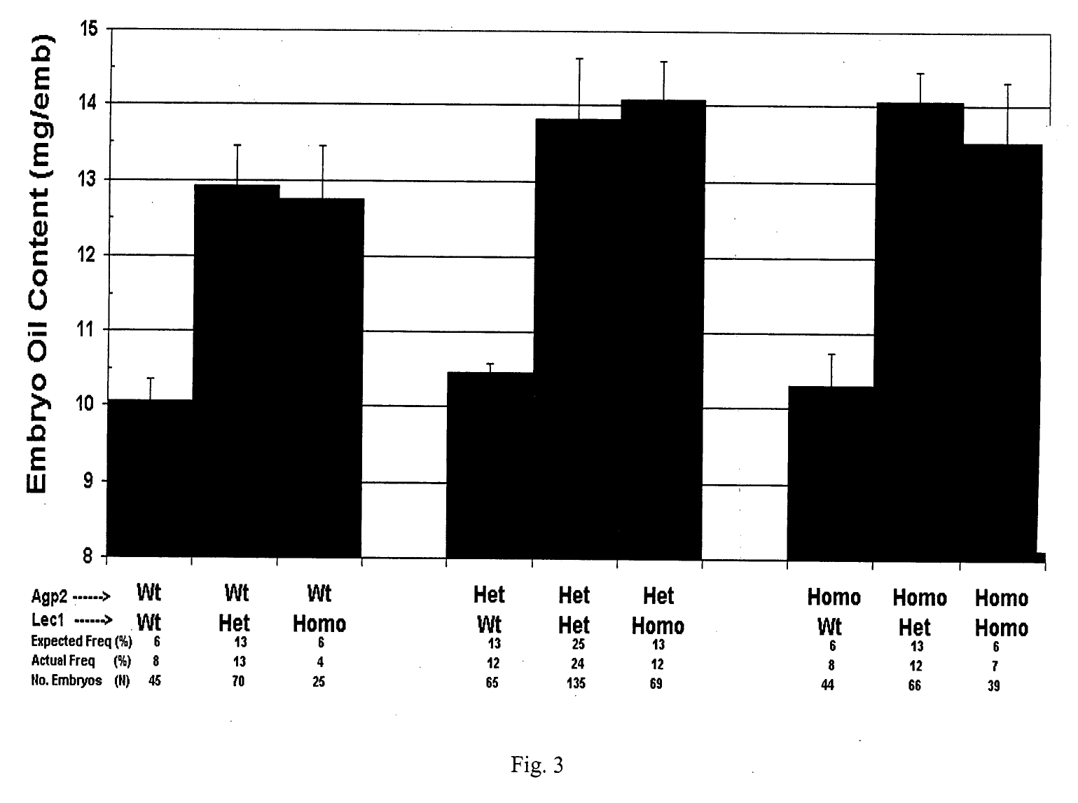

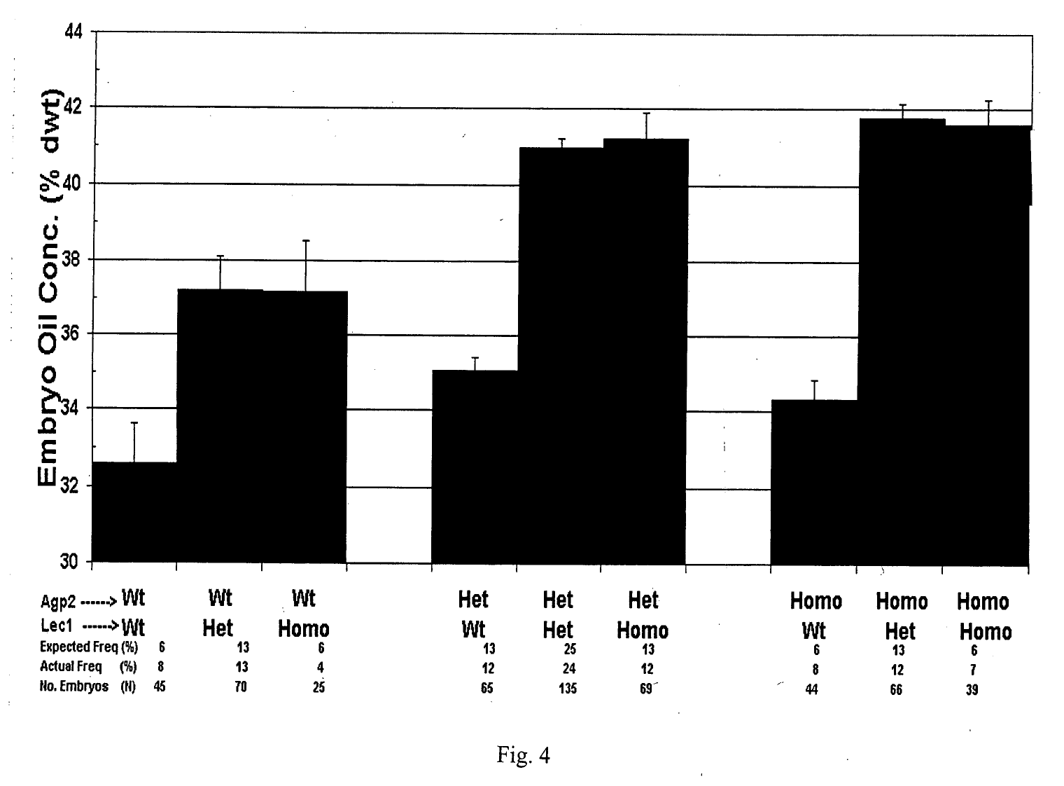

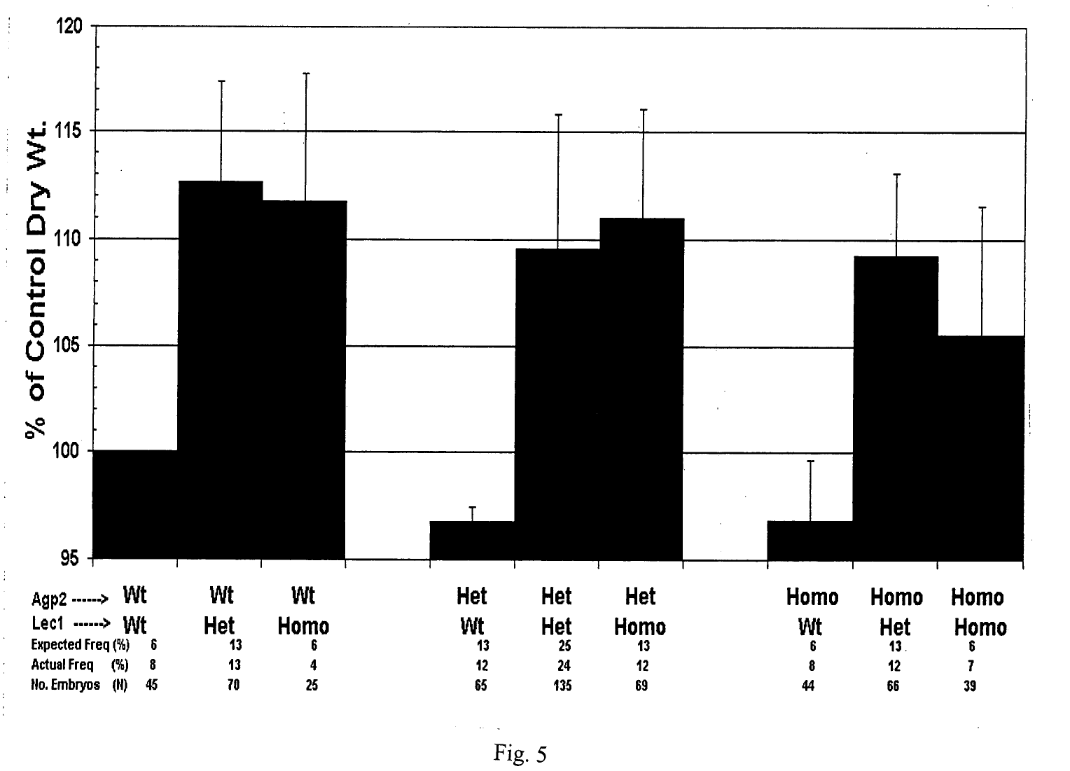

TABLE 2
(A)

| | zygosity | zygosity | zygosity | zygosity | zygosity | zygosity | zygosity | zygosity | zygosity |
|---|---|---|---|---|---|---|---|---|---|
| Agp2 | Wt | Wt | Wt | Het | Het | Het | Homo | Homo | Homo |
| Lec1 | Wt | Het | Homo | Wt | Het | Homo | Wt | Het | Homo |
| Expected Freq (%) | 6 | 13 | 6 | 13 | 25 | 13 | 6 | 13 | 6 |
| Actual Freq (%) | 8 | 13 | 4 | 12 | 24 | 12 | 8 | 12 | 7 |
| No. Embryos (N) | 45 | 70 | 25 | 65 | 135 | 69 | 44 | 66 | 39 |
| % of Control Oil Content (FIG. 1) | 100 | 128 | 127 | 104 | 138 | 140 | 103 | 140 | 134 |
| % of Control Oil Conc. (FIG. 2) | 100 | 114 | 114 | 108 | 126 | 127 | 105 | 128 | 128 |
| Embryo Oil Content (mg/emb) (FIG. 3) | 10 | 13 | 12.75 | 10.5 | 13.75 | 14 | 10.5 | 14 | 13.5 |
| Embryo Oil Conc. (% dwt) (FIG. 4) | 32.5 | 37 | 37 | 35 | 41 | 41.25 | 34.25 | 41.75 | 41.5 |
| % of Control Dry Wt. (FIG. 5) | 100 | 112.75 | 112 | 97 | 109.5 | 111 | 97 | 109.5 | 105.5 |

(B)

| | Embryo Oil Concentration | | | Embryo Oil | | | Embryo Dry Weight | | |
|---|---|---|---|---|---|---|---|---|---|
| | (% dwt) | | | Content (mg) | | | (mg) | | |
| Embryo | | | % of | | | % of | | | % of |
| Stage | Control | Cosup. | Control | Control | Cosup. | Control | Control | Cosup. | Control |
| Mat | 25.1 | 30.6 | 122 | 9.2 | 10.2 | 112 | 36.6 | 33.3 | 91 |

(C)

| | Num | Den | | |
|---|---|---|---|---|
| Effect | DF | DF | F Value | Pr > F |
| Agp2 | 2 | 16 | 55.9 | <.0001 |
| Lec1 | 2 | 16 | 174.9 | <.0001 |
| Agp2 × Lec1 | 4 | 16 | 2.98 | .05 |

### Example 2

### Cloning of ADP-Glucose Pyrophosphorylase Gene (AGP), Construction of Expression Vectors, and Transformation and Regeneration of Plants

A full-length cDNA clone corresponding to the AGP1 subunit of the embryo isoform of ADP-glucose pyrophosphorylase (Giroux et al. (1995) Plant Physiol., 108: 1333-1334; incorporated herein in its entirety by reference) is obtained using the polymerase chain reaction (PCR). A template is prepared by generating first strand cDNA from total RNA isolated from 16 day-old maize kernels. Primers are designed based upon the published sequence of AGP1. These primers are used in a PCR reaction to amplify the AGP1 cDNA by conventional methods. The resulting PCR product is purified and subcloned into the vector pCRII (Invitrogen) and sequenced on both strands to confirm its identity. This clone is designated p9734. An embryo-specific expression cassette is constructed by digesting p9734 with EcoRI, treating with Klenow enzyme to generate blunt ends, and then gel purifying the resulting approximately 1.6 kb AGP1 cDNA. This is ligated between the globulin-1 (glb1) promoter and terminator sequences of the vector p3303. Extensive restriction enzyme mapping is performed on the resulting clone, p9733, to ensure the AGP1 cDNA is in an antisense orientation relative to the glb1 promoter and terminator sequences. In preparation for maize transformation, p9733 is linked in cis to two separate selectable marker expression cassettes by subsequent ligations with p3528 (2× CAMV (Cauliflower mosaic virus promoter)::BAR (phosphinothricin acetyltransferase gene)::PinII (protease inhibitor II terminator) and p8092 (Ubi (ubiquitin promoter)::PAT (phosphinothricin-N-acetyltransferase)::35S (CAMV 35S terminator)) to generate plasmids p10000 and p9763, respectively. These plasmids are used in particle bombardment of maize immature embryos.

The general method of genetic transformation used to produce transgenic maize plants is mediated by bombardment of embryogenically responsive immature embryos with tungsten particles associated with DNA plasmids that comprise a selectable and an unselectable marker gene.

(A) Preparation of Tissue

Immature embryos of “High Type II” are the targets for particle bombardment-mediated transformation. This genotype is the F₁ of two purebred genetic lines, parent A and parent B, derived from A188×B73. Both parents are selected for high competence of somatic embryogenesis. See, Armstrong et al. (1991), Maize Genetics Cooperation Newsletter, 65: 92; incorporated herein in its entirety by reference.

Ears from F₁ plants are selfed or sibbed, and embryos are aseptically dissected from developing caryopses when the scutellum first becomes opaque. The proper stage occurs about 9-13 days post-pollination, and most generally about 10 days post-pollination, and depends on growth conditions. The embryos are about 0.75 to 1.5 mm long. Ears are surface sterilized with 20-50% Clorox for 30 min, followed by 3 rinses with sterile distilled water.

Immature embryos are cultured, scutellum oriented upward, on embryogenic induction medium comprised of N6 basal salts (Chu et al. (1975) Scientia Sinica (Peking) 18: 659-668; incorporated herein in its entirety by reference; Eriksson vitamins (see, Ericksson (1965) Physiol. Plant 18: 976-993; incorporated herein in its entirety by reference), 0.5 mg/l thiamine HCL, 30 gm/l sucrose, 2.88 gm/l L-proline, 1 mg/l 2,4-dichlorophenoxyacetic acid, 2 gm/l Gelrite, and 8.5 mg/l AgNO₃. The medium is sterilized by autoclaving at 121° C. for 15 min and dispensed into 100×25 mm petri dishes. AgNO₃ is filter-sterilized and added to the medium after autoclaving. The tissues are cultured in complete darkness at 28° C. After about 3 to 7 days, most usually about 4 days, the scutellum of the embryo has swelled to about double its original size and the protuberances at the coleorhizal surface of the scutellum indicate the inception of embryogenic tissue. Up to 100% of the embryos display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per petri dish are located in the center of a petri dish in an area about 2 cm in diameter. The embryos are maintained on this medium for 3-16 hours in complete darkness at 28° C. just prior to bombardment with particles associated with plasmid DNAs containing the selectable and unselectable marker genes.

To effect particle bombardment of embryos, the particle-DNA agglomerates are accelerated using a DuPont PDS-1000 particle acceleration device. The particle-DNA agglomeration is briefly sonicated and 10 μl are deposited on macrocarriers and the ethanol allowed to evaporate. The macrocarrier is accelerated onto a stainless-steel stopping screen by the rupture of a polymer diaphragm (rupture disk). Rupture is effected by pressurized helium. Depending on the rupture disk breaking pressure, the velocity of particle-DNA acceleration may be varied. Rupture disk pressures of 200 to 1800 psi are commonly used, with those of 650 to 1100 psi being more preferred, and about 900 psi being most highly preferred. Rupture disk breaking pressures are additive so multiple disks may be used to effect a range of rupture pressures.

Generally, the shelf containing the plate with embryos is 5.1 cm below the bottom of the macrocarrier platform (shelf #3), but may be located at other distances. To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A petri dish with the target embryos is placed into the vacuum chamber and located in the projected path of accelerated particles. A vacuum is created in the chamber, generally about 28 inches Hg. After operation of the device, the vacuum is released and the petri dish is removed.

Bombarded embryos remain on the osmotically adjusted medium during bombardment, and generally for two days subsequently, although the embryos may remain on this medium for 1 to 4 days. The embryos are transferred to selection medium comprised of N6 basal salts, Eriksson vitamins, 0.5 mg/l thiamine HCL, 30 gm/l sucrose, 1 mg/l 2,4-dichlorophenoxyacetic acid, 2 gm/l Gelrite, 0.85 mg/l AgNO₃ and 3 mg/l bialaphos. Bialaphos is added, filter-sterilized. The embryos are subcultured to fresh selection medium at 10 to 14 day intervals. After about 7 weeks, embryogenic tissue, putatively transformed for both selectable and unselected marker genes, is seen to proliferate from about 7% of the bombarded embryos. Putative transgenic tissue is rescued, and that tissue derived from individual embryos is considered to be an event and is propagated independently on selection medium. Two cycles of clonal propagation is achieved by visual selection for the smallest contiguous fragments of organized embryogenic tissue.

For regeneration of transgenic plants, embryogenic tissue is subcultured to medium comprised of MS salts and vitamins (Murashige and Skoog (1962) Physiologia Plantarum 15: 473-497; 1962; incorporated herein in its entirety by reference), 100 mg/l myo-inositol, 60 gm/l sucrose, 3 gm/l Gelrite, 0.5 mg/l zeatin, 1 mg/l indole-3-acetic acid, 26.4 ng/l cis-trans-abscissic acid, and 3 mg/l bialaphos in 100×25 mm petri dishes and incubated in darkness at 28° C. until the development of well-formed, matured somatic embryos can be visualized. This requires about 14 days. Well-formed somatic embryos are opaque and cream-colored, and are comprised of an identifiable scutellum and coleoptile. The embryos are individually subcultured to germination medium comprised of MS salts and vitamins, 100 mg/l myo-inositol, 40 gm/l sucrose and 1.5 gm/l Gelrite in 100×25 mm petri dishes and incubated under a 16 hr light:8 hr dark photoperiod and 40 μEinsteins m⁻² sec⁻¹ from cool-white fluorescent tubes. After about 7 days, the somatic embryos have germinated and produced a well-defined shoot and root. The individual plants are subcultured to germination medium in 125×25 mm glass tubes to allow further plant development. The plants are maintained under a 16 hr light:8 hr dark photoperiod and 40 μEinsteinm⁻²sec⁻¹ from cool-white fluorescent tubes. After about 7 days, the plants are well-established and are transplanted to horticultural soil, hardened off, and potted into commercial greenhouse soil mixture and grown to sexual maturity in a greenhouse. An elite inbred line is used as a male to pollinate regenerated transgenic plants.

(B) Preparation of Particles

Fifteen mg of tungsten particles (General Electric), 0.5 to 1.8 μm, optimally 1 to 1.8 μm, and most optimally 1 μm, are added to 2 ml of concentrated nitric acid. This suspension is sonicated at 0° C. for 20 min (Branson Sonifier Model 450, 40% output, constant duty cycle). Tungsten particles are pelleted by centrifugation at 10,000 rpm (Biofuge) for 1 min and the supernatant is removed. Two ml of sterile distilled water is added to the pellet and sonicated briefly to resuspend the particles. The suspension is pelleted, and 1 ml of absolute ethanol is added to the pellet and sonicated briefly to resuspend the particles. The particles are rinsed, pelleted, and resuspend 2 more times with sterile distilled water, and finally the particles are resuspend in 2 ml of sterile distilled water. The particles are subdivided into 250 μl aliquots and stored frozen.

(C) Preparation of Particle-Plasmid DNA Association

The stock of tungsten particles is sonicated briefly in a water bath sonicator (Branson Sonifier Model 450, 20% output, constant duty cycle) and 50 μl is transferred to a microfuge tube. Plasmid DNA is added to the particles for a final DNA amount of 0.1 to 10 μg in 10 μl total volume, and briefly sonicated. Generally, 1 μg total DNA is used. Specifically, 10 μl of p9764 (ubiₚ::ubiint::MO-PAT(maize optimized PAT))::CaMV35sₜ+glb1ₚ::antisense-AGP1::glb1ₜ) or 10 μl of p9763 (CaMV35sₜ::MO-PAT::ubiint::ubiₚ+glb1ₚ::antisense-AGP1::glb1ₜ),at 0.1 μg/μl in TE buffer, are added to the particle suspension. Fifty μl of sterile aqueous 2.5 M CaCl₂ are added, and the mixture is briefly sonicated and vortexed. Twenty μl of sterile aqueous 0.1 M spermidine are added and the mixture is briefly sonicated and vortexed. The mixture is incubated at room temperature for 20 minutes with intermittent brief sonication. The particle suspension is centrifuged, and the supernatant is removed. Two hundred fifty μl of absolute ethanol is added to the pellet and briefly sonicated. The suspension is pelleted, the supernatant is removed, and 60 μl of absolute ethanol is added. The suspension is sonicated briefly before loading the particle-DNA agglomeration onto macrocarriers.

A sample of tissue from each event is processed to recover DNA. The DNA is probed with primer sequences designed to amplify DNA sequences overlapping the glb1 promoter and the AGP1 portion of the plasmid and/or the glb1 terminator and the AGP1 portion of the plasmid. Embryogenic tissue with amplifiable sequence is advanced to plant regeneration.

Seed of mature ears from transgenic plants shown to be PCR positive are harvested and dried to a similar moisture concentration of approximately 12%.

More critical determination of how antisense expression of AGP1 in the germ impacts seed metabolism and chemical composition is conducted by studies involving isolated germs. Seed is harvested at approximately 25 days after pollination (DAP) and germ isolated by dissection. The activity of ADP-glucose pyrophosphorylase (ATP:D-Glc-1-phosphate adenyltransferase, EC 2.7.7.27) is measured in isolated, fresh, diced embryos by extraction (4° C.; Virtishear Homogenizer, 25,000 rpm, 20 sec) in buffer [1:10 wt/vol; 50 mM Hepes-NaOH (pH 7.5), 5 mM MgCl₂, 1 mM DTT, 1 mg/mL BSA]. The homogenate is centrifuged (30,000×g, 15 min, 4° C.) and the supernatant assayed for activity as described in Singletary et al. (1980) Plant Physiol. 92: 160-167; incorporated herein in its entirety by reference. The supernatant is also run on 4-20% SDS-PAGE gels and blotted to nitrocellulose. A Western assay for detection of AGP1 protein is conducted using an antibody produced against a portion of the AGP1 protein. Methods for blotting and western development are followed according to recommendations of Bio-Rad (Hercules, Calif.).

Individual germ is isolated from mature kernels for determination of starch and oil concentrations of the seed part. Individual dry seed are soaked overnight at 4° C. in 1 mL of solution containing 20 mM acetate (pH 6.5) and 10 mM mercuric chloride (Adkins and Greenwood (1966) Starch 7: 213-218). Intact germ is dissected from the seed, dried by lyophilization and recorded for dry weight. Individual germ is ground for 10 seconds in a Silamet amalgam mixer and transferred with hexane washing into a microcentrifuge tube. The tissue is extracted by stirring with 1 mL of hexane 3×60 min and centrifuged after each extraction period. The supernatant of extractions is collected and placed into a preweighed aluminum pan. After evaporation of hexane from the weigh pans in a fumehood, final traces of solvent are removed in a forced draft oven at 105° C. for 15 minutes. Cooled weigh pans are reweighed to determine the total weight of oil extracted from the germ. The meal remaining after oil extraction is twice washed with water and centrifugation (10 min; 1,000×g) and analyzed for starch by a modified procedure for total starch measurement (McCleary et al. (1994) J Cereal Sci. 20: 51-58; 1994). Free sugars are removed by extraction with 80% ethanol and the starch dissolved in 90% dimethylsulfoxide. Heat stable α-amylase and high purity amyloglucosidase (very low in β-glucanse activities) are used to degrade the starch to monomeric carbohydrate. The resulting glucose is quantitated according to Jones et al. (1977) Plant Physiol. 60: 379-383; 1977 with modification to a microplate format.

### Example 3

### Cloning of LEC1. Construction of Expression Vectors and Transformation and Regeneration of Plants

Immature maize embryos from greenhouse or field grown High type II donor plants are bombarded with a plasmid containing a LEC1-encoding polynucleotide (FIG. 8). The LEC1-encoding polynucleotide is operably linked to the Ltp2 promoter and downstream to a PINII terminator, and a second LEC1-encoding sequence is operably linked to the G(amma)Z(ein)-W64A promoter and a downstream GZ-W64A terminator. The plasmid comprises the selectable marker gene PAT (Wohlleben et al. (1988) Gene 70: 25-37), which confers resistance to the herbicide Bialaphos. Alternatively, the selectable marker gene is provided on a separate plasmid. Transformation is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising a first LEC1-encoding polynucleotide operably linked to the Ltp2 promoter and a second LEC1-encoding polynucleotide operably linked to the ZM-W64A promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun #HE34-1 or #HE34-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for altered embryogenesis and oil content.

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 μl L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 gA MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15: 473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/i MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 4

### Construction of Expression Vectors Operably Linking an AGP Inhibitory Cassette with a LEC1 Expression Cassette and Transformation and Regeneration of Plants

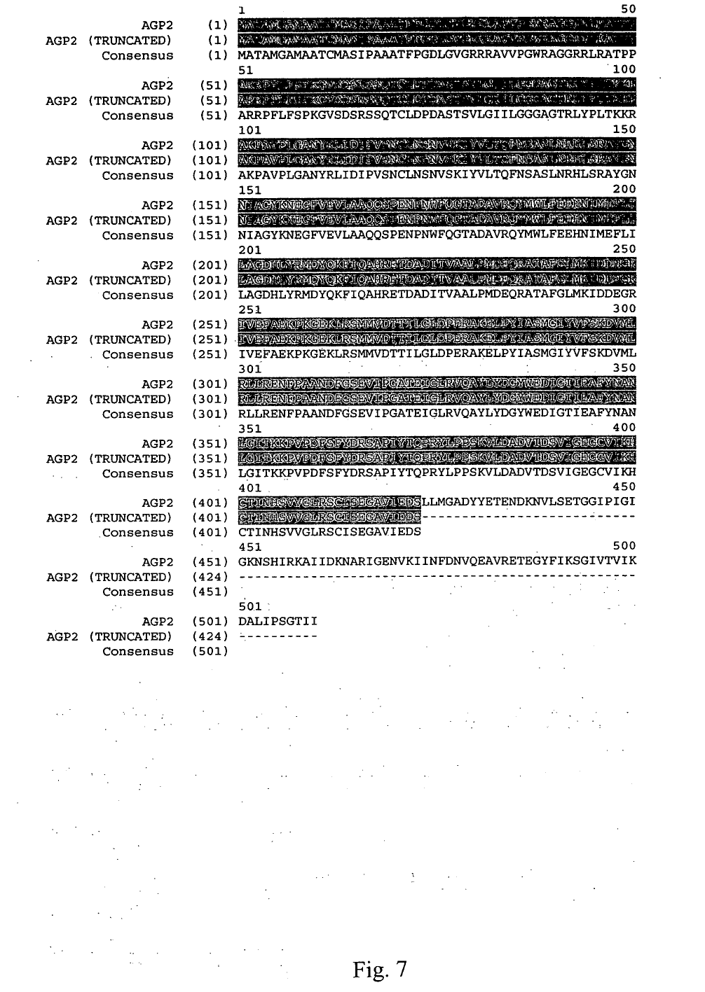

Expression cassettes comprising a polynucleotide encoding a starch biosynthetic inhibitor such as truncated AGP2 (truncated sequence shown in FIG. 7 and SEQ ID NO:6, encoded by SEQ ID NO:5) or truncated AGP2:AGP1 fusion (coding sequence of SEQ ID NO:15) and a growth stimulation polynucleotide, such as a LEC1-encoding polynucleotide (SEQ ID NO:1; see Example 3), having a selectable marker gene such as PAT (Wohlleben et al. (1988) Gene 70: 25-37), or bar gene (phosphinothricin acetyltransferase) for resistance to the herbicide phosphinothricin are constructed. The polynucleotides of interest are operably linked to independent transcriptional initiation regulatory sequences (see FIG. 6) that direct the transcription of the polynucleotides in the intended host cell, such as tissues of a transformed plant. The construction of such expression cassettes is well known to those of skill in the art in light of the present disclosure. See, for example, e.g., Sambrook et al. (1989) Molecular Cloning: A Laboratory Manual (Cold Spring Harbor, Laboratory Press, Plainview, N.Y.; Gelvin et al. Plant Molecular Biology Manual (1990); each incorporated herein in its entirety by reference.

Particle gun transformation is used; alternatively, transformation is carried out using an Agrobacterium co-transformation vector (see U.S. Pat. No. 5,981,840). Using the co-transformation system, the polynucleotide providing the growth advantage (i.e., comprising the sequence encoding LEC1) could be on a separate DNA construct from an agronomic gene linked to the AGP inhibitory sequence (i.e., sequence encoding truncated AGP2). Thus, the agronomic gene would be linked to the AGP and easily segregated away from the gene providing the growth advantage. In another embodiment, the AGP inhibitory sequence (i.e., encoding a truncated AGP2) could be placed in the same DNA construct as the polynucleotide providing the growth advantage (i.e., LEC1-encoding sequence) with the agronomic gene of interest on a separate DNA construct. In this case starch biosynthesis reduction could be used for primary transformant selection and embryogenesis for secondary transformant selection. Alternatively, the combined increase in oil content can be measured and compared to controls having a single cassette (see Example 1). A synergistic increase in oil content is indicative of dual expression.

Transformations can be performed as in Example 3.

### Example 5

### Construction of Expression Vectors for Decreasing Starch Biosynthesis Increasing Oil Production, and Modifying Characteristics of the Increased Oil, and Transformation and Regeneration of Plants

In order to inhibit starch biosynthesis, increase oil production, and modify the oil being produced, the following expression vectors were constructed:
(1) An expression vector (see plasmid C construct; FIG. 9) comprising:
   (a) a first expression cassette having a polynucleotide encoding the Zea mays LEC1 transcriptional activator (amino acid sequence set forth in SEQ ID NO:2, encoded by the sequence set forth in SEQ ID NO:1) operably linked to the LTP2A promoter, which expresses LEC1;
   (b) a second expression cassette having a polynucleotide comprising an AGP inhibitory sequence (see SEQ ID NO: 16) operably linked to the oleosin (OLE) promoter, which expresses an inhibitory nucleotide molecule that targets AGPase activity; the AGP inhibitory sequence comprises nucleotides corresponding to a region of AGP2 5′UTR, truncated AGP2 sequence (truncated region 1, designated AGP2 TR1), a polynucleotide spacer, sequence complementary to this truncated AGP2 sequence (designated AGP2 IR1), and sequence complementary to the region of AGP 5′UTR; and
   (c) a third expression cassette having a polynucleotide comprising an FAD2 inhibitory sequence (see SEQ ID NO:17) operably linked to the EAP1 promoter, which expresses an inhibitory nucleotide molecule that targets FAD2 activity; the FAD2 inhibitory sequence comprises nucleotides for a Zea mays truncated FAD2 sequence (truncated region 2, designated ZM-FAD2 TR2), another Zea mays truncated FAD2 sequence (truncated region 3, designated ZM-FAD2 TR3), a polynucleotide spacer, and sequence complementary to the ZM-FAD2 TR2 sequence; and
(2) an expression vector (see plasmid D construct; FIG. 10) comprising:
   (a) a first expression cassette having a polynucleotide encoding the Zea mays LEC1 transcriptional activator (amino acid sequence set forth in SEQ ID NO:2, encoded by the sequence set forth in SEQ ID NO:1) operably linked to the LTP2A promoter, which expresses LEC1; and
   (b) a second expression cassette having a polynucleotide comprising a dual AGP-FAD2 inhibitory sequence (see SEQ ID NO:18) operably linked to the OLE promoter, which expresses an inhibitory nucleotide molecule that targets both AGPase activity and desaturase activity of FAD2; the dual AGP-FAD2 inhibitory sequence comprises nucleotides corresponding to a region of AGP2 5′UTR, truncated AGP2 sequence (truncated region 3, designated AGP2 TR3), Zea mays truncated FAD2 sequence (truncated region 2, designated ZM-FAD2 TR2), another Zea mays truncated FAD2 sequence (truncated region 3A, designated ZM-FAD2 TR3A), an intronic nucleotide sequence (intron-1) from aldehyde dehydrogenase-1 (designated ADH1 INTRON1), another Zea mays truncated FAD2 sequence (truncated region 3B, designated ZM-FAD2 TR3B, sequence complementary to the ZM-FAD2 TR2 sequence, sequence complementary to the AGP2 TR3 sequence, and sequence complementary to the region of AGP 5′UTR.

See FIG. 11 for a linear map of AGP2 and FAD2 inhibitory sequence regions for plasmids C and D shown in FIGS. 9 and 10, respectively.

Transformed maize plants comprising these expression vectors were generated using methods of Example 7.

### Example 6

### Construction of Expression Vectors Operably Linking an FAD2 Inhibitory Cassette with a LEC1 Expression Cassette and Transformation and Regeneration of Plants

In order to sort out the contributory effects of inhibition of FAD2 and inhibition of AGP2 on oil production and oil traits, expression vectors comprising the following expression cassettes were constructed:
(1) an expression vector (plasmid E referred to in FIG. 18) comprising:
   (a) a first expression cassette having a polynucleotide encoding the Zea mays LEC1 transcriptional activator (amino acid sequence set forth in SEQ ID NO:2, encoded by the sequence set forth in SEQ ID NO:1) operably linked to the LTP2A promoter, which expresses LEC1; and
   (b) a second expression cassette having a polynucleotide comprising an FAD2 inhibitory sequence operably linked to the OLE promoter, which expresses an inhibitory nucleotide molecule (a cosuppression inhibitor of FAD2 in the sense orientation) that targets desaturase activity of FAD2; and
(2) an expression vector (plasmid F referred to in FIG. 18) comprising:
   (a) a first expression cassette having a polynucleotide encoding the Zea mays LEC1 transcriptional activator (amino acid sequence set forth in SEQ ID NO:2, encoded by the sequence set forth in SEQ ID NO:1) operably linked to the LTP2A promoter, which expresses LEC1; and
   (b) a second expression cassette having a polynucleotide comprising an FAD2 inhibitory sequence (see SEQ ID NO:19) operably linked to the OLE promoter, which expresses an inhibitory nucleotide molecule that targets FAD2 activity; the FAD2 inhibitory sequence comprises nucleotides for a Zea mays truncated FAD2 sequence (truncated region 2, designated ZM-FAD2 TR2), another Zea mays truncated FAD2 sequence (truncated region 3, designated ZM-FAD2 TR3), and sequence complementary to the ZM-FAD2 TR2 sequence.

Transformation and regeneration of plants and seed were performed according to the methods of Examples 7.

### Example 7

### Agrobacterium-Mediated Transformation

Immature maize embryos were transformed with a plasmid containing a polynucleotide of the invention (LEC1 of SEQ ID NO:1) (FIG. 8). The LEC1 polynucleotide was operably linked to the LTP2 promoter and downstream to a PINII terminator, and a second LEC1 gene was operably linked to the G(amma)Z(ein)-W64A promoter and a downstream GZ-W64A terminator (FIGS. 6 and 8). These sequences were duplicated as a single inverted region in the respective vectors.

For Agrobacterium-mediated transformation of maize with the LEC1 expression cassette and the AGP inhibitory cassette, the method of Zhao was employed (U.S. Pat. No. 5,981,840, and International Publication No. WO 98/32326; the contents of which are hereby incorporated by reference). Briefly, immature embryos were isolated from maize and the embryos contacted with a suspension of Agrobacterium, where the bacteria were capable of transferring the LEC1 expression cassette and an AGP inhibitory cassette to at least one cell of at least one of the immature embryos (step 1: the infection step). In this step the immature embryos were immersed in an Agrobacterium suspension for the initiation of inoculation. The embryos were co-cultured for a time with the Agrobacterium (step 2: the co-cultivation step). Generally, the immature embryos were cultured on solid medium following the infection step. Following this co-cultivation period an optional “resting” step was used. In this resting step, the embryos were incubated in the presence of at least one antibiotic known to inhibit the growth of Agrobacterium without the addition of a selective agent for plant transformants (step 3: resting step). Generally, the immature embryos were cultured on solid medium with antibiotic, but without a selecting agent, for elimination of Agrobacterium and for a resting phase for the infected cells. Next, inoculated embryos were cultured on medium containing a selective agent and growing transformed callus is recovered (step 4: the selection step). Generally, the immature embryos were cultured on solid medium with a selective agent resulting in the selective growth of transformed cells. The callus was then regenerated into plants (step 5: the regeneration step), and, generally, calli grown on selective medium were cultured on solid medium to regenerate the plants. Transformed plants were then grown and selected according to the methods in Example 3.

### Example 8

### Measurement of Oleic Acid Content of Transformed Seeds

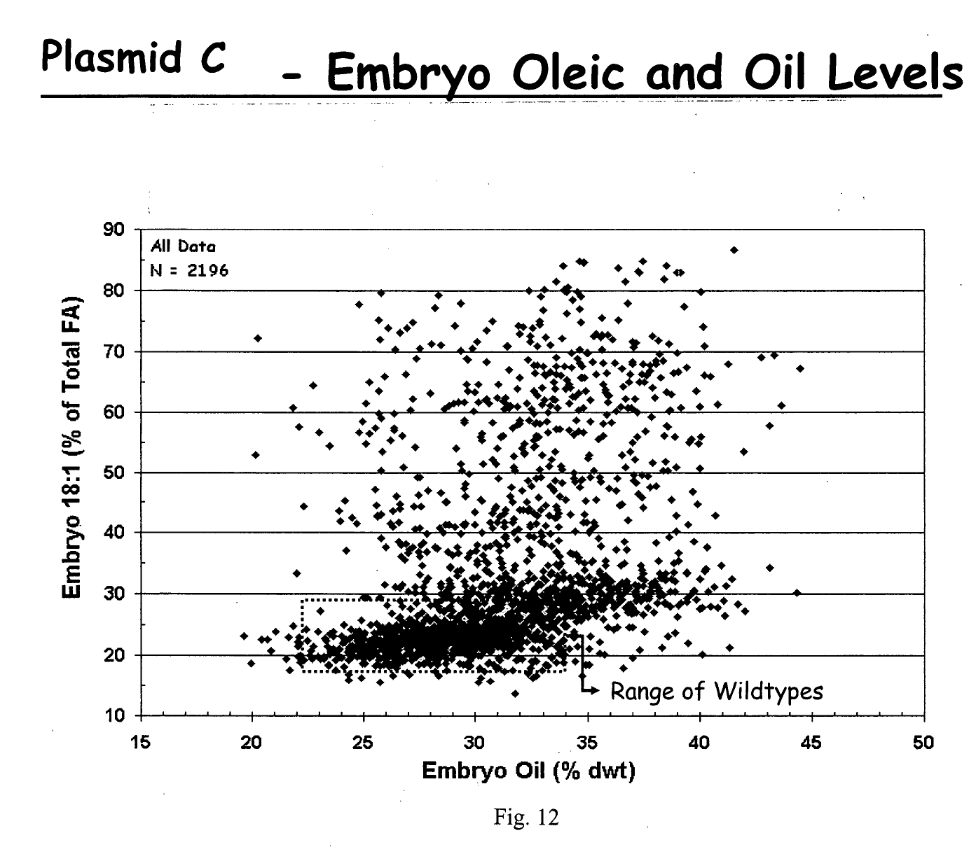

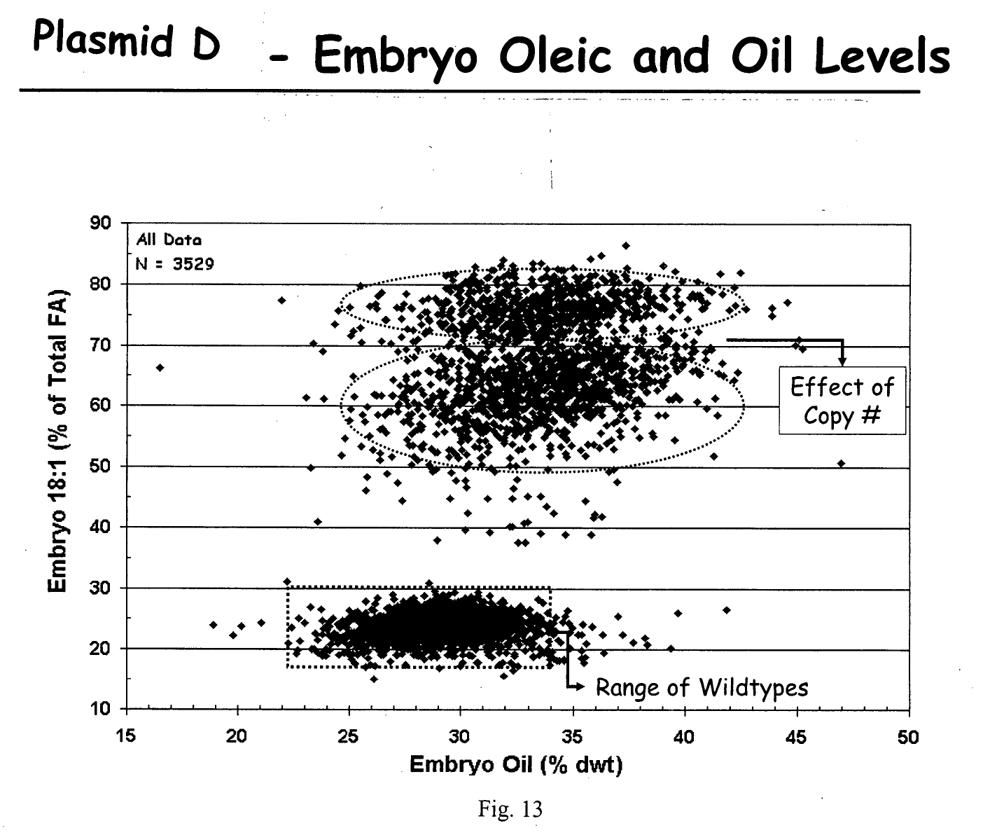

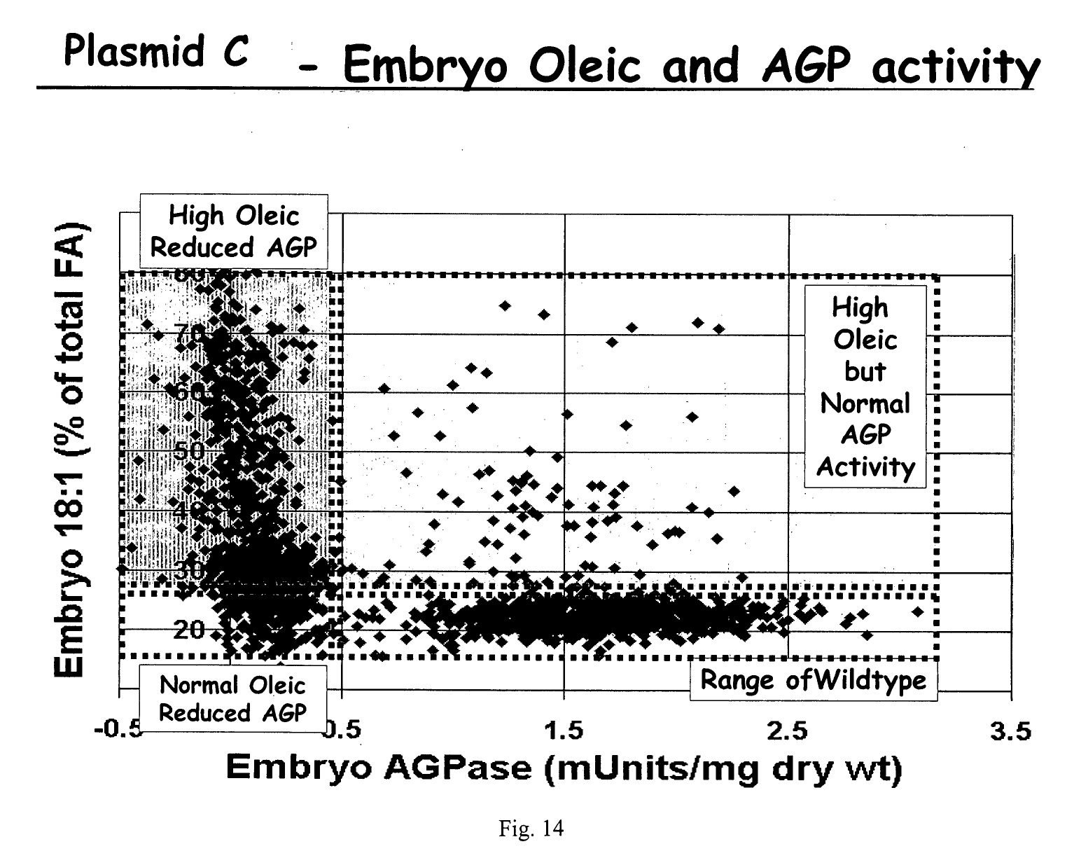

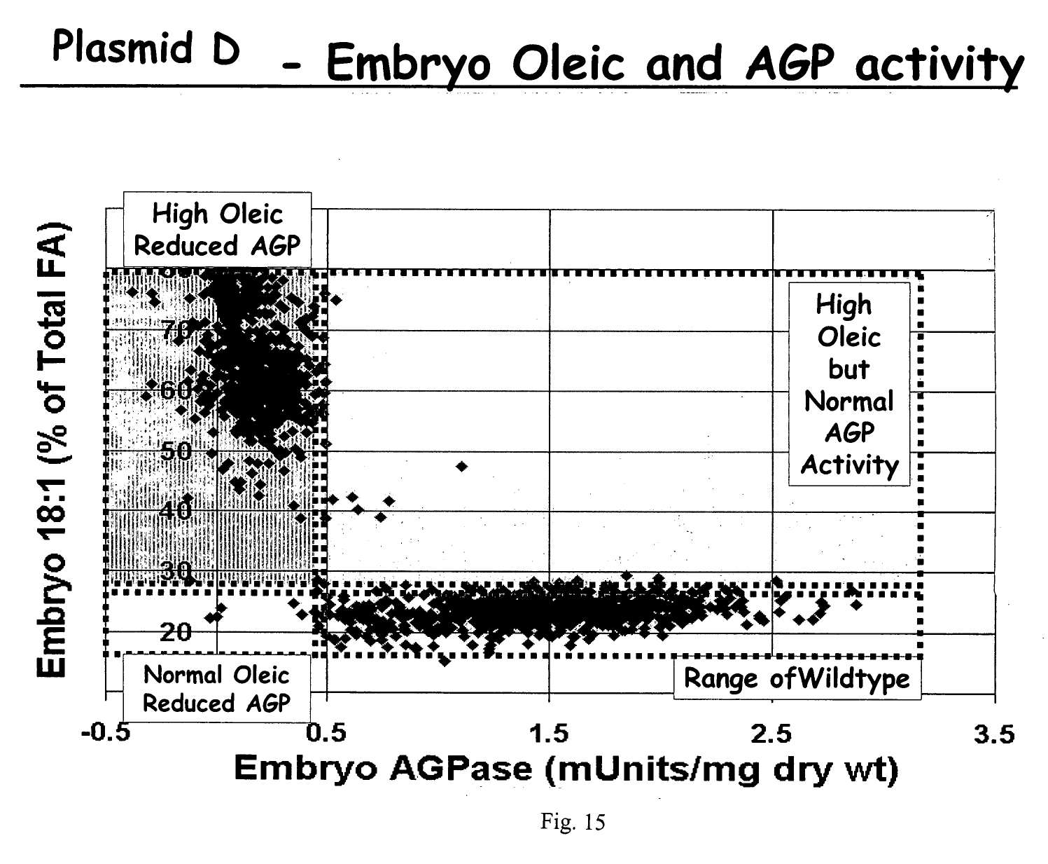

The oleic acid content of the seeds transformed by the constructs described in Examples 5 and 6 were measured using gas chromatography. In seed transformed with plasmid C(Ltp2::Lec|Ole::Agp2-IR|EAP::Fad2-IR), a continuum of oleic acid values was observed (FIG. 12). In seed transformed with plasmid D (Ltp2::Lec|Ole::(Agp2-Fad2-IR), the groups segregated into discrete populations with a subset segregating into expression level based on copy number (FIG. 13). When the expression of embryo oleic acid was compared to the cosuppression of AGP2 in plasmid C-transformed embryos, there was not strict cosegregation between transformation with the two genes and simultaneous alteration of AGPase activity and oleic acid content (FIG. 14). Conversely, when the expression of embryo oleic acid was compared to the cosuppression of AGP2 in plasmid D-transformed embryos, there was strict cosegregation between transformation with the two genes and simultaneous reduction of AGPase and increase of oleic acid content (FIG. 15). Changes in the oleic acid levels in transformed embryos may be dose dependent based on results for the expression level of the marker gene moPAT (maize optimized PAT under the control of the ubiquitin promoter) present in these vectors.

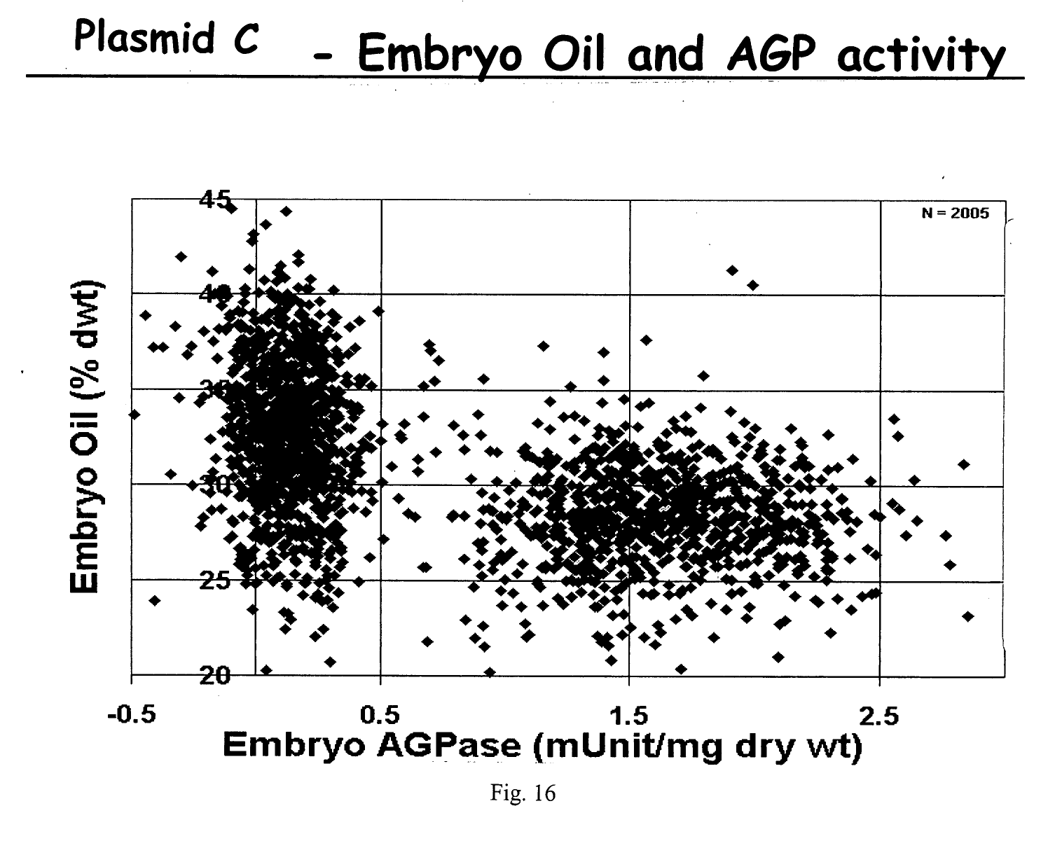

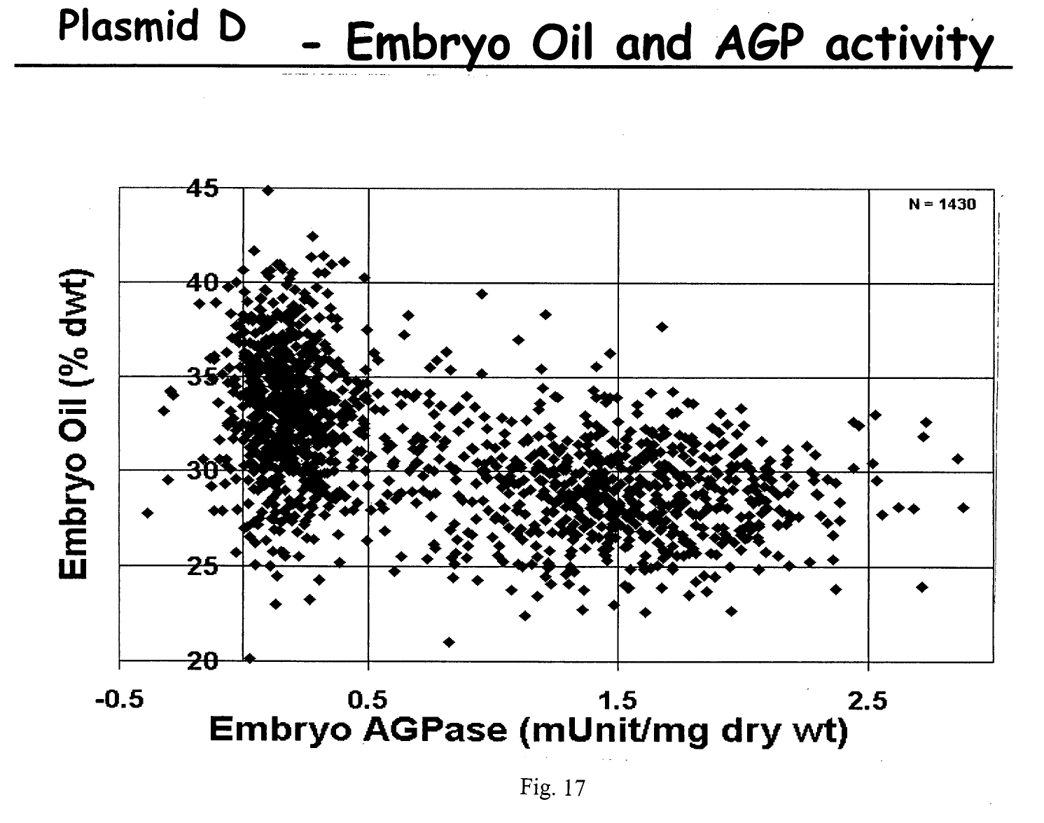

When the concentration of embryo total oil was compared to the cosuppression of AGP2 and FAD2 in plasmid C-transformed embryos, there was a distinct separation of AGPase activity, but with overlapping oil concentration (FIG. 16). When the concentration of embryo total oil was compared to the cosuppression of AGP2 and FAD2 in plasmid D-transformed embryos, there was a distinct separation of AGPase activity as well as more overlap of oil concentration (FIG. 17).

The total oil level in embryos transformed with the various plasmids discussed in Examples 5 and 6 were compared as shown in FIG. 18. Based on these results, plasmids C and D were selected for further experimentation because they produced the most oil.

### Example 9

### Soybean Embryo Transformation

Soybean embryos are bombarded with a plasmid containing the LEC1 expression cassette and an AGP inhibitory cassette (FIG. 6) and, optionally, an FAD2 inhibitory cassette (FIG. 9 or 10), as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327: 70-73, U.S. Pat. No. 4,945,050). A DuPont Biolistic PDS1000/HE instrument (helium retrofit) can be used for these transformations.

A selectable marker gene that can be used to facilitate soybean transformation is a transgene composed of the ³⁵S promoter from Cauliflower Mosaic Virus (Odell et al. (1985) Nature 313: 810-812), the hygromycin phosphotransferase gene from plasmid pJR225 (from E. coli; Gritz et al. (1983) Gene 25: 179-188), and the 3′ region of the nopaline synthase gene from the T-DNA of the Ti plasmid of Agrobacterium tumefaciens. The expression cassette comprising an LEC1 expression cassette and an AGP inhibitory cassette and, optionally, an FAD2 inhibitory cassette can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1M), and 50 μl CaCl₂ (2.5M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 10

### Sunflower Meristem Tissue Transformation

Sunflower meristem tissues are transformed with a vector containing a LEC1 expression cassette and an AGP inhibitory cassette (FIG. 6) and, optionally, an FAD2 inhibitory cassette (FIGS. 9 and 10), as follows (see also European Patent Number EP 0 486233, herein incorporated by reference, and Malone-Schoneberg et al. (1994) Plant Science 103: 199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer, et al. (Schrammeijer et al. (1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al. (1962) Physiol. Plant., 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney, et al. (1992) Plant Mol. Biol. 18: 301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA 05 is used in all transformation experiments. A binary plasmid vector comprising the expression cassette that contains the LEC1 expression cassette and an AGP inhibitory cassette and, optionally, an FAD2 inhibitory cassette is introduced into Agrobacterium strain EHA 05 via freeze-thawing as described by Holsters, et al. (1978) Mol. Gen. Genet. 163: 181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bactopeptone, and 5 gm/i NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for LEC1 activity, AGPase activity, FAD2 activity, and increased oil production, and/or forms of oil such as oleic acid and linoleic acid as discussed herein; see, for example, U.S. Pat. No. 6,232,529 B1 as discussed above, and U.S. Pat. No. 6,825,397; herein incorporated by reference in their entirety.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of To plants (parental generation) maturing in the greenhouse are identified by NPTII ELISA and expression of the selectable marker while transgenic seeds harvested from NPTII-positive To plants are identified by increased LEC1 activity, decreased AGP activity, and optionally decreased FAD2 activity in analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 μl MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour-day and 26° C. incubation conditions.

Explants (around 2 cm long) from two weeks of culture in 374C medium are screened for expression of the selectable marker using assays known in the art and disclosed herein. After positive (i.e., for increased LEC1 expression, decreased AGP expression, and optionally, decreased FAD2 expression) explants are identified, those shoots that fail to exhibit increased LEC1 and decreased AGP activity (and, optionally, decreased FAD2 activity) are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for increased LEC1 expression and decreased AGP activity (and, optionally, decreased FAD2 activity) are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

The article “a” and “an” are used herein to refer to one or more than one (i.e., to at least one) of the grammatical object of the article. By way of example, “an element” means one or more element.

Although the foregoing invention has been described in some detail by way of illustration and example for purposes of clarity of understanding, it will be obvious that certain changes and modifications may be practiced within the scope of the appended claims.

1. A method for increasing oil production in a plant, said method comprising increasing lipid biosynthesis in said plant in combination with disrupting starch biosynthesis, disrupting storage of starch, or disrupting both starch biosynthesis and storage of starch in said plant.

2. The method of claim 1, wherein starch biosynthesis is disrupted by a method selected from the group consisting of:
a) inhibiting expression or function of a gene product that is involved in starch biosynthesis;
b) increasing expression of a gene product that is involved in starch breakdown;
c) modifying activity of an enzyme that is involved in starch metabolism;
d) modifying a protein that is involved in nucleating starch biosynthesis;
e) modifying a protein that is involved in transport of assimilates that feed into the starch biosynthetic pathway;
f) eliminating a gene that is involved in starch metabolism;
g) mutation of a gene to effect reduced expression or activity of an enzyme that is involved in starch biosynthesis; and
h) shunting carbon away from a pathway for starch biosynthesis, thereby depriving the pathway of substrates for starch biosynthesis.

3. The method of claim 2, wherein said increasing of lipid biosynthesis comprises introducing into said plant a polynucleotide comprising a sequence encoding a transcriptional activator operably linked to a promoter that is functional in a plant cell, said transcriptional activator being capable of increasing lipid biosynthesis in said plant, wherein said transcriptional activator is selected from the group consisting of HAP3/LEC1 CAAT transcriptional activators, HAP2 transcriptional activators, HAP5 transcriptional activators, and MAPKK3/MEK transcriptional activators.

4. The method of claim 3, wherein said transcriptional activator comprises a conserved region corresponding to the consensus amino acid sequence set forth in SEQ ID NO:14.

5. The method of claim 3, wherein said method of disrupting starch biosynthesis comprises introducing into said plant a polynucleotide comprising a sequence that is capable of inhibiting expression or function of ADP-glucose pyrophosphorylase 1 (AGP1), AGP2, or a combination thereof.

6. The method of claim 5, wherein said transcriptional activator is a leafy cotyledon 1 transcriptional activator (LEC1).

7. The method of claim 3, further comprising disrupting lipid desaturase activity, wherein lipid desaturase activity is disrupted by a method selected from the group consisting of:
a) inhibiting expression or function of a gene product that is involved in desaturating fatty acids;
b) modifying activity of an enzyme that is involved in desaturating fatty acids;
c) eliminating a gene that is involved in saturated fatty acid metabolism; and
d) mutation of a gene to effect reduced expression or activity of an enzyme that is involved in desaturating fatty acids.

8. The method of claim 7, wherein said transcriptional activator is a leafy cotyledon 1 transcriptional activator (LEC1).

9. The method of claim 8, wherein said lipid desaturase activity is disrupted by introducing into said plant a polynucleotide comprising a sequence that is capable of inhibiting expression or function of fatty acid desaturase 2 (FAD2).

10. A nucleotide construct comprising a first polynucleotide having a sequence encoding a polypeptide having leafy cotyledon 1 transcriptional activator (LEC1) activity, wherein said polypeptide is selected from the group consisting of:
a) a polypeptide comprising an amino acid sequence encoded by the nucleotide sequence as set forth in SEQ ID NO:1;
b) a polypeptide comprising an amino acid sequence as set forth in SEQ ID NO:2;
c) a polypeptide comprising an amino acid sequence that is at least 60% identical to the amino acid sequence as set forth in SEQ ID NO:2;
d) a polypeptide comprising an amino acid sequence that is at least 70% identical to the amino acid sequence as set forth in SEQ ID NO:2;
e) a polypeptide comprising an amino acid sequence that is at least 80% identical to the amino acid sequence as set forth in SEQ ID NO:2;
f) a polypeptide comprising an amino acid sequence that is at least 90% identical to the amino acid sequence as set forth in SEQ ID NO:2;
g) a polypeptide comprising a conserved region corresponding to the consensus amino acid sequence set forth in SEQ ID NO:14; and
h) a functional fragment of the polypeptide according to any one of preceding items (a) through (g);
and a second polynucleotide comprising a sequence that is capable of inhibiting expression or function of ADP-glucose pyrophosphorylase 1 (AGP1), AGP2, or a combination thereof; wherein said sequence encoding said polypeptide having LEC1 activity and said sequence that is capable of inhibiting expression or function of AGP1, AGP2, or a combination thereof are operably linked to at least one promoter that is functional in a plant cell.

11. The nucleotide construct of claim 10, wherein a first promoter is operably linked to said sequence encoding said polypeptide having LEC1 activity and wherein a second promoter is operably linked to said sequence that is capable of inhibiting expression or function of AGP1, AGP2, or combination thereof, said first and second promoters being functional in a plant cell.

12. The nucleotide construct of claim 11, wherein said at least one promoter is a tissue-preferred promoter selected from the group consisting of seed-preferred, embryo-preferred, and endosperm-preferred promoters.

13. A vector comprising the nucleotide construct of claim 11.

14. A plant comprising the nucleotide construct of claim 11.

15. The plant of claim 14, wherein said plant is a monocot.

16. The plant of claim 14, wherein said plant is a dicot.

17. The plant of claim 14, wherein said plant is selected from the group consisting of corn, oat, soybean, wheat, rice, canola, Brassica sp., sorghum, sunflower, barley, millet, cotton, peanut, flax, safflower, palm, olive, castor bean, and coconut.

18. The nucleotide construct of claim 10, wherein said sequence encoding said polypeptide having LEC1 activity is selected from the group consisting of:
a) a nucleotide sequence as set forth in SEQ ID NO:1;
b) a nucleotide sequence that is at least 60% identical to the nucleotide sequence as set forth in SEQ ID NO:1;
c) a nucleotide sequence that is at least 70% identical to the nucleotide sequence as set forth in SEQ ID NO:1;
d) a nucleotide sequence that is at least 80% identical to the nucleotide sequence as set forth in SEQ ID NO:1;
e) a nucleotide sequence that is at least 90% identical to the nucleotide sequence as set forth in SEQ ID NO:1;
f) a nucleotide sequence encoding a polypeptide comprising a conserved region corresponding to the consensus amino acid sequence set forth in SEQ ID NO:14 or functional fragment thereof, and
g) a fragment of the nucleotide sequence according to any one of preceding items (a) through (f), wherein said fragment encodes a polypeptide having LEC1 activity.

19. The nucleotide construct of claim 10, wherein said sequence that is capable of inhibiting expression or function of AGP1, AGP2, or a combination thereof is selected from the group consisting of:
a) a nucleotide sequence as set forth in SEQ ID NO:3 or a complement thereof;
b) a nucleotide sequence as set forth in SEQ ID NO:5 or a complement thereof;
c) a nucleotide sequence as set forth in SEQ ID NO:15 or a complement thereof;
d) a nucleotide sequence that is at least 60% identical to the nucleotide sequence according to any one of preceding items (a) through (c);
e) a nucleotide sequence that is at least 70% identical to the nucleotide sequence according to any one of preceding items (a) through (c);
f) a nucleotide sequence that is at least 80% identical to the nucleotide sequence according to any one of preceding items (a) through (c);
g) a nucleotide sequence that is at least 90% identical to the nucleotide sequence according to any one of preceding items (a) through (c); and
h) a fragment of the nucleotide sequence according to any one of preceding items (a) through (g), wherein said fragment comprises at least 10 contiguous nucleotides of said nucleotide sequence.

20. The nucleotide construct of claim 19, wherein said sequence that is capable of inhibiting expression or function of AGP1, AGP2, or a combination thereof is a sequence that is capable of being transcribed as an inhibitory nucleotide molecule selected from the group consisting of a single-stranded RNA polynucleotide, a double-stranded RNA polynucleotide, and a combination thereof.

21. The nucleotide construct of claim 10, wherein said sequence that is capable of inhibiting expression or function of AGP1, AGP2, or a combination thereof comprises a nucleotide sequence selected from the group consisting of:
a) at least 10 contiguous nucleotides of the nucleotide sequence as set forth in SEQ ID NO:3 and at least 10 contiguous nucleotides of a complement thereof;
b) at least 10 contiguous nucleotides of the nucleotide sequence as set forth in SEQ ID NO:5 and at least 10 contiguous nucleotides of a complement thereof;
c) at least 10 contiguous nucleotides of the nucleotide sequence as set forth in SEQ ID NO:15 and at least 10 contiguous nucleotides of a complement thereof;
d) a nucleotide sequence that is at least 60% identical to the nucleotide sequence according to any one of preceding items (a) through (c);
e) a nucleotide sequence that is at least 70% identical to the nucleotide sequence according to any one of preceding items (a) through (c);
f) a nucleotide sequence that is at least 80% identical to the nucleotide sequence according to any one of preceding items (a) through (c); and
g) a nucleotide sequence that is at least 90% identical to the nucleotide sequence according to any one of preceding items (a) through (c).

22. The nucleotide construct of claim 21, wherein said sequence that is capable of inhibiting expression or function of AGP1, AGP2, or a combination thereof is a sequence that is capable of being transcribed as an inhibitory nucleotide molecule selected from the group consisting of a single-stranded RNA polynucleotide, a double-stranded RNA polynucleotide, and a combination thereof.

23. The nucleotide construct of claim 10, further comprising a third polynucleotide of interest.

24. The nucleotide construct of claim 23, wherein said third polynucleotide comprises a sequence that is capable of inhibiting expression or function of a fatty acid desaturase (FAD); wherein said sequence encoding said polypeptide having LEC1 activity, said sequence that is capable of inhibiting expression or function of AGP1, AGP2, or a combination thereof, and said sequence that is capable of inhibiting expression or function of said FAD are operably linked to at least one promoter that is functional in a plant cell.

25. The nucleotide construct of claim 24 wherein said sequence that is capable of inhibiting expression or function of AGP1, AGP2, or a combination thereof is a sequence that is capable of being transcribed as an inhibitory nucleotide molecule selected from the group consisting of a single-stranded RNA polynucleotide, a double-stranded RNA polynucleotide, and a combination thereof.

26. The nucleotide construct of claim 25, wherein said FAD is FAD2 and said construct comprises the sequence set forth in SEQ ID NO:18.

27. The nucleotide construct of claim 26, wherein said first promoter is an LTP2 promoter, and wherein said second promoter is an OLE promoter.

28. The nucleotide construct of claim 25, wherein said FAD is FAD2 and said sequence that is capable of inhibiting expression or function of said FAD2 comprises the sequence set forth in SEQ ID NO:17.

29. The nucleotide construct of claim 28, wherein said sequence that is capable of inhibiting expression or function of AGP1, AGP2, or a combination thereof comprises the sequence set forth in SEQ ID NO:16.

30. The nucleotide construct of claim 10, wherein said sequence that is capable of inhibiting expression or function of AGP1, AGP2, or a combination thereof is selected from the group consisting of:
a) a nucleotide sequence as set forth in SEQ ID NO:3 or a complement thereof,
b) a nucleotide sequence as set forth in SEQ ID NO:5 or a complement thereof,
c) a nucleotide sequence as set forth in SEQ ID NO:15 or a complement thereof,
d) a nucleotide sequence that is at least 60% identical to the nucleotide sequence according to any one of preceding items (a) through (c);
e) a nucleotide sequence that is at least 70% identical to the nucleotide sequence according to any one of preceding items (a) through (c);
f) a nucleotide sequence that is at least 80% identical to the nucleotide sequence according to any one of preceding items (a) through (c);
g) a nucleotide sequence that is at least 90% identical to the nucleotide sequence according to any one of preceding items (a) through (c); and
h) a fragment of the nucleotide sequence according to any one of preceding items (a) through (g), wherein said fragment comprises at least 10 contiguous nucleotides of said nucleotide sequence.

31. A nucleotide construct comprising a first nucleotide sequence capable of inhibiting expression or function of ADP-glucose pyrophosphorylase 1 (AGP1), AGP2, or a combination thereof and a second nucleotide sequence capable of inhibiting expression or function of a fatty acid desaturase (FAD); further wherein said first nucleotide sequence and said second nucleotide sequence are operably linked to at least one promoter that is functional in a plant cell.

32. The construct of claim 31, wherein said sequence capable of inhibiting expression or function of ADP-glucose pyrophosphorylase 1 (AGP1), AGP2, or a combination thereof comprises the sequence as set forth in SEQ ID NO:16.

33. The construct of claim 31, wherein said nucleotide sequence capable of inhibiting expression or function of a fatty acid desaturase (FAD) comprises the sequence as set forth in SEQ ID NO:17.

34. The construct of claim 31, wherein said first nucleotide sequence and said second nucleotide sequence are operably linked to each other as a fusion polynucleotide, further wherein said fusion polynucleotide is operably linked to a single promoter that is functional in a plant cell.

35. The construct of claim 34, wherein said fusion polynucleotide is expressed as a hairpin RNA.

36. The construct of claim 35, wherein said hairpin RNA comprises SEQ ID NO:18.

37. A plant comprising the construct of claim 32.

38. A plant comprising the construct of claim 33.

39. A plant comprising the construct of claim 36.

40. A method for altering oil phenotype of a plant, said method comprising introducing into said plant a nucleotide construct comprising a first nucleotide sequence capable of inhibiting expression or function of ADP-glucose pyrophosphorylase 1 (AGP1), AGP2, or a combination thereof and a second nucleotide sequence capable of inhibiting expression or function of a fatty acid desaturase (FAD); further wherein said first nucleotide sequence and said second nucleotide sequence are operably linked to at least one promoter that is functional in a plant cell.

41. A nucleotide construct comprising a polynucleotide selected from the group consisting of SEQ ID NO:16, SEQ ID 17, and SEQ ID NO:18.
